(19) United States
(12) Patent Application Publication
(10) Pub. No.: US 2023/0041738 A1
(43) Pub. Date: Feb. 9, 2023

(54) 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Yam B. POUDEL, Fremont, CA (US); Matthew COX, San Francisco, CA (US); Liqi HE, San Jose, CA (US); Ashvinikumar V. GAVAI, Princeton Junction, NJ (US); Sanjeev GANGWAR, Foster City, CA (US); Mathias BROEKEMA, New Hope, PA (US); Christine M. TARBY, Lawrenceville, NJ (US); Murugaiah ANDAPPAN MURUGAIAH SUBBAIAH, Bangalore (IN)

(73) Assignee: BRISTOL-MYERS SQUIBB COMPANY, Princeton, NJ (US)

(21) Appl. No.: 17/793,174

(22) PCT Filed: Jan. 26, 2021
(86) PCT No.: PCT/US2021/014982

§ 371 Date: Jul. 15, 2022

Related U.S. Application Data
(60) Provisional application No. 62/966,119, filed on Jan. 27, 2020.

(51) Int. Cl. A61K 31/519; C07D 519/00; C07D 487/04; A61K 31/5386; A61K 39/395

(52) U.S. Cl. CPC A61K 31/519; C07D 519/00; C07D 487/04; A61K 31/5386; A61K 39/3955

(57) ABSTRACT

Compounds according to formula I are useful as agonists of Toll-like receptor 7 (TLR7). (I) Such compounds can be used in cancer treatment, especially in combination with an anti-cancer immunotherapy agent, or as a vaccine adjuvant.

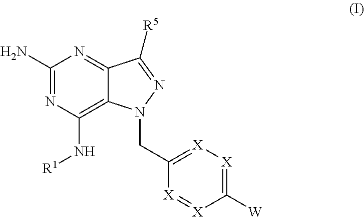

### CROSS-REFERENCE TO RELATED APPLICATIONS

This application claims the benefit under 35 U.S.C. § 119(e) of U.S. Provisional Application Ser. No. 62/966,119, filed Jan. 27, 2020, the disclosure of which is incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghöfer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

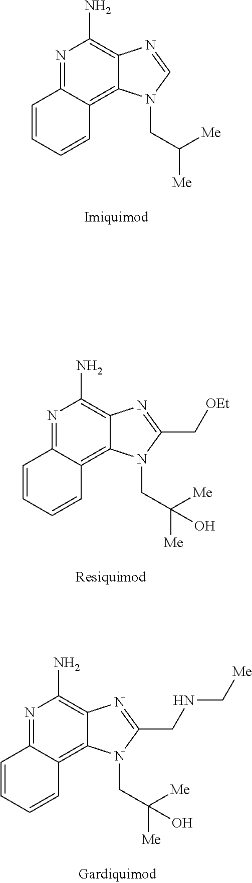 [SMILES: CC(C)Cn1cnc2c(N)nc3ccccc3c21 ; CCOCc1nc2c(N)nc3ccccc3c2n1CC(C)(C)O ; CCNCc1nc2c(N)nc3ccccc3c2n1CC(C)(C)O]

Synthetic TLR7 agonists based on a pteridinone molecular scaffold are also known, as exemplified by vesatolimod (Desai et al. 2015).

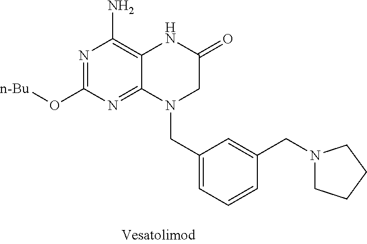

Other synthetic TLR7 agonists based on a purine-like scaffold have been disclosed, frequently according to the general formula (A):

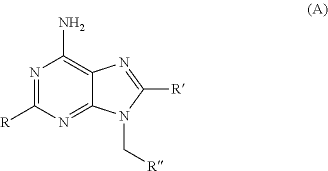

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system, having activity as TLR7 agonists.

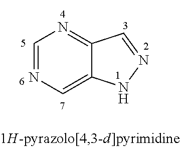

In one aspect, there is provided a compound with a structure according to formula I

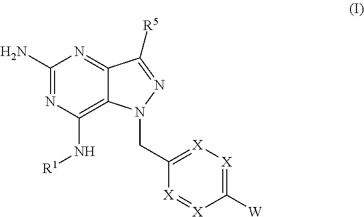

wherein

### W is

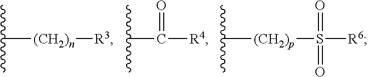

each X is independently N or CR²;
R¹ is (C₁-C₅ alkyl),
(C₂-C₅ alkenyl),
(C₁-C₈ alkanediyl)₀₋₁(C₃-C₆ cycloalkyl),
(C₂-C₈ alkanediyl)OH,
(C₂-C₈ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
(C₂-C₈ alkanediyl)₀₋₁(C₃-C₆ cycloalkanediyl)(C₃-C₆ cycloalkyl),
or
(C₂-C₈ alkanediyl)NR^{x}R^{y};
   each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
   R³ is NH[C(═O)]₀₋₁(C₁-C₄ alkanediyl)₀₋₁(C₄-C₁₀ bicycloalkyl),
O(C₁-C₄ alkanediyl)₀₋₁(C₄-C₈ bicycloalkyl),
or
a moiety having the structure

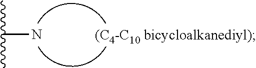 [SMILES: *N]

R⁴ is NH(C₁-C₄ alkanediyl)₀₋₁(C₄-C₁₀ bicycloalkyl)
or
a moiety having the structure

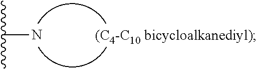 [SMILES: *N]

R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl,

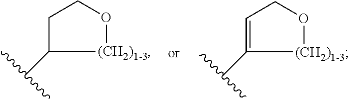

R⁶ is (NH)₀₋₁(C₁-C₄ alkanediyl)₀₋₁(C₄-C₁₀ bicycloalkyl),
or
a moiety having the structure

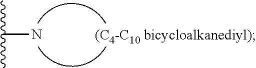 [SMILES: *N]

R^{x} and R^{y} are independently H or C₁-C₃ alkyl or R^{x} and R^{y} combine with the nitrogen to which they are bonded to form a 3- to 7-membered heterocycle;
n is 1, 2, or 3;
and
p is 0, 1, 2, or 3;
wherein in R¹, R², R³, R⁴, R⁵, and R⁶
an alkyl, alkenyl, cycloalkyl, alkanediyl, bicycloalkyl, or a moiety of the formula

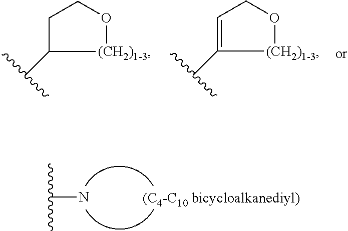

is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkenyl, alkanediyl, cycloalkyl, bicycloalkyl, or a moiety of the formula

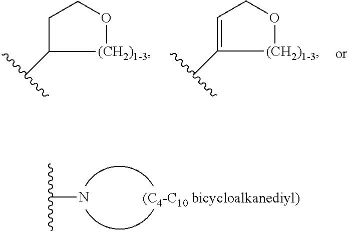

optionally may have a CH₂ group replaced by O, SO₂, CF₂, C(═O), NH,
N[C(═O)]₀₋₁(C₁-C₅ alkyl),
N[C(═O)]₀₋₁(C₁-C₄ alkanediyl)CF₃,
N[C(═O)]₀₋₁(C₂-C₄ alkanediyl)OH
N(SO₂)(C₁-C₃ alkyl),
N(C₁-C₃ alkanediyl)₀₋₁[C(═O)]N(C₁-C₃ alkyl)₂,
or
N[C(═O)]₀₋₁(C₁-C₄ alkanediyl)₀₋₁(C₃-C₅ cycloalkyl);
   with the proviso that the compound of formula (I) is not

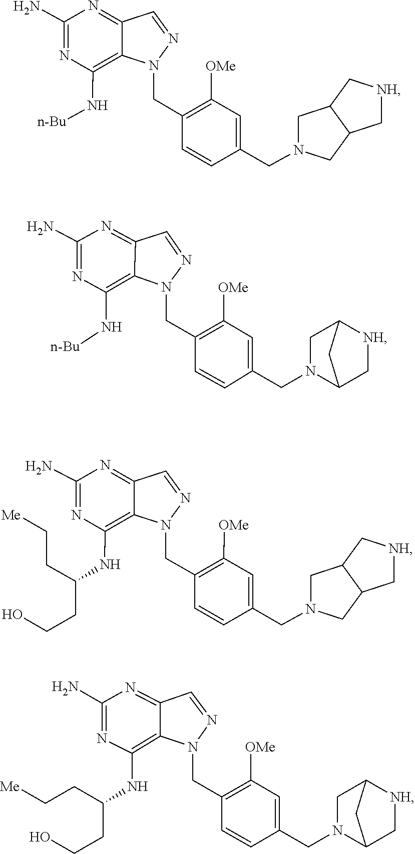

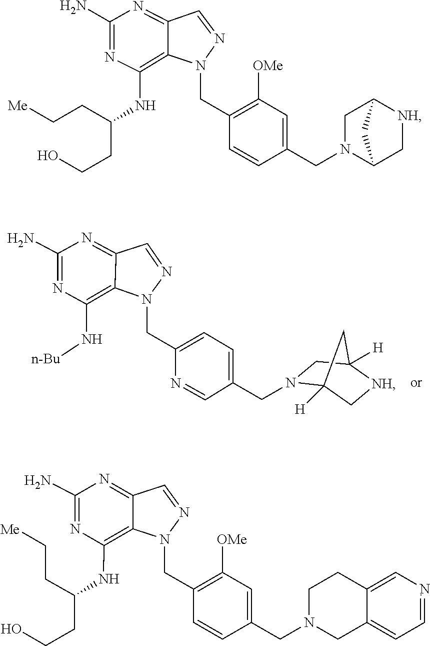

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In one aspect, compounds of this disclosure are according to formula (Ia), wherein R¹, R², R⁵, and W are as defined in respect of formula (I):

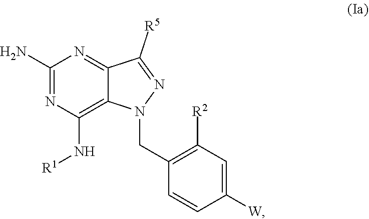

where R² preferably is OMe.

In another aspect, this disclosure provides a compound having a structure according to formula (Ia)

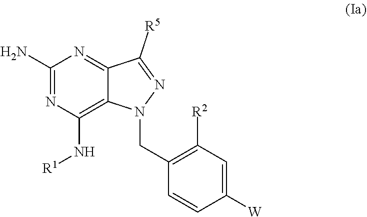

wherein

### R¹ is

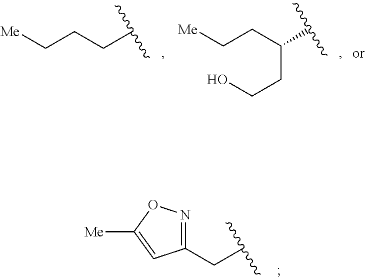 [SMILES: *CCCC ; *[C@@H](CCC)CCO ; *Cc1cc(C)on1]

R² is OMe or OCHF₂;

### R⁵ is H or Me; and

### W is

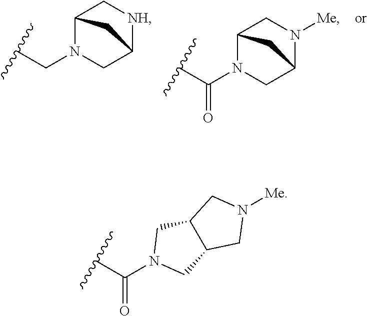 [SMILES: *CN1C[C@@H]2C[C@H]1CN2 ; *C(=O)N1C[C@@H]2C[C@H]1CN2C ; *C(=O)N1C[C@H]2CN(C)C[C@H]2C1]

In another aspect, compounds of this disclosure are according to formula (Ib), wherein R¹, R², R³, and R⁵ are as defined in respect of formula (I):

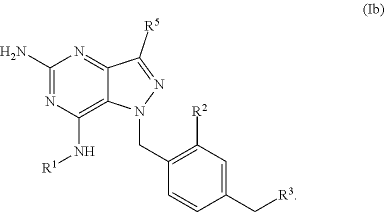

In another aspect, compounds of this disclosure are according to formula (Ic), wherein R¹, R², R⁴, and R⁵ are as defined in respect of formula (I):

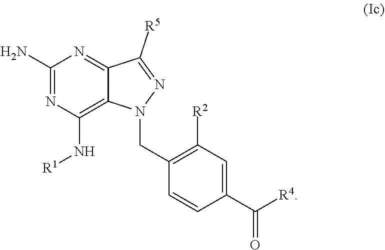

In another aspect, this disclosure provides a compound according to formula (Id), wherein R¹, R², R³, and R⁵ are as defined in respect of formula (I) and one X is N and the other X is CH:

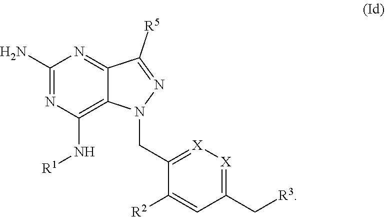

In formula (Id), preferably R¹ is

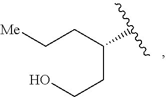 [SMILES: *[C@@H](CCC)CCO]

R² is OMe, and R⁵ is H.

Examples of suitable groups R¹ include:

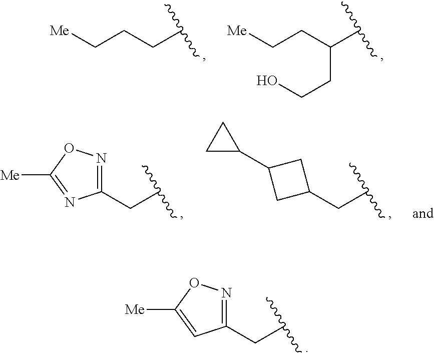 [SMILES: *CCCC ; *C(CCC)CCO ; *Cc1noc(C)n1 ; *CC1CC(C2CC2)C1 ; *Cc1cc(C)on1]

In one aspect, R¹ is selected from the group consisting of

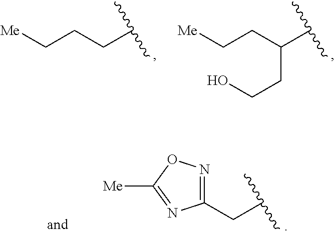 [SMILES: *CCCC ; *C(CCC)CCO ; *Cc1noc(C)n1]

R² preferably is OMe, O(cyclopropyl), or OCHF₂, more preferably OMe or OCHF₂, and especially preferably OMe.

R⁵ preferably is H, CH₂OH, or Me, more preferably H.

Examples where W is

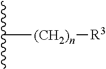

with n equals 1 include:

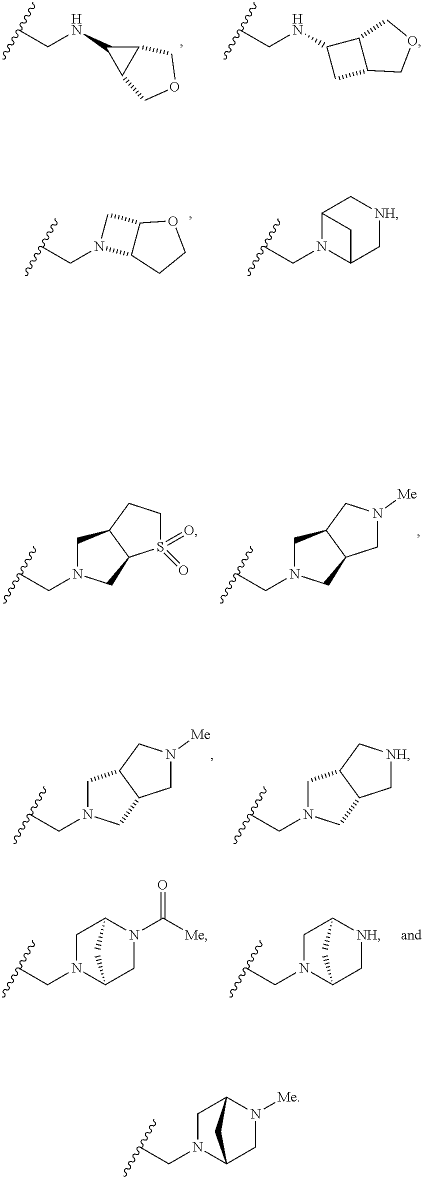

In one aspect,

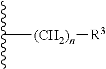

is

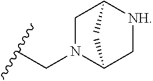 [SMILES: *CN1C[C@@H]2C[C@H]1CN2]

Examples where W is

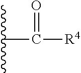

include:

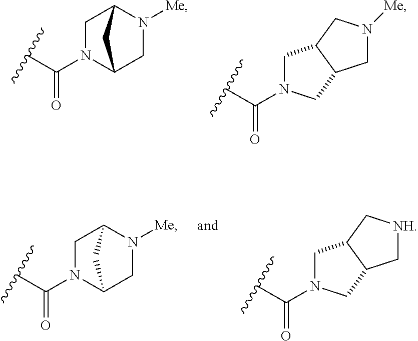

In one aspect, W is

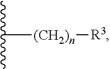

preferably with n equals 1.

In one aspect, W is

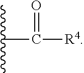

By way of exemplification and not of limitation, bicycloalkyl groups include

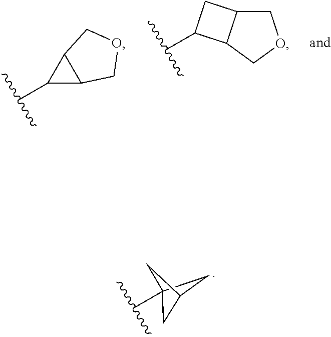 [SMILES: *C1C2COCC12 ; *C1CC2COCC12 ; *C12CC(C1)C2]

By way of exemplification and not of limitation, moieties of the formula

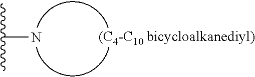 [SMILES: *N]

include

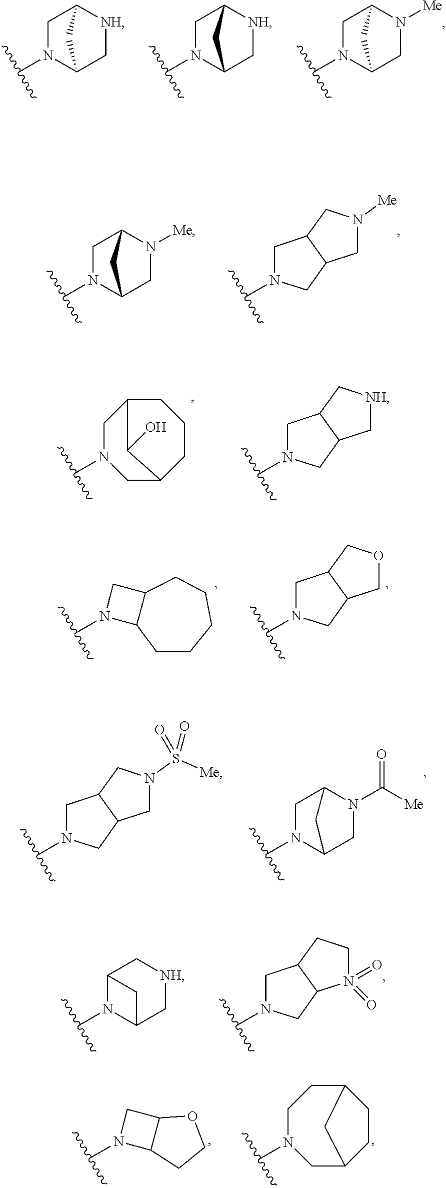

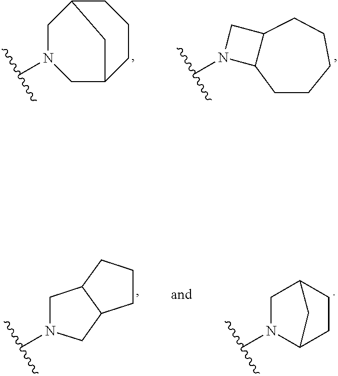

Some of the above exemplary bicycloalkyl groups and moieties of the formula

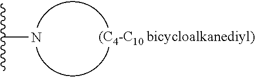 [SMILES: *N]

bear optional substituents and/or optionally have one or more CH₂ groups replaced by 0, SO₂, etc., as described in the BRIEF SUMMARY OF THE DISCLOSURE above.

Specific examples of compounds disclosed herein are shown in the following Table A. The table also provides data relating to biological activity: human TLR7 reporter assay and/or induction of the CD69 gene in human whole blood, determined per the procedure provided hereinbelow. The right-most column contains analytical data (mass spectrum, HPLC retention time, and NMR). In one embodiment, a compound of this disclosure has (a) a human TLR7 (hTLR7) Reporter Assay EC₅₀ value of less than 1,000 nM and (b) a human whole blood (hWB) CD69 induction EC₅₀ value of less than 1,000 nM. (Where an assay was performed multiple times, the reported value is an average.)

TABLE A

| | | hTLR7 | hWB | Analytical Data (Mass spectrum, LC/MS |
|---|---|---|---|---|
| Cpd | | Reporter | CD69 | Retention Time, ¹H NMR (500 MHz, |
| No. | Structure and Name | EC50, nM | EC50, nM | DMSO-d6 unless noted otherwise)) |
| 101 | 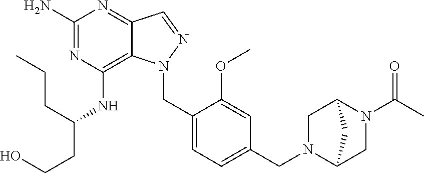 [SMILES: CCC[C@@H](CCO)Nc1nc(N)nc2cnn(Cc3ccc(CN4C[C@@H]5C[C@H]4CN5C(C)=O)cc3OC)c12] 1-[(1S,4S)-5-({4-[(5-amino-7-{[(3S)- 1-hydroxyhexan-3-yl]amino}-1H- pyrazolo[4,3-d]pyrimidin-1- yl)methyl]-3-methoxypheyl}- methyl)-2,5-diazabicyclo[2.2.1]- heptan-2-yl]ethan-1-one | 28.8 | | LC/MS [M + H]⁺ = 523.1 RT (min) = 1.03 (Procedure B) δ 7.57 (s, 1H), 7.04 (s, 1H), 6.81 (t, J = 6.9 Hz, 1H), 6.40 (dd, J = 8.1, 2.4 Hz, 1H), 5.70- 5.62 (m, 3H), 5.56 (d, J = 16.0 Hz, 1H), 4.50 (s, 1H), 4.33 (s, 1H), 3.86 (s, 2H), 3.66 (d, J = 12.8 Hz, 1H), 3.29 (d, J = 9.0 Hz, 1H), 3.07 (d, J = 10.7 Hz, 1H), 2.80 (s, 0H), 2.75 (s, 0H), 1.93 (d, J = 16.9 Hz, 6H), 1.87 (s, 2H), 1.66 (dd, J = 18.9, 9.4 Hz, 2H), 1.58 (d, J = 9.6 Hz, 1H), 1.51 (s, 1H), 1.44-1.32 (m, 2H), 1.06 (d, J = 7.9 Hz, 2H), 0.77 (t, J = 7.3 Hz, 3H) |
| 102 | 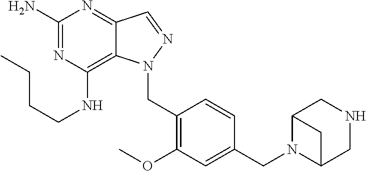 [SMILES: CCCCNc1nc(N)nc2cnn(Cc3ccc(CN4C5CNCC4C5)cc3OC)c12] N7-butyl-1-{[4-({3,6-diazabicyclo- [3.1.1]heptan-6-yl}methyl)-2- methoxyphenyl]methyl}-1H-pyrazolo- [4,3-d]pyrimidine-5,7-diamine | 89.6 | | LC/MS [M + H]⁺ = 437.2 RT (min) = 1.02 (Procedure B) δ 8.38 (s, 0H), 8.33 (s, 1H), 7.94 (d, J = 14.6 Hz, 1H), 7.76 (d, J = 6.0 Hz, 1H), 7.24 (s, 0H), 7.18 (d, J = 12.1 Hz, 1H), 7.04 (dd, J = 25.1, 17.3 Hz, 1H), 6.93 (d, J = 7.7 Hz, 0H), 6.86 (d, J = 7.6 Hz, 1H), 5.74 (s, 2H), 4.23 (s, 1H), 3.79 (s, 2H), 2.90 (s, 1H), 2.74 (s, 1H), 1.62-1.55 (m, 2H), 1.31-1.24 (m, 2H), 0.89 (t, J = 7.3 Hz, 3H) |
| 103 | 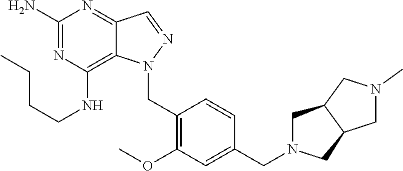 [SMILES: CCCCNc1nc(N)nc2cnn(Cc3ccc(CN4C[C@H]5CN(C)C[C@H]5C4)cc3OC)c12] 1-[(4-{[(3aR,6aS)-5-methyl-octa- hydropyrrolo[3,4-c]pyrrol-2-yl] methyl}-2-methoxyphenyl)methyl]- N7-butyl-1H-pyrazolo[4,3- d]pyrimidine-5,7-diamine | 27.8 | | LC/MS [M + H]⁺ = 464.9 RT (min) = 1.03 (Procedure B) δ 7.56 (d, J = 1.4 Hz, 1H), 6.98 (s, 1H), 6.76 (d, J = 7.7 Hz, 1H), 6.40 (d, J = 7.7 Hz, 2H), 5.67 (s, 1H), 5.61 (s, 2H), 3.83 (s, 2H), 3.41- 3.35 (m, 1H), 2.55 (s, 4H), 2.29 (d, J = 3.2 Hz, 2H), 2.24 (d, J = 10.4 Hz, 5H), 1.91 (d, J = 1.6 Hz, 3H), 1.45 (p, J = 7.2 Hz, 2H), 1.17 (q, J = 7.6 Hz, 2H), 0.83 (t, J = 7.4 Hz, 3H) |
| 104 | 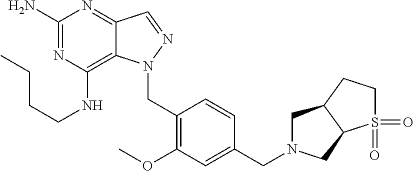 [SMILES: CCCCNc1nc(N)nc2cnn(Cc3ccc(CN4C[C@H]5CCS(=O)(=O)[C@H]5C4)cc3OC)c12] (3aR,6aR)-5-[(4-{[5-amino-7-(butyl- amino)-1H-pyrazolo[4,3-d]pyrimidin- 1-yl]methyl}-3-methoxyphenyl) methyl]-hexahydro-2H-1λ6- thieno[2,3-c] pyrrole-1,1-dione | 196.7 | | LC/MS [M + H]⁺ = 500.1 RT (min) = 0.84 (Procedure B) δ 8.30 (s, 1H), 7.91 (s, 1H), 7.77 (s, 1H), 7.28 (s, 1H), 7.17 (s, 1H), 7.11 (s, 1H), 7.07 (s, 1H), 6.90 (s, 1H), 6.80 (d, J = 7.6 Hz, 1H), 5.73 (s, 2H), 3.78 (s, 3H), 3.59 (dd, J = 17.1, 10.0 Hz, 2H), 3.46 (s, 1H), 3.15 (dd, J = 12.7, 8.5 Hz, 1H), 2.19-2.11 (m, 1H), 2.00 (d, J = 13.5 Hz, 1H), 1.57 (q, J = 7.3 Hz, 2H), 1.26 (q, J = 7.2 Hz, 5H), 0.88 (t, J = 7.3 Hz, 3H) |
| 105 | 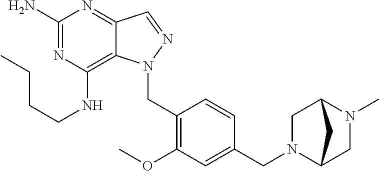 [SMILES: CCCCNc1nc(N)nc2cnn(Cc3ccc(CN4C[C@H]5C[C@@H]4CN5C)cc3OC)c12] N7-butyl-1-[(2-methoxy-4- {[(1R,4R)-5-methyl-2,5-diaza- bicyclo[2.2.1]heptan-2-yl]methyl}- phenyl)methyl]-1H-pyrazolo[4,3- d]pyrimidine-5,7-diamine | 20.7 | 4.2 | LC/MS [M + H]⁺ = 451.2 RT (min) = 0.873 (Procedure B) δ 7.55 (d, J = 1.2 Hz, 1H), 7.01 (s, 1H), 6.78 (d, J = 7.8 Hz, 1H), 6.42 (d, J = 7.5 Hz, 2H), 5.66 (s, 2H), 5.60 (s, 2H), 3.83 (d, J = 1.8 Hz, 3H), 3.68-3.57 (m, 1H), 3.39 (d, J = 6.3 Hz, 1H), 3.18 (s, 0H), 2.82 (d, J = 9.7 Hz, 1H), 1.91 (d, J = 2.0 Hz, 6H), 1.66 (s, 2H), 1.46 (p, J = 7.0 Hz, 3H), 1.18 (p, J = 7.1 Hz, 3H), 0.83 (t, J = 7.3 Hz, 4H) |
| 106 | 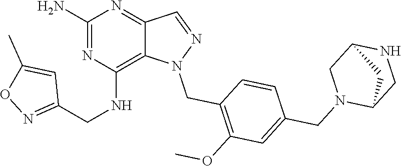 [SMILES: COc1cc(CN2C[C@@H]3C[C@H]2CN3)ccc1Cn1ncc2nc(N)nc(NCc3cc(C)on3)c21] 1-[(4-{[(1S,4S)-2,5-diazabicyclo- [2.2.1]heptan-2-yl]methyl}-2- methoxyphenyl)methyl]-N7-[(5- methyl-1,2-oxazol-3-yl)methyl]-1H- pyrazolo[4,3-d]pyrimidine-5,7- diamine | 234.0 | 73.6 | LC/MS [M + H]⁺ = 476.1 RT (min) = 0.63 (Procedure 3) ¹H NMR (400 MHz, DMSO-d₆) δ 7.57 (s, 1H), 7.24 (br t, J = 5.7 Hz, 1H), 6.95 (s, 1H), 6.76 (d, J = 7.2 Hz, 1H), 6.46 (d, J = 7.7 Hz, 1H), 6.00 (d, J = 0.8 Hz, 1H), 5.74 (s, 2H), 5.59 (s, 2H), 4.63 (d, J = 5.5 Hz, 2H), 3.75 (s, 3H), 3.66-3.56 (m, 2H), 3.01 (br d, J = 9.8 Hz, 1H), 2.72-2.64 (m, 2H), 2.33 (d, J = 0.7 Hz, 3H), 2.31 (br s, 1H), 1.68 (br d, J = 9.2 Hz, 1H), 1.41 (br d, J = 9.2 Hz, 1H) |
| 107 | 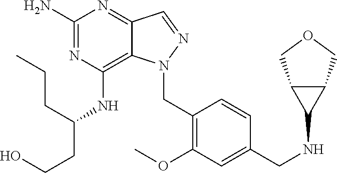 (3S)-3-[(5-amino-1-{[2-methoxy-4- ({[(1R,5S,6S)-3-oxabicyclo[3.1.0]- hexan-6-yl]amino}methyl)phenyl]- methyl}-1H-pyrazolo[4,3-d] pyrimidin-7-yl)amino]hexan-1-ol | 5.3 | | LC/MS [M + H]⁺ = 481.9 RT (min) = 1.05 (Procedure B) δ 7.58 (s, 1H), 7.04 (s, 1H), 6.78 (d, J = 7.8 Hz, 1H), 6.42 (d, J = 7.6 Hz, 1H), 5.76- 5.63 (m, 3H), 5.56 (d, J = 16.9 Hz, 1H), 4.34 (s, 1H), 3.86 (s, 3H), 3.54 (d, J = 8.0 Hz, 0H), 3.33 (t, J = 6.6 Hz, 1H), 2.56 (s, 4H), 1.93 (s, 2H), 1.81 (d, J = 2.5 Hz, 1H), 1.65 (dd, J = 13.2, 6.0 Hz, 1H), 1.61 (s, 2H), 1.56- 1.50 (m, 1H), 1.46-1.33 (m, 2H), 1.09 (s, 1H), 0.78 (t, J = 7.3 Hz, 3H) |
| 108 | 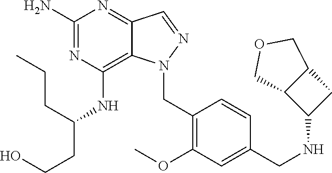 (3S)-3-[(5-amino-1-{[2-methoxy-4- ({[(1R,5R,6S)-3-oxabicyclo- [3.2.0] heptan-6-yl]amino} methyl)phenyl]methyl}-1H-pyrazolo- [4,3-d]pyrimidin-7-yl)amino]hexan- 1-ol | 4.9 | | LC/MS [M + H]⁺ = 496.3 RT (min) = 0.84 (Procedure B) δ 7.57 (s, 1H), 7.04 (s, 1H), 6.77 (d, J = 7.8 Hz, 1H), 6.39 (d, J = 7.7 Hz, 1H), 5.67 (d, J = 15.5 Hz, 3H), 5.55 (d, J = 16.9 Hz, 1H), 4.33 (s, 1H), 4.05 (d, J = 9.6 Hz, 1H), 3.86 (s, 3H), 3.58-3.49 (m, 1H), 3.33 (d, J = 6.9 Hz, 1H), 3.24 (dd, J = 8.8, 4.4 Hz, 1H), 3.16 (q, J = 8.0 Hz, 1H), 2.93-2.87 (m, 1H), 2.63 (d, J = 12.5 Hz, 1H), 2.28 (q, J = 9.9, 9.2 Hz, 1H), 1.92 (s, 3H), 1.68-1.61 (m, 1H), 1.51 (d, J = 5.5 Hz, 1H), 1.42 (t, J = 6.5 Hz, 1H), 1.39-1.27 (m, 2H), 1.10-1.03 (m, 2H), 0.77 (t, J = 7.3 Hz, 3H). |
| 109 | 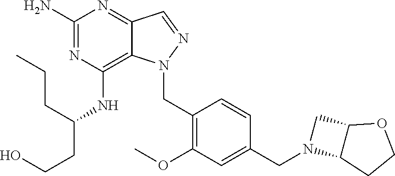 [SMILES: CCC[C@@H](CCO)Nc1nc(N)nc2cnn(Cc3ccc(CN4C[C@@H]5OCC[C@@H]54)cc3OC)c12] (3S)-3-({5-amino-1-[(2-methoxy-4- {[(1S,5S)-2-oxa-6-azabicyclo- [3.2.0]heptan-6-yl]methyl}phenyl)- methyl]-1H-pyrazolo[4,3-d] pyrimidin-7-yl}amino)hexan-1-ol | 2.2 | | LC/MS [M + H]⁺ = 482.3 RT (min) = 0.79 (Procedure B) δ 7.58 (s, 1H), 6.99 (s, 1H), 6.76 (d, J = 7.9 Hz, 1H), 6.39 (d, J = 7.5 Hz, 1H), 5.73 (d, J = 8.7 Hz, 1H), 5.70-5.63 (m, 3H), 5.55 (d, J = 16.9 Hz, 1H), 4.63 (q, J = 4.7 Hz, 1H), 4.33 (d, J = 9.0 Hz, 1H), 4.10-3.98 (m, 2H), 3.96 (s, 1H), 3.85 (s, 3H), 3.65 (s, 1H), 3.33 (t, J = 6.8 Hz, 1H), 3.22-3.11 (m, 2H), 1.92 (s, 2H), 1.73-1.60 (m, 2H), 1.52 (t, J = 7.0 Hz, 1H), 1.37 (dt, J = 26.6, 8.1 Hz, 3H), 1.05 (s, 2H), 0.76 (t, J = 7.3 Hz, 3H) |
| 110 | 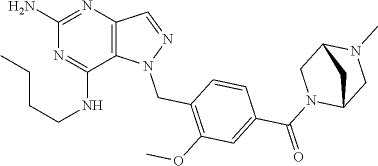 [SMILES: CCCCNc1nc(N)nc2cnn(Cc3ccc(C(=O)N4C[C@H]5C[C@@H]4CN5C)cc3OC)c12] N7-butyl-1-({2-methoxy-4-[(1R,4R)- 5-methyl-2,5-diazabicyclo- [2.2.1]heptane-2-carbonyl]- phenyl}methyl)-1H-pyrazolo[4,3- d]pyrimidine-5,7-diamine | 377.9 | 2.0 | LC/MS [M + H]⁺ = 465.1 RT (min) = 1.28 (Procedure A) ¹H NMR (400 MHz, DMSO-d₆) δ 7.58 (s, 1H), 7.19-7.06 (m, 1H), 7.02-6.87 (m, 1H), 6.54-6.47 (m, 1H), 6.41 (dd, J = 7.8, 2.8 Hz, 1H), 5.67 (br s, 2H), 5.65 (br s, 2H), 4.59-4.04 (m, 1H), 3.88 (s, 3H), 3.47- 3.36 (m, 4H), 2.83-2.54 (m, 2H), 2.36- 2.21 (m, 3H), 1.87-1.58 (m, 2H), 1.47 (quin, J = 7.2 Hz, 2H), 1.26 -1.11 (m, 2H), 0.83 (t, J = 7.4 Hz, 3H) |
| 111 | 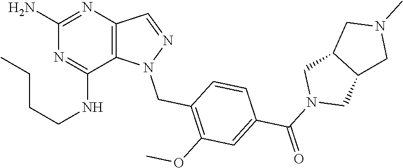 [SMILES: CCCCNc1nc(N)nc2cnn(Cc3ccc(C(=O)N4C[C@H]5CN(C)C[C@H]5C4)cc3OC)c12] 1-({4-[(3aR,6aS)-5-methyl- octahydropyrrolo[3,4-c]pyrrole-2- carbonyl]-2-methoxyphenyl}- methyl)-N7-butyl-1H-pyrazolo[4,3- d]pyrimidine-5,7-diamine | 16.4 | 11.1 | LC/MS[M+H]+ = 479.0 RT (min) = 1.21 (Procedure A) ¹H NMR (400 MHz, DMSO-d₆) δ 7.58 (s, 1H), 7.09 (d, J = 1.1 Hz, 1H), 6.91 (dd, J = 7.7, 1.3 Hz, 1H), 6.50 (br t, J = 5.2 Hz, 1H), 6.40 (d, J = 7.7 Hz, 1H), 5.70-5.62 (m, 4H), 3.88 (s, 3H), 3.75 (br s, 1H), 3.56 (br s, 1H), 3.45- 3.33 (m, 3H), 3.19 (br s, 1H), 2.75 (br s, 2H), 2.44 (br s, 2H), 2.33 (br s, 2H), 2.20 (s, 3H), 1.48 (quin, J = 7.3 Hz, 2H), 1.19 (sxt, J = 7.4 Hz, 2H), 0.83 (t, J = 7.4 Hz, 3H) |
| 112 | 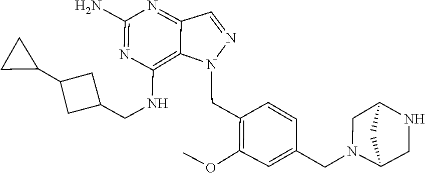 [SMILES: COc1cc(CN2C[C@@H]3C[C@H]2CN3)ccc1Cn1ncc2nc(N)nc(NCC3CC(C4CC4)C3)c21] N7-[(3-cyclopropylcyclobutyl)- methyl]-1-[(4-{[(1S,4S)-2,5- diazabicyclo[2.2.1]heptan-2- yl]methyl}-2-methoxy- phenyl)methyl]-1H-pyrazolo[4,3- d]pyrimidine-5,7-diamine | 30.5 | 151.4 | LC/MS [M + H]⁺ = 489.2 RT (min) = 1.18 (Procedure B) δ 7.59 (s, 1H), 7.03 (s, 1H), 6.80 (d, J = 7.8 Hz, 1H), 6.42 (d, J = 7.8 Hz, 2H), 5.69- 5.61 (m, 5H), 3.87 (d, J = 3.3 Hz, 3H), 3.39 (dd, J = 13.4, 7.5 Hz, 2H), 3.15 (d, J = 10.4 Hz, 1H), 2.81 (d, J = 10.2 Hz, 1H), 2.74 (d, J = 10.1 Hz, 1H), 2.49 (d, J = 10.0 Hz, 1H), 2.33-2,26 (m, 1H), 1.95-1.74 (m, 6H), 1.66-1.60 (m, 2H), 1.54 (d, J = 9.8 Hz, 1H), 1.26 (q, J = 9.8 Hz, 2H), 0.84 (d, J = 7.6 Hz, 1H), 0.69 (d, J = 5.5 Hz, 1H), 0.40- 0.29 (m,3H) |
| 113 | 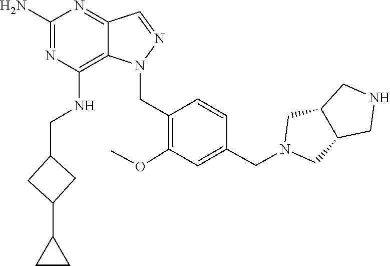 [SMILES: COc1cc(CN2C[C@H]3CNC[C@H]3C2)ccc1Cn1ncc2nc(N)nc(NCC3CC(C4CC4)C3)c21] 1-[(4-{[(3aR,6aS)-octahydro- pyrrolo[3,4-c]pyrrol-2-yl]methyl}-2- methoxyphenyl)methyl]-N7-[(3- cyclopropylcyclobutyl)methyl]-1H- pyrazolo[4,3-d]pyrimidine-5,7- diamine | 22.2 | 43.2 | LC/MS [M + H]⁺ = 503.2 RT (min) = 1.14 (Procedure B) δ 8.35 (s, 1H), 8.31 (s, 1H), 7.94 (s, 0H), 7.77 (d, J = 2.6 Hz, 1H), 7.17 (d, J = 12.0 Hz, 1H), 6.96 (s, 1H), 6.82 (dd, J = 13.1, 7.7 Hz, 1H), 5.74 (s, 3H), 4.25 (s, 1H), 3.78 (d, J = 7.4 Hz, 3H), 3.66 (t, J = 6.7 Hz, 1H), 3.56 (s, 0H), 2.43-2.35 (m, 1H), 1.95 (dd, J = 17.4, 8.9 Hz, 2H), 1.84-1.75 (m, 1H), 1.74 (s, 1H), 1.69 (t, J = 8.8 Hz, 1H), 1.35 (q, J = 9.8 Hz, 2H), 0.83 (d, J = 6.8 Hz, 1H), 0.70 (d, J = 6.4 Hz, 2H), 0.38-0.28 (m, 3H), 0.08-−0.09 (m, 7H) |
| 114 | 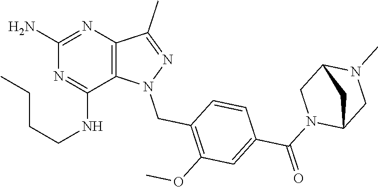 [SMILES: CCCCNc1nc(N)nc2c(C)nn(Cc3ccc(C(=O)N4C[C@H]5C[C@@H]4CN5C)cc3OC)c12] N7-butyl-1-({2-methoxy-4-[(1R,4R)- 5-methyl-2,5-diazabicyclo- [2.2.1]heptane-2-carbonyl]phenyl}- methyl)-3-methyl-1H-pyrazolo[4,3- d]pyrimidine-5,7-diamine | 120.2 | 15.4 | LC/MS [M + H]⁺ = 479.0 RT (min) = 1.28 (Procedure A) δ 7.12 (s, 1H), 7.07 (s, 1H), 6.97 (br d, J = 7.6 Hz, 1H), 6.89 (br d, J = 7.3 Hz, 1H), 6.50 (br s, 1H), 6.46-6.40 (m, 2H), 5.64 (s, 2H), 5.59 (br s, 3H), 3.87 (s, 3H), 3.45- 3.27 (m, 2H), 2.79-2.59 (m, 3H), 2.33 (s, 1H), 2.25 (s, 3H), 1.84 (br d, J = 9.2 Hz, 1H), 1.74 (br d, J = 9.8 Hz, 1H), 1.63 (br d, J = 9.5 Hz, 1H), 1.47 (quin, J = 7.2 Hz, 2H), 1.31- 1.13 (m, 2H), 0.82 (t, J = 7.3 Hz, 3H) |
| 115 | 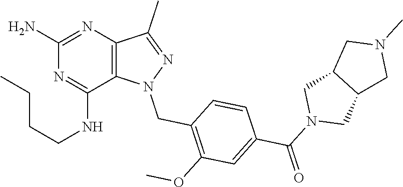 [SMILES: CCCCNc1nc(N)nc2c(C)nn(Cc3ccc(C(=O)N4C[C@H]5CN(C)C[C@H]5C4)cc3OC)c12] 1-({4-[(3aR,6aS)-5-methyl- octahydropyrrolo[3,4-c]pyrrole-2- carbonyl]-2- methoxyphenyl}methyl)-N7-butyl- 3-methyl-1H-pyrazolo[4,3- d]pyrimidine-5,7-diamine | 79.8 | 10.9 | LC/MS [M + H]⁺ = 493.2 RT (min) = 1.25 (Procedure A) δ 7.08 (s, 1H), 6.90 (br d, J = 7.7 Hz, 1H), 6.52 (br s, 1H), 6.38 (br d, J = 7.7 Hz, 1H), 5.70 (br s, 2H), 5.59 (s, 2H), 3.86 (s, 3H), 3.70-3.52 (m 4H), 3.42-3.36 (m, 2H), 2.55 (s, 3H), 2.53-2.21 (m, 10H), 1.91 (s, 2H), 1.46 (quin, J = 7.3 Hz, 2H), 1.31-1.13 (m, 2H), 0.82 (t, J = 7.4 Hz, 3H) |
| 116 | 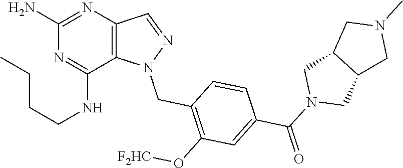 [SMILES: CCCCNc1nc(N)nc2cnn(Cc3ccc(C(=O)N4C[C@H]5CN(C)C[C@H]5C4)cc3OC(F)F)c12] 1-({4-[(3aR,6aS)-5-methyl-octa- hydropyrrolo[3,4-c]pyrrole-2- carbonyl]-2-(difluoromethoxy)phenyl}- methyl)-N7-butyl-1H-pyrazolo[4,3- d]pyrimidine-5,7-diamine | 172.1 | 14.2 | LC/MS [M + H]⁺ = 515.0 RT (min) = 1.34 (Procedure A) δ 8.44 (br t, J = 5.2 Hz, 1H), 7.81 (s, 1H), 7.43-7.06 (m, 3H), 7.00 (d, J = 7.9 Hz, 1H), 5.86 (s, 2H), 3.52 (br s, 1H), 2.84 (br s, 3H), 1.58 (quin, J = 7.3 Hz, 2H), 1.33-1.19 (m, 2H), 0.87 (t, J = 7.3 Hz, 3H) |
| 117 | 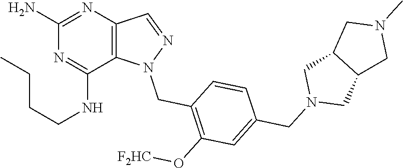 [SMILES: CCCCNc1nc(N)nc2cnn(Cc3ccc(CN4C[C@H]5CN(C)C[C@H]5C4)cc3OC(F)F)c12] 1-[(4-{[(3aR,6aS)-5-methyl-octa- hdropyrrolo[3,4-c]pyrrol-2-yl] methyl}-2-(difluoromethoxy)phenyl)- methyl]-N7-butyl-1H-pyrazolo[4,3- d]pyrimidine-5,7-diamine | 92.4 | 16.2 | LC/MS [M + H]⁺ = 501.0 RT (min) = 1.39 (Procedure A) δ 8.44 (br s, 1H), 7.80 (s, 1H), 7.37-6.96 (m, 3H), 5.84 (s, 2H), 4.19 (br s, 1H), 2.84 (s, 2H), 2.58-2.54 (m, 3H), 1.58 (quin, J = 7.2 Hz, 2H), 1.31-1.20 (m, 2H), 0.88 (t, J = 7.3 Hz, 3H) |
| 118 | 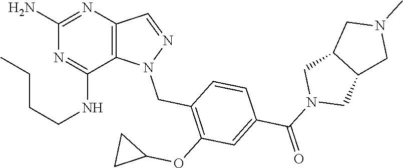 [SMILES: CCCCNc1nc(N)nc2cnn(Cc3ccc(C(=O)N4C[C@H]5CN(C)C[C@H]5C4)cc3OC3CC3)c12] 1-({4-[(3aR,6aS)-5-methyl-octa- hydropyrrolo[3,4-c]pyrrole-2- carbonyl]-2-cyclopropoxyphenyl}- methyl)-N7-butyl-1H-pyrazolo[4,3- d]pyrimidine-5,7-diamine | 339.0 | 51.8 | LC/MS [M + H]⁺ = 505.1 RT (min) = 1.29 (Procedure A) δ 7.58 (s, 1H), 7.35 (s, 1H), 6.95 (br d, J = 7.3 Hz, 1H), 6.60 (br s, 1H), 6.51 (d, J = 7.6 Hz, 1H), 5.69 (br s, 1H), 5.60 (s, 2H), 3.95 (br s, 2H), 3.53 (br s, 1H), 3.41 (br d, J = 5.2 Hz, 2H), 2.80 (br s, 2H), 2.38 (br s, 1H), 2.27 (s, 3H), 1.53-1.42 (m, 2H), 1.32- 1.11 (m, 2H), 0.88-0.76 (m, 5H), 0.60 (br s, 2H) |
| 119 | 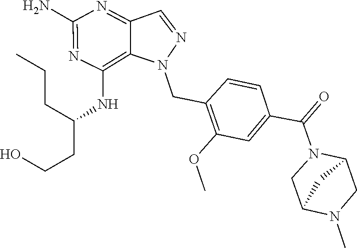 [SMILES: CCC[C@@H](CCO)Nc1nc(N)nc2cnn(Cc3ccc(C(=O)N4C[C@H]5C[C@@H]4CN5C)cc3OC)c12] (3S)-3-{[5-amino-1-({2-methoxy-4- [(1R,4R)-5-methyl-2,5-diazabicyclo [2.2.1]heptane-2-carbonyl] phenyl}methyl)-1H-pyrazolo[4,3- d]pyrimidin-7-yl]amino}hexan-1-ol | 105.6 | 18.1 | LC/MS [M + H]⁺ = 508.9 RT (min) = 1.19 (Procedure B) δ 7.62 (s, 1H), 7.19-7.06 (m, 1H), 7.03- 6.85 (m, 1H), 6.40 (br d, J = 7.3 Hz, 1H), 5.98-5.61 (m, 4H), 4.57 (br s, 1H), 4.38- 4.24 (m, 1H), 4.09 (br s, 1H), 3.89 (s, 3H), 3.60 (br d, J = 2.4 Hz, 2H), 3.51-3.24 (m, 4H), 2.87-2.64 (m, 2H), 2.35 (s, 2H), 2.28 (s, 1H), 1.76 (br d, J = 9.8 Hz, 1H), 1.71- 1.58 (m, 2H), 1.60-1.48 (m, 1H), 1.48- 1.32 (m, 2H), 1.16-0.97 (m, 2H), 0.76 (br t, J = 7.3 Hz, 3H). |
| 120 | 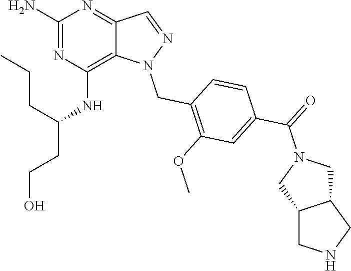 [SMILES: CCC[C@@H](CCO)Nc1nc(N)nc2cnn(Cc3ccc(C(=O)N4C[C@H]5CNC[C@H]5C4)cc3OC)c12] (3S)-3-{[1-({4-[(3aR,6aS)-octa- hydropyrrolo[3,4-c]pyrrole-2-carboxy- phenyl}methyl)-5-amino-1H- pyrazolo[4,3-d]pyrimidin-7- yl]amino}hexan-1-ol | 172.8 | 137.7 | LC/MS [M + H]⁺ = 509.2 RT (min) = 1.04 (Procedure B) δ 7.61 (s, 1H), 7.12 (s, 1H), 6.94 (br d, J = 7.9 Hz, 1H), 6.39 (br d, J = 7.6 Hz, 1H), 5.94-5.61 (m, 4H), 4.32 (br s, 1H), 3.88 (s, 3H), 3.69-3.56 (m, 4H), 3.32 (br d, J = 4.3 Hz, 2H), 3.27-2.96 (m, 4H), 2.94-2.75 (m, 4H), 1.73-1.60 (m, 1H), 1.59-1.49 (m, 1H), 1.48-1.33 (m, 2H), 1.16-0.98 (m, 2H), 0.76 (br t, J = 7.2 Hz, 3H) |
| 121 | 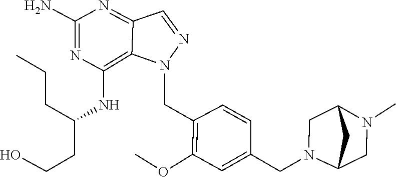 [SMILES: CCC[C@@H](CCO)Nc1nc(N)nc2cnn(Cc3ccc(CN4C[C@H]5C[C@@H]4CN5C)cc3OC)c12] (3S)-3-({5-amino-1-[(2-methoxy-4- {[(1R,4R)-5-methyl-2,5-diaza- bicyclo[2.2.1]heptan-2-yl]methyl}- phenyl)methyl]-1H-pyrazolo[4,3- d]pyrimidin-7-yl}amino)hexan-1-ol | 22.1 | 1.3 | LC/MS [M + H]⁺ = 495.2 RT (min) = 0.98 (Procedure B) δ 7.56 (s, 1H), 7.02 (s, 1H), 6.78 (d, J = 7.8 Hz, 1H), 6.38 (d, J = 7.8 Hz, 1H), 5.68 (d, J = 20.7 Hz, 1H), 5.61 (d, J = 10.5 Hz, 2H), 5.53 (d, J = 17.1 Hz, 1H), 4.29 (s, 1H), 3.83 (s, 2H), 3.74 (s, 5H), 3.36 (s, 1H), 3.33-3.23 (m, 3H), 3.16 (s, 0H), 2.98 (s, 0H), 2.91 (d, J = 10.9 Hz, 1H), 1.85 (s, 3H), 1.71 (s, 2H), 1.62 (dt, J = 13.0, 7.2 Hz, 1H), 1.48 (dd, J = 13.2, 7.9 Hz, 1H), 1.40 (d, J = 8.5 Hz, 1H), 1.32 (t, J = 7.5 Hz, 1H), 1.01 (d, J = 8.1 Hz, 2H), 0.73 (t, J = 7.3 Hz, 3H) |
| 122 | 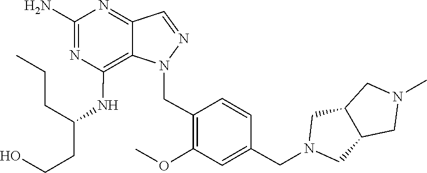 [SMILES: CCC[C@@H](CCO)Nc1nc(N)nc2cnn(Cc3ccc(CN4C[C@H]5CN(C)C[C@H]5C4)cc3OC)c12] (3S)-3-({1-[(4-{[(3aR,6aS)-5-methyl- octahydropyrrolo[3,4-c]pyrrol-2- yl]methyl}-2-methoxyphenyl) methyl]-5-amino-1H-pyrazolo[4,3- d]pyrimidin-7-yl}amino)hexan-1-ol | 7.2 | 0.7 | LC/MS [M + H]⁺ = 509.2 RT (min) = 0.95 (Procedure B) δ 7.80 (s, 1H), 7.62 (d, J = 8.4 Hz, 1H), 7.19 (s, 1H), 6.97 (d, J = 7.7 Hz, 1H), 6.71 (d, J = 7.7 Hz, 1H), 5.83-5.71 (m, 2H), 4.54 (d, J = 8.6 Hz, 1H), 4.19 (s, 0H), 3.81 (s, 3H), 3.60 (s, 1H), 3.39 (t, J = 6.4 Hz, 1H), 2.92 (dt, J = 10.0, 5.4 Hz, 2H), 2.83 (s, 3H), 2.56 (s, 5H), 1.73 (t, J = 6.6 Hz, 2H), 1.53 (d, J = 7.7 Hz, 2H), 1.16 (dt, J = 13.1, 7.2 Hz, 5H), 0.82 (t, J = 7.4 Hz, 3H) |
| 123 | 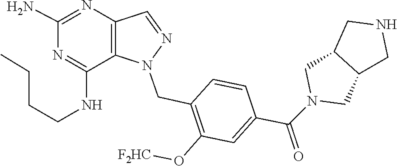 [SMILES: CCCCNc1nc(N)nc2cnn(Cc3ccc(C(=O)N4C[C@H]5CNC[C@H]5C4)cc3OC(F)F)c12] 1-({4-[(3aR,6aS)-octahydro- pyrrolo[3,4-c]pyrrole-2-carbonyl]-2- (difluoromethoxy)phenyl}methyl)- N7-butyl-1H-pyrazolo[4,3- d]pyrimidine-5,7-diamine | 2,238.1 | 481.9 | LC/MS [M + H]⁺ = 501.3 RT (min) = 1.28 (Procedure A) δ 8.33 (br s, 1H), 7.63 (s, 1H), 7.53-7.16 (m, 3H), 6.75-6.65 (m, 1H), 6.49 (br d, J = 8.2 Hz, 1H), 5.78 (s, 2H), 5.70 (s, 2H), 3.80-2.97 (m, 3H), 2.86 (br s, 3H), 2.79- 2.68 (m, 1H), 1.55-1.44 (m, 2H), 1.25- 1.14 (m, 2H), 0.84 (br t, J = 7.2 Hz, 3H) |
| 124 | 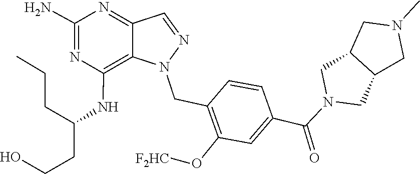 [SMILES: CCC[C@@H](CCO)Nc1nc(N)nc2cnn(Cc3ccc(C(=O)N4C[C@H]5CN(C)C[C@H]5C4)cc3OC(F)F)c12] (3S)-3-{[1-({4-[(3aR,6aS)-5-methyl- octahydropyrrolo[3,4-c]pyrrole-2- carbonyl]-2-(difluoromethoxy)- phenyl}methyl)-5-amino-1H-pyra- zolo[4,3-d]pyrimidin-7-yl]amino}- hexan-1-ol | 82.0 | | LC/MS [M + H]⁺ = 559.0 RT (min) = 1.23 (Procedure A) δ 7.82 (s, 1H), 7.70 (br s, 1H), 7.42-7.05 (m, 3H), 6.81 (br d, J = 7.6 Hz, 1H), 6.03- 5.80 (m, 2H), 4.53 (br s, 1H), 3.80 (s, 1H), 3.58 (br s, 1H), 3.38 (br s, 2H), 3.13 (br d, J = 7.3 Hz, 2H), 2.83 (s, 3H), 1.73 (br d, J = 5.8 Hz, 2H), 1.64-1.47 (m, 2H), 1.21- 1.09 (m, 2H), 0.81 (br t, J = 7.2 Hz, 3H) |
| 125 | 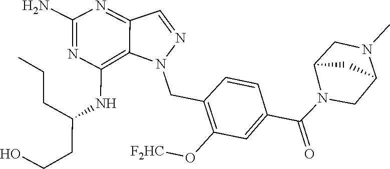 [SMILES: CCC[C@@H](CCO)Nc1nc(N)nc2cnn(Cc3ccc(C(=O)N4C[C@H]5C[C@@H]4CN5C)cc3OC(F)F)c12] (3S)-3-[(5-amino-1-{[2-(difluoro- methoxy)-4-[(1R,4R)-5-methyl- 2,5-diazabicyclo[2.2.1]heptane-2- carbonyl]phenyl]methyl}-1H- pyrazolo[4,3-d]pyrimidin-7- yl)amino]hexan-1-ol | 301 | | LC/MS [M + H]⁺ = 545.4 RT (min) = 1.2 (Procedure A) δ 7.67 (br s, 1H), 7.58-7.18 (m, 3H), 6.52 6.38 (m, 1H), 6.24 (br d, J = 7.9 Hz, 1H), 5.94-5.73 (m, 3H), 4.72-4.14 (m, 2H), 3.33 (br s, 1H), 3.08-2.82 (m, 2H), 2.10- 1.86 (m, 2H), 1.80 (br d, J = 8.5 Hz, 1H), 1.72-1.51 (m, 2H), 1.45 (br s, 2H), 1.09 (br s, 2H), 0.77 (br t, J = 6.6 Hz, 3H) |
| 126 | 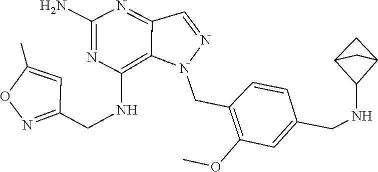 [SMILES: COc1cc(CNC2C3CC2C3)ccc1Cn1ncc2nc(N)nc(NCc3cc(C)on3)c21] 1-({4-[({bicyclo[1.1.1]pentan-1- yl}amino)methyl]-2-methoxy- phenyl}methyl)-N7-[(5-methyl-1,2- oxazol-3-yl)methyl]-1H-pyra- zolo[4,3-d]pyrimidine-5,7-diamine | 287.0 | | LC/MS [M + H]⁺ = 461.1 RT (min) = 0.96 (LC/MS Method 3) δ 7.59-7.55 (m, 1H), 7.28-7.22 (m, 1H), 6.98 (s, 1H), 6.75 (br d, J = 7.7 Hz, 1H), 6.46 (d, J = 7.4 Hz, 1H), 5.98 (s, 1H), 5.75 (s, 2H), 5.58 (s, 2H), 4.63 (br d, J = 1.9 Hz, 2H), 3.73 (s, 3H), 3.62 (br s, 2H), 3.16 (s, 1H), 2.32 (s, 3H), 2.30-2.27 (m, 1H), 1.65 (s, 6H) |
| 127 | 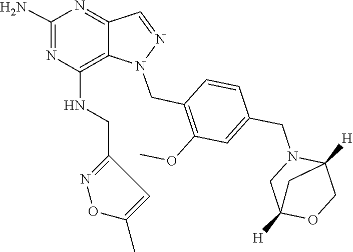 [SMILES: COc1cc(CN2C[C@@H]3C[C@H]2CO3)ccc1Cn1ncc2nc(N)nc(NCc3cc(C)on3)c21] 1-[(2-methoxy-4-{[(1S,4S)-2-oxa-5- azabicyclo[2.2.1]heptan-5-yl] methyl}phenyl)methyl]-N7-[(5- methyl-1,2-oxazol-3-yl)methyl]-1H- pyrazolo[4,3-d]pyrimidine-5,7- diamine | 682. | 16.7 | LC/MS [M + H]⁺ = 477.1 RT (min) = 1.17 (LC/MS Method 3) δ 7.58-7.55 (m, 1H), 7.36-7.29 (m, 1H), 7.01 (s, 1H), 6.80 (br d, J = 7.4 Hz, 1H), 6.65 (d, J = 7.4 Hz, 1H), 5.66 (s, 2H), 5.58 (s, 2H), 4.77 (br s, 2H), 3.81 (br s, 2H), 3.78 (s, 3H), 3.70 (s, 2H), 3.23 (br t, J = 10.7 Hz, 2H), 2.63-2.57 (m, 1H), 2.55 (s, 3H), 1.75 (br d, J = 12.7 Hz, 2H), 1.32-1.21 (m, 2H) |
| 128 | 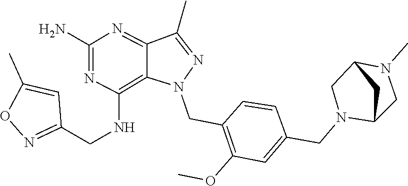 [SMILES: COc1cc(CN2C[C@H]3C[C@@H]2CN3C)ccc1Cn1nc(C)c2nc(N)nc(NCc3cc(C)on3)c21] 1-[(2-methoxy-4-{[(1R,4R)-5- methyl-2,5-diazabicyclo[2.2.1]- heptan-2-yl]methyl}phenyl)- methyl]-3-methyl-N7-[(5-methyl- 1,2-oxazol-3-yl)methyl]-1H-pyra- zolo[4,3-d]pyrimidine-5,7-diamine | 74.4 | 28.0 | [M + H]⁺ = 504.2 LC RT = 1.04 (LC/MS Method 3) δ 6.99 (s, 1H), 6.80 (br d, J = 8.2 Hz, 1H), 6.51 (br d, J = 7.6 Hz, 1H), 6.03 (s, 1H), 5.75 (s, 2H), 5.54 (s, 2H), 4.67 (br d, J = 4.6 Hz, 2H), 3.77 (s, 2H), 3.55 (br s, 2H), 3.36- 3.24 (m, 2H), 2.89 (br d, J = 9.8 Hz, 1H), 2.70- 2.62 (m, 1H), 2.38 (s, 3H), 2.36 (s, 4H), 2.26 (s, 3H), 1.93 (s, 2H), 1.71 (br s, 2H) |
| 129 | 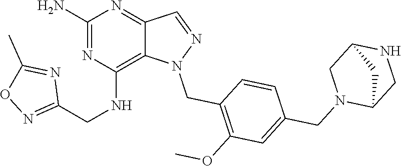 [SMILES: COc1cc(CN2C[C@@H]3C[C@H]2CN3)ccc1Cn1ncc2nc(N)nc(NCc3noc(C)n3)c21] 1-[(4-{[(1S,4S)-2,5-diaza- bicyclo[2.2.1]heptan-2-yl]methyl}-2- methoxyphenyl)methyl]-N7-[(5- methyl-1,2,4-oxadiazol-3-yl) methyl]-1H-pyrazolo[4,3-d]pyri- midine-5,7-diamine | 323 | 804.5 | LC/MS [M + H]⁺ = 477.2 RT (min) = 1.05 (LC/MS Method 3) δ 7.58-7.56 (m, 1H), 7.35-7.29 (m, 1H), 6.95 (s, 1H), 6.80-6.75 (m, 1H), 6.64 (d, J = 7.6 Hz, 1H), 5.66 (s, 2H), 5.58 (s, 2H), 4.77 (br s, 2H), 3.78 (s, 3H), 3.64-3.62 (m, 2H), 3.09 (br d, J = 9.4 Hz, 1H), 2.75 (br d, J = 10.8 Hz, 1H), 2.72-2.67 (m, 1H), 2.55 (br s, 3H), 2.47-2.41 (m, 1H), 1.78-1.74 (m, 1H), 1.49 (br d, J = 9.7 Hz, 1H) |
| 130 | 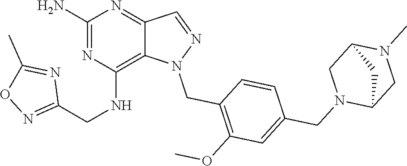 [SMILES: COc1cc(CN2C[C@@H]3C[C@H]2CN3C)ccc1Cn1ncc2nc(N)nc(NCc3noc(C)n3)c21] 1-[(2-methoxy-4-{[(1S,4S)-5- methyl-2,5-diazabicyclo[2.2.1]- heptan-2-yl]methyl}phenyl) methyl]-N7-[(5-methyl-1,2,4-oxa- diazol-3-yl)methyl]-1H-pyrazolo[4,3- d]pyrimidine-5,7-diamine | 134.6 | 74.3 | LC/MS [M + H]⁺ = 491.1 RT(min) = 0.98/Method 3 δ 7.68 (s, 1H), 7.42 (br t, J = 4.8 Hz, 1H), 7.08-7.05 (m, 1H), 6.89 (br d, J = 7.2 Hz, 1H), 6.74 (d, J = 7.7 Hz, 1H), 5.77 (s, 2H), 5.69 (s, 2H), 4.88 (br d, J = 5.2 Hz, 2H), 3.89 (s, 3H), 3.77-3.68 (m, 3H), 3.40-3.34 (m, 2H), 2.76-2.66 (m, 3H), 2.66-2.64 (m, 3H), 2.50 (s, 3H) |
| 131 | 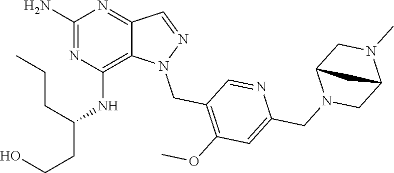 [SMILES: CCC[C@@H](CCO)Nc1nc(N)nc2cnn(Cc3cnc(CN4C[C@@H]5C[C@H]4CN5C)cc3OC)c12] (3S)-3-({5-amino-1-[(4-methoxy-6- {[(1S,4S)-5-methyl-2,5-diaza- bicyclo[2.2.1]heptan-2-yl]methyl} pyridin-3-yl)methyl]-1H-pyrazolo[4,3- d]pyrimidin-7-yl}amino)hexan-1-ol | 333.3 | 11.7 | LC/MS [M + H]⁺ = 496.4 RT (min) = 1.22/Method 4 ¹H NMR (400 MHz, DMSO-d₆) δ 7.57 (s, 1H), 7.47 (s, 1H), 7.10 (s, 1H), 6.09 (d, J = 8.6 Hz, 1H), 5.71-5.55 (m, 4H), 4.40- 4.33 (m, 1H), 3.87 (s,3H), 3.67 (d, J = 5.4 Hz, 2H), 3.40-3.39 (m, 1H), 3.12 (s, 1H), 2.69 (br s, 1H), 2.63-2.59 (m, 2H), 2.26 (s, 3H), 1.79 (s, 2H), 1.70-1.57 (m, 4H), 1.50- 1.43 (m, 2H), 1.13 (br dd, J = 7.7, 3.8 Hz, 2H), 0.80 (t, J = 7.3 Hz, 3H) |
| 132 | 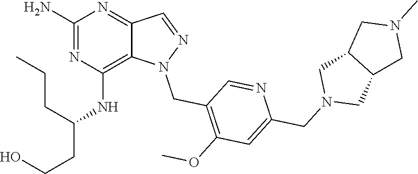 [SMILES: CCC[C@@H](CCO)Nc1nc(N)nc2cnn(Cc3cnc(CN4C[C@H]5CN(C)C[C@H]5C4)cc3OC)c12] (3S)-3-({1-[(6-{[(3aR,6aS)-5-methyl- octahydropyrrolo[3,4-c]pyrrol-2- yl]methyl}-4-methoxypyridin-3- yl)methyl]-5-amino-1H-pyrazolo [4,3-d]pyrimidin-7- yl}amino)hexan-1-ol | 65.9 | 2.2 | LC/MS [M + H]⁺ = 510.4 RT (min) = 1.28/Method 4 ¹H NMR (400 MHz, DMSO-d₆) δ 7.56 (s, 1H), 7.46 (s, 1H), 7.07 (s, 1H), 6.09 (d, J = 8.6 Hz, 1H), 5.71-5.54 (m, 4H), 4.40- 4.30 (m, 1H), 3.86 (s, 4H), 3.55 (d, J = 3.4 Hz, 2H), 2.64-2.53 (m, 4H), 2.47-2.41 (m, 2H), 2.29-2.15 (m, 8H), 1.90 (s, 2H), 1.73-1.58 (m, 2H), 1.52-1.38 (m, 2H), 1.19-1.05 (m, 2H), 0.79 (t, J = 7.2 Hz, 3H) |
| 133 | 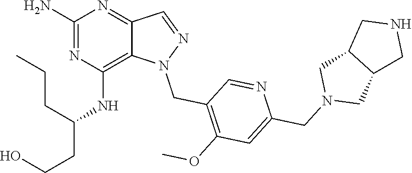 [SMILES: CCC[C@@H](CCO)Nc1nc(N)nc2cnn(Cc3cnc(CN4C[C@H]5CNC[C@H]5C4)cc3OC)c12] (3S)-3-({1-[(6-{[(3aR,6aS)-octa- hydropyrrolo[3,4-c]pyrrol-2-yl] methyl}-4-methoxypyridin-3-yl) methyl]-5-amino-1H-pyrazolo[4,3- d]pyrimidin-7-yl}amino)hexan-1-ol | 89.9 | 2.8 | LC/MS [M + H]⁺ = 496.4 RT(min) = 0.94/Method 4 ¹H NMR (400 MHz, DMSO-d₆) δ 9.86- 9.68 (m, 1H), 8.68 (br s, 1H), 8.47-8.22 (m, 1H), 7.57 (s, 1H), 7.47 (s, 1H), 7.09 (s, 1H), 6.12 (br d, J = 8.6 Hz, 1H), 5.77-5.56 (m, 4H), 4.37 (br dd, J = 6.4, 12.5 Hz, 1H), 3.88-3.85 (m, 3H), 3.57 (s, 4H), 3.39 (br s, 4H), 3.09 (br d, J = 4.4 Hz, 2H), 2.76-2.65 (m, 5H), 2.46 (br s, 4H), 2.08 (s, 2H), 1.74- 1.60 (m, 2H), 1.51-1.43 (m, 2H), 1.18- 1.10 (m, 2H), 0.80 (t, J = 7.3 Hz, 3H) |
| 134 | 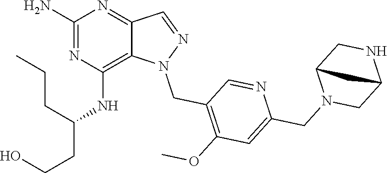 [SMILES: CCC[C@@H](CCO)Nc1nc(N)nc2cnn(Cc3cnc(CN4C[C@@H]5C[C@H]4CN5)cc3OC)c12] (3S)-3-({5-amino-1-[(6-{[(1S,4S)- 2,5-diazabicyclo[2.2.1]heptan-2- yl]methyl}-4-methoxypyridin-3- yl)methyl]-1H-pyrazolo[4,3- d]pyrimidin-7-yl}amino)hexan-1-ol | 353.5 | 22.4 | LC/MS [M + H]⁺ = 482.4 RT (min) = 0.94/Method 4 ¹H NMR (400 MHz, DMSO-d₆) δ 7.56 (d, J = 1.0 Hz, 1H), 7.48 (s, 1H), 7.06 (s, 1H), 6.12 (br d, J = 8.6 Hz, 1H), 5.72-5.53 (m, 5H), 4.41-4.32 (m, 2H), 3.96 (br s, 1H), 3.86 (s, 3H), 3.73 (br d, J = 7.8 Hz, 3H), 3.52-3.51 (m, 2H), 3.39-3.38 (m, 4H), 3.27-3.24 (m, 2H), 2.96-2.91 (m, 1H), 2.84 (br dd, J = 2.3, 10.6 Hz, 1H), 2.60 (br d, J = 10.5 Hz, 1H), 1.95-1.86 (m, 1H), 1.73-1.55 (m, 4H), 1.52-1.41 (m, 2H), 1.19-1.08 (m, 2H), 0.79 (t, J = 7.3 Hz, 3H) |
| 135 | 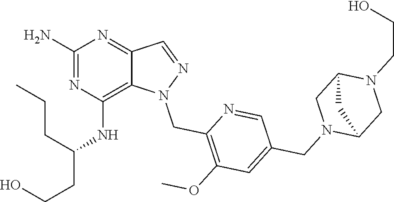 [SMILES: CCC[C@@H](CCO)Nc1nc(N)nc2cnn(Cc3ncc(CN4C[C@@H]5C[C@H]4CN5CCO)cc3OC)c12] (3S)-3-({5-amino-1-[(5-{[(1S,4S)-5- (2-hydroxyethyl)-2,5-diaza- bicyclo[2.2.1]heptan-2-yl]methyl}-3- methoxypyridin-2-yl)methyl]-1H- pyrazolo[4,3-d]pyrimidin-7- yl}amino)hexan-1-ol | 296.6 | 12.2 | LC/MS [M + H]⁺ = 526.4 RT (min) = 1.01/Method 4 ¹H NMR (400 MHz, DMSO-d₆) δ 12.25 (br d, J = 2.2 Hz, 1H), 9.21 (dt, J = 1.7, 4.2 Hz, 2H), 8.01 (s, 1H), 7.85-7.79 (m, 1H), 7.73 (s, 1H), 7.56-7.43 (m, 1H), 5.79 (s, 2H), 4.62-4.50 (m, 2H), 4.34-4.25 (m, 1H), 3.91 (s, 3H), 3.72-3.65 (m, 3H), 3.47 (br s, 2H), 3.17 (s, 3H), 2.07 (br d, J = 1.0 Hz, 1H), 1.84-1.76 (m, 2H), 1.66-1.59 (m, 2H), 1.33-1.22 (m, 2H), 0.88 (t, J = 7.2 Hz, 3H) |
| 136 | 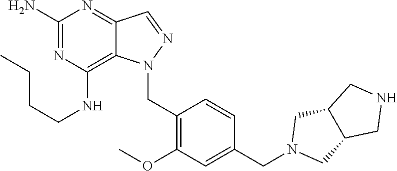 [SMILES: CCCCNc1nc(N)nc2cnn(Cc3ccc(CN4C[C@H]5CNC[C@H]5C4)cc3OC)c12] 1-[(4-{[(3aR,6aS)-octahydro- pyrrolo[3,4-c]pyrrol-2-yl]methyl}-2- methoxyphenyl)methyl]-N7-butyl- 1H-pyrazolo[4,3-d]pyrimidine-5,7- diamine | 62.2 | 1.3 | |
| 137 | 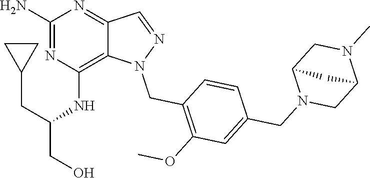 [SMILES: COc1cc(CN2C[C@H]3C[C@@H]2CN3C)ccc1Cn1ncc2nc(N)nc(N[C@H](CO)CC3CC3)c21] (2S)-2-({5-amino-1-[(2-methoxy-4- {[(1R,4R)-5-methyl-2,5-diaza- bicyclo[2.2.1]heptan-2-yl]methyl}- phenyl]methyl]-1H-pyrazolo[4,3- d]pyrimidin-7-yl}amino)-3- cyclopropylpropan-1-ol | 575.5 | 13.2 | LC/MS [M + H]⁺ = 493.2 RT (min) = 1.13 min/Method 3 δ 7.65-7.46 (m, 1H), 7.10-6.93 (m, 1H), 6.86-6.69 (m, 1H), 6.48 (br d, J = 7.6 Hz, 1H), 5.81-5.58 (m, 3H), 5.55-5.37 (m, 1H), 4.36-4.12 (m, 1H), 3.84 (s, 2H), 3.73- 3.51 (m, 2H), 3.50-3.26 (m, 2H), 2.79- 2.58 (m, 3H), 1.94-1.86 (m, 3H), 1.79 (br s, 2H), 1.47-1.14 (m, 3H), 0.55-0.36 (m, 1H), 0.22 (br d, J = 6.4 Hz, 2H), −0.09 (br d, J = 4.9 Hz, 2H). |
| 138 | 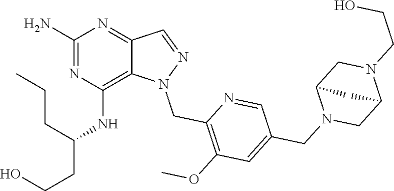 [SMILES: CCC[C@@H](CCO)Nc1nc(N)nc2cnn(Cc3ncc(CN4C[C@H]5C[C@@H]4CN5CCO)cc3OC)c12] (3S)-3-({5-amino-1-[(5-{[(1R,4R)-5- (2-hydroxyethyl)-2,5-diaza- bicyclo[2.2.1]heptan-2-yl]methyl}-3- methoxypyridin-2-yl)methyl]-1H- pyrazolo[4,3-d]pyrimidin-7- yl}amino)hexan-1-ol | 278.0 | 3.0 | LC/MS [M + H]⁺ = 526.4 RT (min) = 0.99/Method 4 ¹H NMR (400 MHz, DMSO-d₆) δ 8.02 (s, 1H), 7.82 (d, J = 8.6 Hz, 1H), 7.47 (s, 1H), 7.46 (s, 1H), 5.63 (d, J = 2.0 Hz, 2H), 5.57 (s, 2H), 4.44-4.36 (m, 1H), 3.88 (s, 3H), 3.75- 3.70 (m, 2H), 3.68-3.63 (m, 2H), 3.46 (br s, 3H), 3.24-3.22 (m, 3H), 2.78-2.62 (m, 5H), 2.58-2.53 (m, 2H), 1.91 (s, 4H), 1.82- 1.54 (m, 6H), 1.37-1.25 (m, 2H), 0.87 (t, J = 7.2 Hz, 3H) |
| 139 | 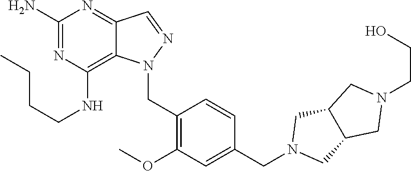 [SMILES: CCCCNc1nc(N)nc2cnn(Cc3ccc(CN4C[C@H]5CN(CCO)C[C@H]5C4)cc3OC)c12] 2-[(3aR,6aS)-5-[(4-{[5-amino-7- (butylamino)-1H-pyrazolo[4,3-d] pyrimidin-1-yl]methyl}-3-methoxy- pheny)methyl]-octahydropyrrolo[3,4- c]pyrrol-2-yl]ethan-1-ol | 19.6 | 1.9 | [M + H]⁺ = 494.9 LC RT = 1.36 (LC/MS Method A) ¹H NMR (500 MHz, DMSO-d₆) δ 8.32 (s, 2H), 7.94 (s, 2H), 7.78 (s, 2H), 7.29 (s, 1H), 7.19 (s, 1H), 7.09 (s, 1H), 6.96 (s, 2H), 6.83 (s, 2H), 5.75 (s, 2H), 3.70 (s, 2H), 3.63- 3.56 (m, 2H), 2.56 (s, 5H), 1.60 (q, J = 7.3 Hz, 4H), 1.28 (q, J = 7.6 Hz, 4H), 0.90 (t, = 7.4 Hz, 6H). |
| 140 | 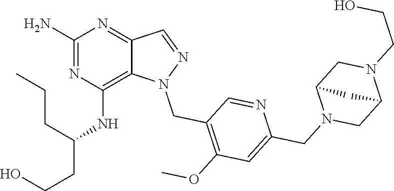 [SMILES: CCC[C@@H](CCO)Nc1nc(N)nc2cnn(Cc3cnc(CN4C[C@H]5C[C@@H]4CN5CCO)cc3OC)c12] (2-hydroxyethyl)-2,5-diazabicyclo- [2.2.1]heptan-2-yl]methyl}-4- methoxypyridin-3-yl)methyl]-1H- pyrazolo[4,3-d]pyrimidin-7- yl}amino)hexan-1-ol | 716.1 | 31.6 | LC/MS [M + H]⁺ = 526.4 RT (min) = 0.94/Method 4 ¹H NMR (400 MHz, DMSO-d₆) δ 9.06-8.82 (m, 2H), 8.53-8.22 (m, 2H), 7.66-7.45 (m, 1H), 7.10 (br s, 1H), 6.51-6.27 (m, 1H), 6.06-5.84 (m, 1H), 5.74-5.55 (m, 1H), 4.55-4.34 (m, 1H), 3.87 (s, 2H), 3.65 (br s, 1H), 3.49-3.38 (m, 3H), 3.17-2.82 (m, 3H), 2.30-2.07 (m, 3H), 2.00 (br s, 1H), 1.75-1.60 (m, 1H), 1.55-1.42 (m, 1H), 1.20-1.06 (m, 4H), 0.81 (br t, J = 7.3 Hz, 1H) |
| 141 | 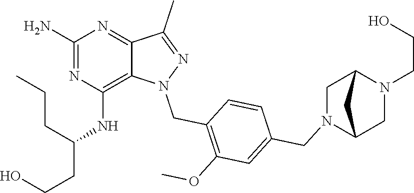 [SMILES: CCC[C@@H](CCO)Nc1nc(N)nc2c(C)nn(Cc3ccc(CN4C[C@H]5C[C@@H]4CN5CCO)cc3OC)c12] (3S)-3-({5-amino-1-[(4-{[(1R,4R)-5- (2-hydroxyethyl)-2,5-diazabicyclo [2.2.1]heptan-2-yl]methyl}-2- methoxyphenyl)methyl]-3-methyl- 1H-pyrazolo[4,3-d]pyrimidin-7- yl}amino)hexan-1-ol | 59.9 | 10.6 | LC/MS [M + H]⁺ = 539.4 RT (min) = 1.04/Method 4 ¹H NMR (400 MHz, DMSO-d₆) δ = 7.01 (s, dH), 6.78 (d, J = 8.8 Hz, 1H), 6.39 (d, J = 7.8 Hz, 1H), 5.70-5.53 (m, 4H), 5.50-5.40 (m, 1H), 3.85 (s, 3H), 3.67-3.55 (m, 2H), 3.42-3.39 (m, 2H), 3.18 (br s, 2H), 2.68 (br s, 1H), 2.58 (br s, 3H), 2.25 (s, 3H), 1.91 (s, 2H), 1.66-1.27 (m, 6H), 1.11-0.96 (m, 2H), 0.75 (t, J = 7.3 Hz, 3H) |
| 142 | 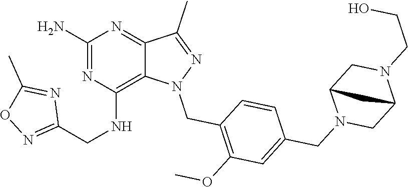 [SMILES: COc1cc(CN2C[C@@H]3C[C@H]2CN3CCO)ccc1Cn1nc(C)c2nc(N)nc(NCc3noc(C)n3)c21] 2-[(1S,4S)-5-({4-[(5-amino-3- methyl-7-{[(5-methyl-1,2,4-oxadiazol- 3-yl)methyl]amino}-1H-pyrazolo- [4,3-d]pyrimidin-1-yl)methyl]-3- methoxyphenyl}methyl)-2,5-diaza- bicyclo[2.2.1]heptan-2-yl]ethan-1-ol | 1232.83 | | LC/MS m/z = 535.2 [M + H]+ RT (min) = 0.768 (LC/MS method 4) ¹H NMR (400 MHz, DMS0-d6) δ = 7.25 (t, J = 5.7 Hz, 1H), 6.96 (s, 1H), 6.78 (d, J = 8.3 Hz, 1H), 6.62 (d, J = 8.1 Hz, 1H), 5.68 (s, 2H), 5.51 (s, 2H), 4.78 (d, J = 5.9 Hz, 2H), 4.41-4.30 (m, 1H), 4.09 (br s, 1H), 3.79 (s, 3H), 3.60 (q, J = 13.8 Hz, 2H), 3.42-3.39 (m, 2H), 3.20-3.16 (m, 2H), 2.71-2.66 (m, 2H), 2.63-2.57 (m, 3H), 2.56 (s, 3H), 2.23 (s, 3H), 1.65-1.50 (m, 2H). |
| 143 | 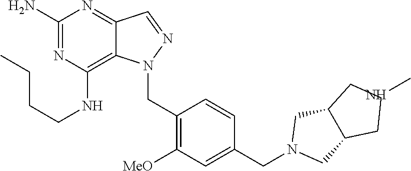 1-[(4-{[(3aR/6aS)-octahydropyrrolo [3,4-c]pyrrol-2-yl]methyl}-2- methoxyphenyl)methyl]-N7-butyl- 1H-pyrazolo[4,3-d]pyrimidine-5,7- diamine | 62.2 | 1.29 | LC/MS [M + H]⁺ = 450.9 RT (min) = 1.28 (LC/MS method A) |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### Industrial Applicability and Uses

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, CP- | CTLA-4 |
| | 675, 206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and analytical (LC/MS) liquid chromatography methods were used:

Analytical LC/MS Procedure A: Column: Waters XBridge C18, 2.1 mm×50 mm, 1.7 μm particles; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0% B to 100% B over 3 min, then a 0.50 min hold at 100% B; Flow: 1 mL/min; Detection: MS and UV (220 nm).

Analytical LC/MS Procedure B: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3 min, then a 0.50 min hold at 100% B; Flow: 1 mL/min; Detection: MS and UV (220 nm).

LC/MS Method 1: Column: BEH C18 2.1×50 mm; Mobile Phase A: water with 0.05% TFA; Mobile Phase B: acetonitrile with 0.05% TFA; Temperature: 50° C.; Gradient: 2-98% B over 1.0 min, then a 0.50 min hold at 98% B; Flow: 0.8 mL/min. Detection: MS and UV (220 nm).

LC/MS Method 2: Column: BEH C18 2.1×50 mm; Mobile Phase A: 95:5 H₂O:acetonitrile with 0.01M NH₄OAc; Mobile Phase B: 5:95 H2O:acetonitrile with 0.01M NH₄OAc; Temperature: 50° C.; Gradient: 5-95% B over 1 min; Flow: 0.8 mL/min.

LC/MS Method 3: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3 min, then a 0.50 min hold at 100% B; Flow: 1 mL/min; Detection: MS and UV (220 nm).

LC/MS Method 4: Column: Waters XBridge BEH C18 XP(50×2.1 mm) 2.5 μm; Mobile Phase A: 5:95 acetonitrile:water with 10 mM NH4OAc; Mobile Phase B: 95:5 acetonitrile:water with 10 mM NH40Ac; Temperature: 50° C.; Gradient: 0-100% B over 3 minutes; Flow: 1.1 ml/min.

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown for convenience, but it is to be understood that they are present in the initial product mixture and separated at a later time, for example by preparative HPLC.

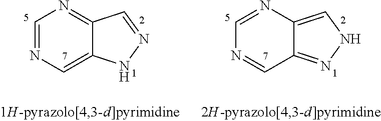 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The compounds of the present disclosure can be prepared by a number of methods well known to one skilled in the art of synthetic organic chemistry. These methods include those described below, or variations thereof. Preferred methods include, but are not limited to, those described below in the Schemes below.

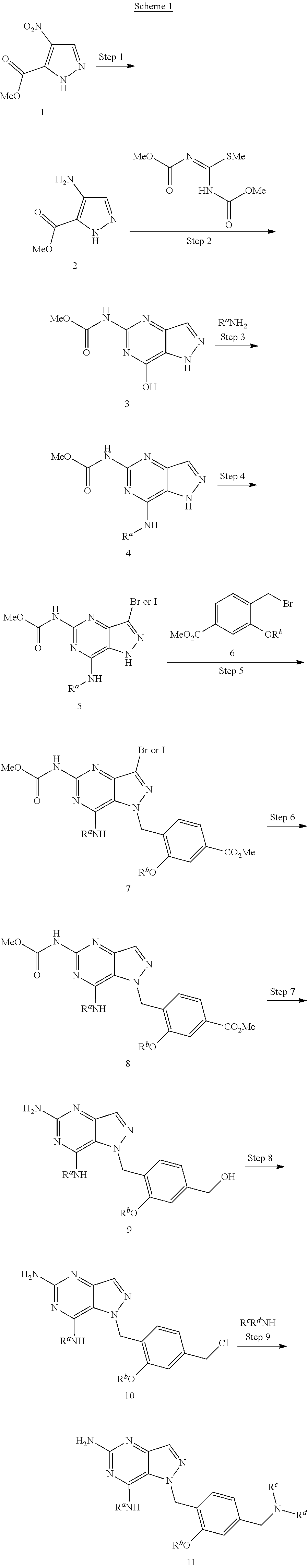

R^{a} can be, in Scheme 1 and other occurrences thereof, for example,

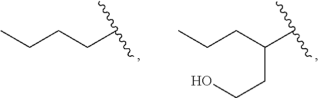

or other suitable moiety. R^{b} is, in Scheme 1 and other occurrences thereof, for example, C₁-C₃ alkyl. R^{c}NHR^{d} is, in Scheme 1 and other occurrences thereof, a primary or secondary amine. R^{a}, R^{b}, R^{c}, and/or R^{d} can have functional groups masked by a protecting group that is removed at the appropriate time during the synthetic process.

Compound 11 can be prepared by the synthetic sequence outlined in Scheme 1 above. Reduction of nitropyrazole 1 to afford compound 2 followed by cyclization with 1,3-bis(methoxycarbonyl)-2-methyl-2-thiopseudourea gives the hydroxypyrazolopyrimidine 3. The amine R^{a}NH₂ is introduced using BOP/DBU coupling conditions, and the subsequent bromination using NBS or iodination using NIS(Step 4) gives the bromo or lodo-pyrazolopyrimidine 5. Alkylation using a benzyl halide 6 gives a mixture of N1 and N2 products, which are separated, giving N1 intermediate 7. Catalytic hydrogenation (step 6) followed by a one-pot LiAlH₄ reduction and carbamate hydrolysis gives the intermediate alcohol 9. Conversion of alcohol 9 to benzyl chloride followed by displacement of it with suitable amines give compound 11. (Alkylation of brominated intermediate 5 in Step 5 gives a better ratio of N1/N2 product, compared to alkylation of unbrominated intermediate 4).

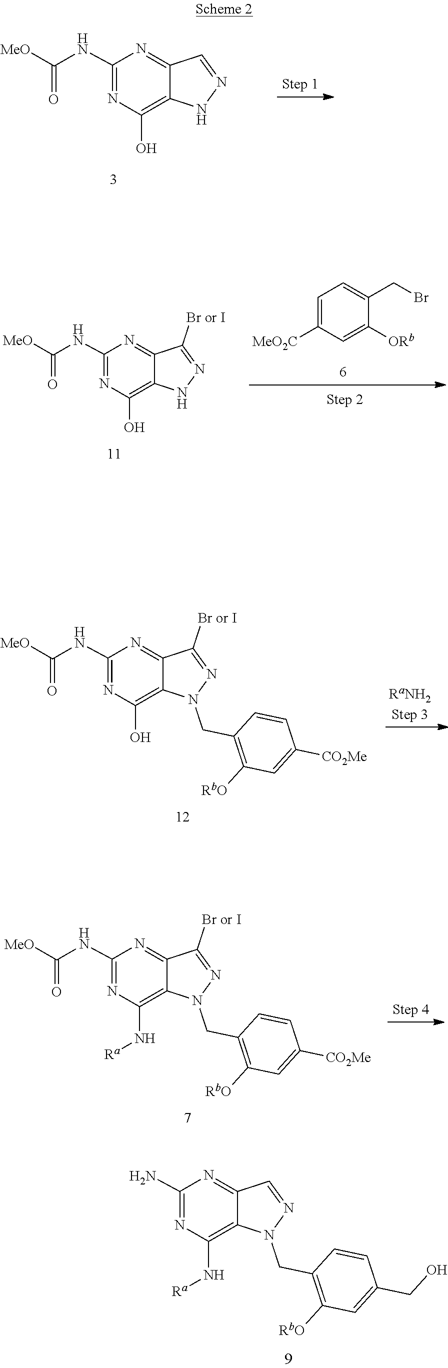

Alternatively, intermediate 9 may be accessed using the route described in Scheme 2 above. Intermediate 3 is brominated or iodinated using NBS or NIS, then alkylated to give the intermediate ester 12. Amination then follows, using BOP coupling conditions to give intermediate 7. Catalytic hydrogenation followed by LiAlH₄ reduction to alcohol and methyl carbamate deprotection gives intermediate 9.

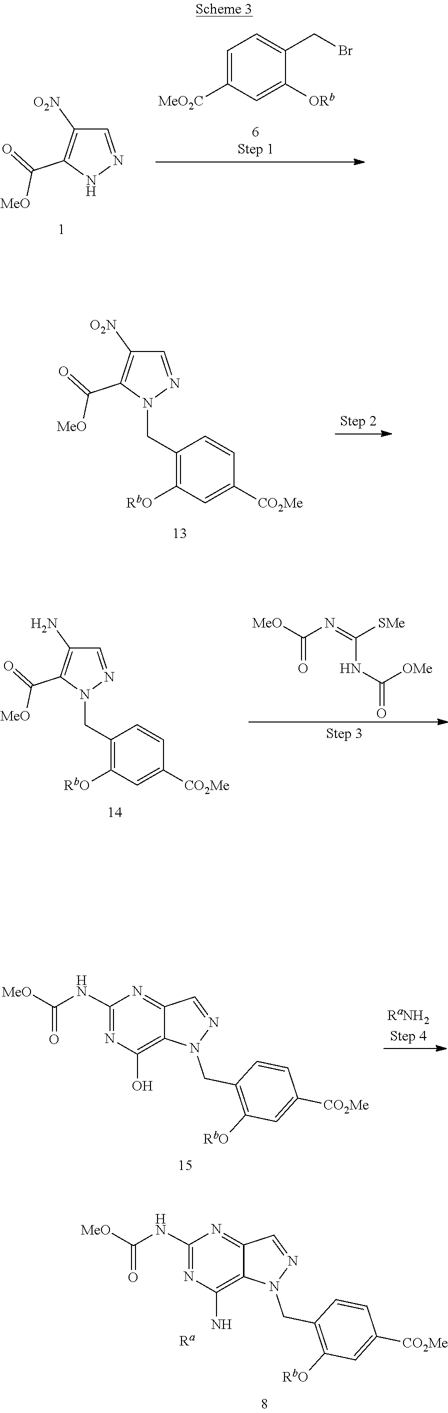

An alternative route to intermediate 8 begins with the alkylation of nitropyrazole 1 with benzyl halide 6, giving the benzyl pyrazole 13. Reduction of the nitro group followed by cyclisation with 1,3-bis(methoxycarbonyl)-2-methyl-2-thiopseudourea gives the hydroxypyrazolopyrimidine 15, which is converted to the appropriate amine derivative 8 using BOP/DBU conditions. This is illustrated in scheme 3 above.

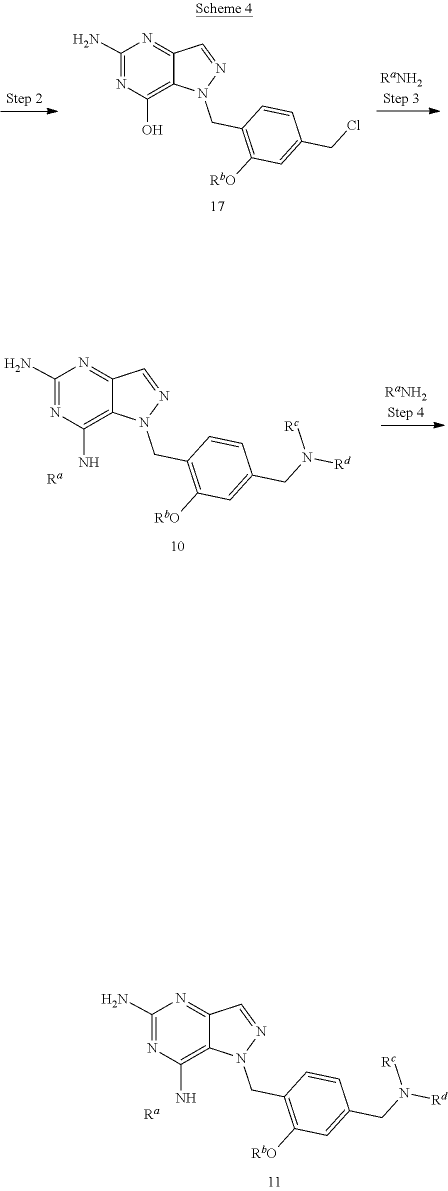

Another alternative route to the target compounds is shown in scheme 4 above. From intermediate 15, the ester group is reduced and the methyl carbamate removed using NaOH, giving the alcohol 16. Conversion of alcohol 16 to chloride followed by displacement with suitable amine gives 17, and subsequent amination using BOP/DBU conditions gives the target molecule 11.

In Scheme 5 below, hydrolysis of methyl ester in 7/8 or 15 followed by amide formation can give corresponding amidnes 7a/8a or 15a. Catalytic hydrogenation of 7a followed by carbamate deprotection produces compound 7b. Carbamate deprotection on 8a gives compound 8b. Finally, amine installation on 15a followed carbamate deprotection gives compound 15b.

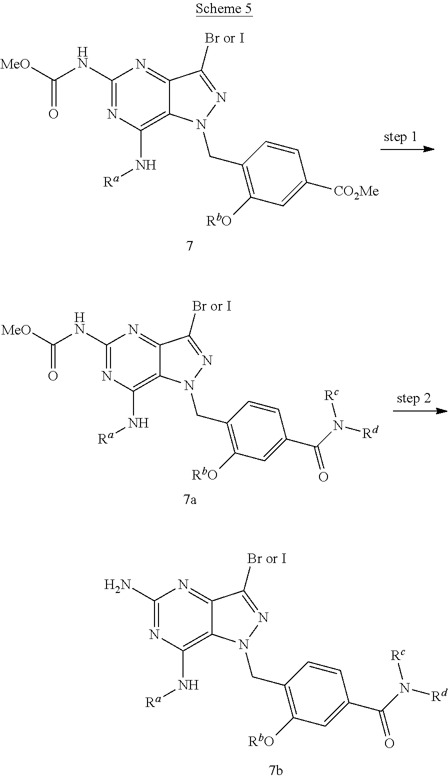

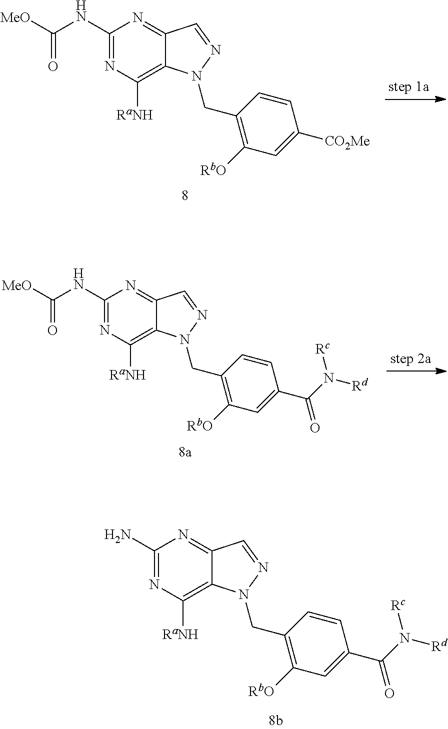

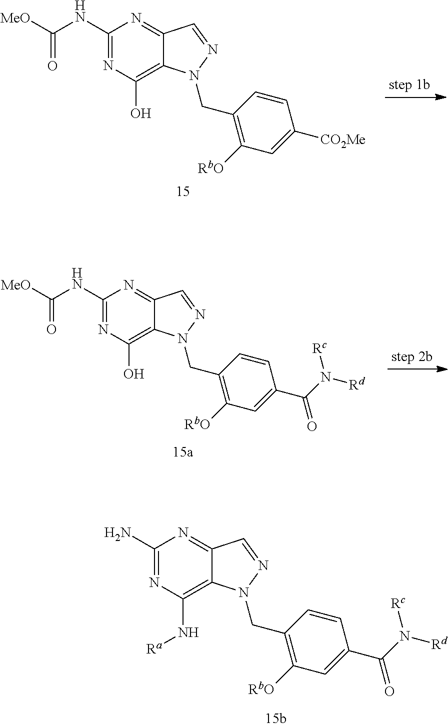

### Synthesis-Specific Examples

To further illustrate the foregoing, the following non-limiting, the following exemplary synthetic schemes are included. Variations of these examples within the scope of the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 101 and higher can be found in Table A.

### Example 1—Compound 101

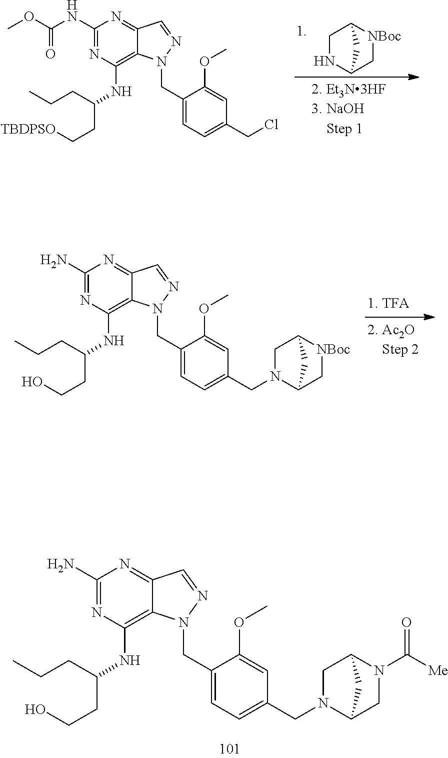

Step 1. A solution of methyl (S)-(7-((1-((tert-butyldiphenylsilyl)oxy)hexan-3-yl)amino)-1-(4-(chloromethyl)-2-methoxybenzyl)-1H-pyrazolo[4,3-d]pyrimidin-5-yl)carbamate (25 mg, 0.035 mmol) was heated with tert-butyl (1S,4S)-2,5-diazabicyclo[2.2.1]heptane-2-carboxylate (35 mg, 0.175 mmol) at 70° C. in 2 mL DMF for 30 min. The base and solvent were evaporated in a V-10 apparatus. The residue was re-dissolved in 2 mL DMF and treated with 3HF·Et₃N. After stirring overnight, the reaction mixture was neutralized with saturated aqueous NaHCO₃. The solvent was evaporated in a V-10 apparatus and purified on a reverse phase ISCO apparatus using acetonitrile/water (0.05% formic acid) on 10 g C-18 column. The solvent was evaporated in a V-10 apparatus and the product was dissolved in 1 mL dioxane and heated with 175 microliter of 1 molar aqueous NaOH solution for 2 h at 70° C. Once the hydrolysis of the carbamate group was completed, the solvent was evaporated in a V-10 apparatus. The residue was dissolved in 2 mL DMF and syringe-filtered. The crude material was purified via preparative LC/MS with the following conditions: Column: XBridge C18, 200 mm×19 mm, 5-μm particles; Mobile Phase A: 5:95 acetonitrile: water with 10-mM NH₄OAc; Mobile Phase B: 95:5 acetonitrile: water with 10-mM NH₄OAc; Gradient: a 0-minute hold at 22% B, 22-62% B over 23 min, then a 4-minute hold at 100% B; Flow Rate: 20 mL/min; Column Temperature: 25° C. Fraction collection was triggered by MS signals. Fractions containing the desired product were combined and dried via centrifugal evaporation to provide 14.7 mg of tert-butyl (1S,4S)-5-(4-((5-amino-7-(((S)-1-hydroxyhexan-3-yl)amino)-1H-pyrazolo[4,3-d]pyrimidin-1-yl)methyl)-3-methoxybenzyl)-2,5-diazabicyclo[2.2.1]heptane-2-carboxylate.

LCMS ESI: calculated for C₃₀H₄₄N₈O₄=580.7 (M+H+), found 580.9 (M+H+).

¹H NMR (500 MHz, DMSO-d6) δ 7.58 (s, 1H), 7.02 (s, 1H), 6.79 (d, J=7.9 Hz, 1H), 6.38 (d, J=7.8 Hz, 1H), 5.68 (d, J=26.6 Hz, 3H), 5.55 (d, J=17.1 Hz, 1H), 4.17 (s, OH), 4.13 (s, 1H), 3.85 (s, 3H), 3.63 (s, 1H), 3.37 (d, J=40.2 Hz, 2H), 3.07 (dd, J=19.7, 10.3 Hz, 1H), 2.79-2.72 (m, 1H), 2.55 (s, 4H), 2.46 (d, J=9.7 Hz, 1H), 2.39 (s, 1H), 1.78 (s, 1H), 1.61 (dd, J=23.0, 10.5 Hz, 2H), 1.50 (d, J=5.5 Hz, 1H), 1.38 (d, J=4.2 Hz, 9H), 1.04 (s, 1H), 0.75 (t, J=7.3 Hz, 3H).

Step 2. A solution of tert-butyl (1S,4S)-5-(4-((5-amino-7-(((S)-1-hydroxyhexan-3-yl)amino)-1H-pyrazolo[4,3-d]pyrimidin-1-yl)methyl)-3-methoxybenzyl)-2,5-diazabicyclo[2.2.1]heptane-2-carboxylate 1 (9.16 mg, 0.016 mmol) in CH₂Cl₂ (0.5 mL) was treated with TFA (0.024 mL, 0.315 mmol). After 30 min, LCMS showed loss of the BOC protecting group. Solvent and TFA were evaporated in a V-10 apparatus. The residue was dissolved in DMF (1 mL), treated with Hunig's base (0.055 mL, 0.315 mmol), followed by Ac₂O (1.5 μL, 0.016 mmol). LCMS showed completion of reaction after 10 min. The base was removed by evaporation. The residue was dissolved in 2 mL DMF. The crude material was purified via preparative LC/MS with the following conditions: Column: XBridge C18, 200 mm×19 mm, 5-um particles; Mobile Phase A: 5:95 acetonitrile: water with NH₄OAc; Mobile Phase B: 95:5 acetonitrile: water with NH₄OAc; Gradient: a 0-minute hold at 6% B, 6-46% B over 20 min, then a 4-minute hold at 100% B; Flow Rate: 20 mL/min; Column Temperature: 25° C. Fraction collection was triggered by MS signals. Fractions containing the desired product were combined and dried via centrifugal evaporation to provide 4.4 mg of Compound 101.

The following compounds were analogously prepared: Compound 107, Compound 108, Compound 109, Compound 121, and Compound 122.

### Example 2—Compound 102

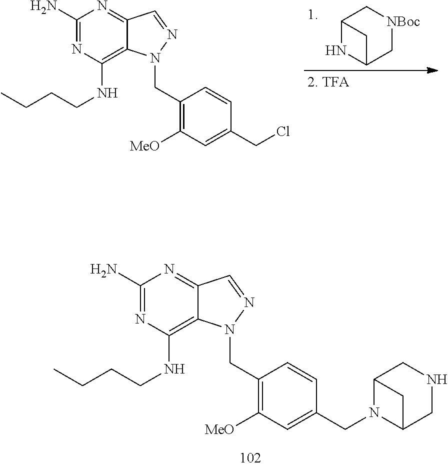 [SMILES: CCCCNc1nc(N)nc2cnn(Cc3ccc(CCl)cc3OC)c12 ; CC(C)(C)OC(=O)N1CC2CC(C1)N2 ; CCCCNc1nc(N)nc2cnn(Cc3ccc(CN4C5CNCC4C5)cc3OC)c12]

A solution of N7-butyl-1-(4-(chloromethyl)-2-methoxybenzyl)-1H-pyrazolo[4,3-d]pyrimidine-5,7-diamine (20 mg, 0,53 mmol) was dissolved in 2 mL DMF and was heated with tert-butyl 3,6-diazabicyclo[3.1.1]heptane-3-carboxylate (53 mg, 0.267 mmol) at 70° C. for 1 h. Excess base was evaporated in a V-10 apparatus. The crude product was treated with 0.082 mL TFA and stirred at RT for 1 h. The TFA was evaporated in a V-10 apparatus and the crude material was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 0% B, 0-40% B over 20 min, then a 4-minute hold at 100% B; Flow Rate: 20 mL/min; Column Temperature: 25° C. Fraction collection was triggered by MS and UV signals. Fractions containing the desired product were combined and dried via centrifugal evaporation to provide 16.2 mg of Compound 102.

The following compounds were analogously prepared: Compound 103, Compound 104, Compound 105, and Compound 143

### Example 3—Compound 112

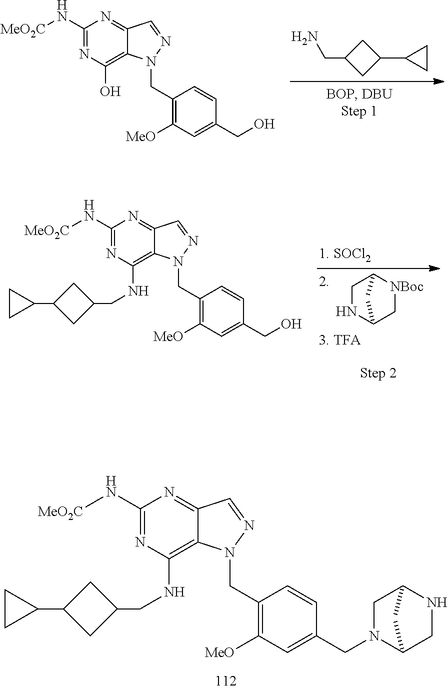

Step 1. A solution of methyl (7-hydroxy-1-(4-(hydroxymethyl)-2-methoxybenzyl)-1H-pyrazolo[4,3-d]pyrimidin-5-yl)carbamate (100 mg, 0.278 mmol) and (3-cyclopropyl-cyclobutyl)methanamine (69.7 mg, 0.557 mmol) in DMSO (2 mL) was treated with DBU (0.126 mL, 0.835 mmol) and BOP (185 mg, 0.417 mmol). After heating at 40° C. for 1 h, NaOH (0.278 mL, 1.391 mmol) was added. The reaction mixture was heated at 80° C. for 2 h and was directly purified on reverse phase ISCO using 50 g C-18 column eluting with 0-50% water/acetonitrile. Product-containing fractions were lyophilized to yield 90 mg of desired product.

LCMS ESI: calculated for C₂₂H₂₈N₆O₂=409.5 (M+H+), found 409.5 (M+H⁺).

Step 2. A solution of (4-((5-amino-7-(((3-cyclopropylcyclobutyl)methyl)amino)-1H-pyrazolo[4,3-d]pyrimidin-1-yl)methyl)-3-methoxyphenyl)methanol (90 mg, 0.220 mmol) in THF (1 mL) was treated with SOCl₂ (0.032 mL, 0.441 mmol) and stirred at RT for 1 h. The solvent was evaporated and the crude chloride product was dissolved in 0.5 mL DMF and heated at 70° C. with tert-butyl (1S,4S)-2,5-diazabicyclo[2.2.1]heptane-2-carboxylate (35 mg, 0.175 mmol) for 1 h. The base was evaporated in a V-10 apparatus and the crude product was treated with 0.5 mL TFA and stirred at RT for 1 h. The TFA was evaporated in a V-10 apparatus and the crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 13% B, 13-53% B over 20 min, then a 0-minute hold at 100% B; Flow Rate: 20 mL/min; Column Temperature: 25° C. Fraction collection was triggered by MS signals. Fractions containing the desired product were combined and dried via centrifugal evaporation to yield 13.2 mg of Compound 112.

Compound 113 was analogously prepared.

### Example 4—Compound 110

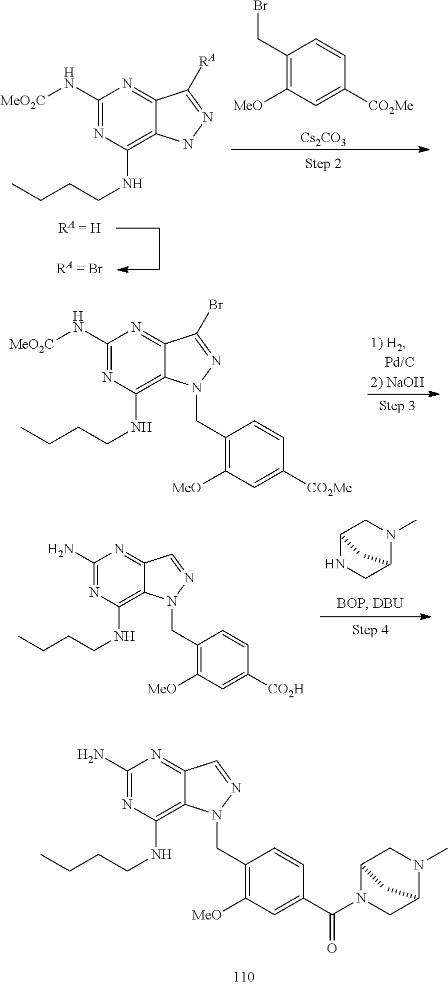

Step 1. A solution of NBS (6.94 g, 39.0 mmol) in DMF (20 mL) was added to a stirred suspension of methyl (7-(butylamino)-1H-pyrazolo[4,3-d]pyrimidin-5-yl)carbamate (10 g, 37.8 mmol) in DMF (80 mL). After stirring at RT for 90 min, the reaction mixture was poured into water (400 mL) and stirred for 5 min. The product was collected by filtration, washed with water (200 mL), and left to air dry overnight, giving methyl (3-bromo-7-(butylamino)-1H-pyrazolo[4,3-d]pyrimidin-5-yl)carbamate (7.5 g, 21.85 mmol, 57.8% yield) as a solid.

LC-MS (ES, m/z): [M+H]⁺=343.0, 345.0.

¹H NMR (400 MHz, DMSO-d₆) δ 12.87 (br s, 1H), 9.80 (s, 1H), 7.56 (br s, 1H), 3.62 (s, 3H), 3.54 (q, J=6.6 Hz, 2H), 1.62 (quin, J=7.2 Hz, 2H), 1.40 (dq, J=14.8, 7.4 Hz, 2H), 0.94 (t, J=7.4 Hz, 3H).

Step 2. A solution of methyl 4-(bromomethyl)-3-methoxybenzoate (1.861 g, 7.18 mmol) in DMF (5 mL) was added portionwise over 5 min to a stirred suspension of methyl (3-bromo-7-(butylamino)-1H-pyrazolo[4,3-d]pyrimidin-5-yl)carbamate (2.9 g, 8.45 mmol) and Cs₂CO₃ (3.30 g, 10.14 mmol) in DMF (35 mL) at 0° C. The reaction mixture was allowed to warm to RT, stirred overnight, poured into saturated NaHCO₃ solution (300 mL), and extracted with EtOAc (3×70 mL). The combined organic phases were washed with brine (4×50 mL), dried (MgSO₄), filtered and concentrated. Flash chromatography (SiO₂ column, 0 to 50% EtOAc in hexanes) gave methyl 4-((3-bromo-7-(butylamino)-5-((methoxycarbonyl)amino)-1H-pyrazolo-[4,3-d]pyrimidin-1-yl)methyl)-3-methoxybenzoate (1.400 g, 2.69 mmol, 31.8% yield) as a solid. LC-MS (ES, m/z): [M+H]⁺=521.2, 523.2.

¹H NMR (400 MHz, DMSO-d₆) δ 9.88 (s, 1H), 7.54-7.48 (m, 2H), 7.32 (t, J=5.6 Hz, 1H), 6.79 (d, J=7.7 Hz, 1H), 5.78 (s, 2H), 3.86 (s, 3H), 3.85 (s, 3H), 3.63 (s, 3H), 3.52 (q, J=6.6 Hz, 2H), 1.56 (quin, J=7.3 Hz, 2H), 1.28-1.15 (m, 2H), 0.84 (t, J=7.4 Hz, 3H).

Step 3. Methyl 4-((3-bromo-7-(butylamino)-5-((methoxycarbonyl)amino)-1H-pyrazolo[4,3-d]pyrimidin-1-yl)methyl)-3-methoxybenzoate (1.400 g, 2.69 mmol) was suspended in EtOH (80 mL). 10% Pd/C (200 mg) was added. The reaction vessel was evacuated and purged with hydrogen six times. The reaction mixture was stirred for 1 h under a hydrogen atmosphere. The reaction vessel was evacuated and purged with nitrogen, then filtered through CELITE™, washing with EtOH (100 mL). The filtrate was evaporated to dryness, leaving a residue, which was dissolved in dioxane (10 mL). NaOH (3.22 mL, 16.11 mmol) was added. The reaction mixture was stirred at 80° C. for 2 h, cooled to RT, diluted with water (10 mL) and acidified with 5N HCl. The dioxane was removed by evaporation. The residue was diluted with more water (20 mL), collected by filtration, washed with water and then acetonitrile, giving 4-((5-amino-7-(butylamino)-1H-pyrazolo[4,3-d]pyrimidin-1-yl)methyl)-3-methoxybenzoic acid (900 mg, 2.430 mmol, 90% yield) as a white solid.

LC-MS (ES, m/z): [M+H]⁺ 371.2.

Step 4. A 20 mL scintillation vial was charged with 4-((5-amino-7-(butylamino)-1H-pyrazolo[4,3-d]pyrimidin-1-yl)methyl)-3-methoxybenzoic acid (160 mg, 0.432 mmol), (1R,4R)-2-methyl-2,5-diazabicyclo[2.2.1]heptane dihydrobromide (100 mg, 0.518 mmol), BOP (210 mg, 0.475 mmol) and DMSO (3 mL). DBU (0.228 mL, 1.512 mmol) was added. The reaction mixture was stirred at 40° C. for 2 h, poured into saturated NaHCO₃ solution (20 mL), and extracted with EtOAc (3×5 mL). The organic phases were discarded. The aqueous layer was evaporated to dryness. The residue was suspended in DCM (5 mL), filtered and purified using flash chromatography (40 g SiO₂ column, 0 to 50% MeOH in DCM), giving Compound 110 (13.2 mg, 0.028 mmol, 6.5% yield) as a solid.

Compound 111 was analogously prepared.

### Example 5—Compound 114

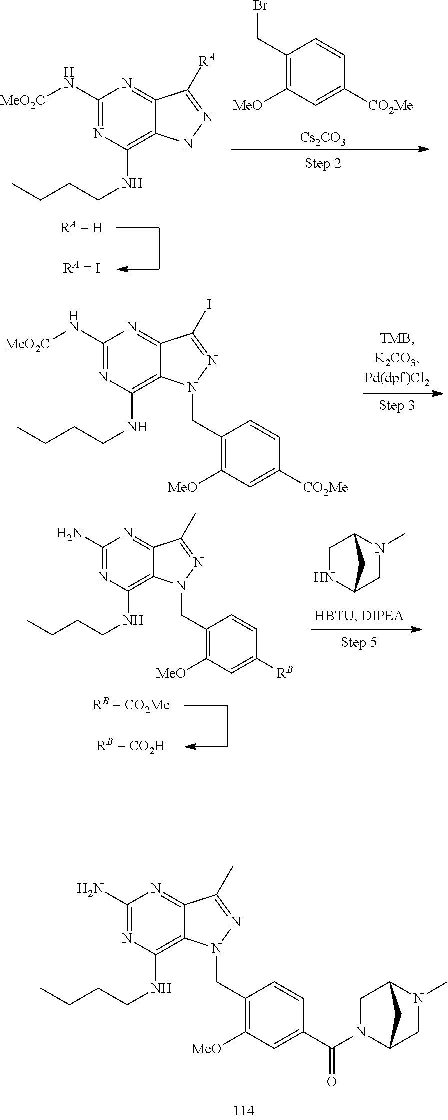

Step 1. A stirred solution of methyl (7-(butylamino)-1H-pyrazolo[4,3-d]pyrimidin-5-yl)carbamate (4.98 g, 18.84 mmol; US 2020/0038403 A1) in DMF (60 mL) was cooled in an ice bath. NIS (5.09 g, 22.61 mmol) was added portion-wise. The reaction mixture was stirred at RT for 2 h and poured into water (400 mL). The precipitate was collected by filtration, giving methyl (7-(butylamino)-3-iodo-1H-pyrazolo[4,3-d]pyrimidin-5-yl)carbamate (6.46 g, 15.73 mmol, 83% yield) as a solid.

LC-MS (ES, m/z): [M+H]⁺=391.1.

¹H NMR (400 MHz, DMSO-d6) δ 12.96 (s, 1H), 9.74 (s, 1H), 7.52 (s, 1H), 3.62 (s, 3H), 3.53 (q, J=6.5 Hz, 2H), 1.68-1.55 (m, 2H), 1.40 (m, 2H), 0.94 (t, J=7.4 Hz, 3H).

Step 2. Cs₂CO₃ (4.18 g, 12.81 mmol) was added to a stirred solution of methyl (7-(butylamino)-3-iodo-1H-pyrazolo[4,3-d]pyrimidin-5-yl)carbamate (2.5 g, 6.41 mmol) in DMF (50 mL). After sonicating for 5 min, a solution of methyl 4-(bromomethyl)-3-methoxybenzoate (1.743 g, 6.73 mmol) in DMF (10 mL) was added. The reaction mixture was stirred at RT for 2 h, poured into 10% citric acid solution (100 mL), and extracted with DCM (3×100 mL). The combined organic phases were washed with brine, dried (Na₂SO₄), filtered and concentrated. Flash chromatography (SiO₂ column, 0 to 100% EtOAc in hexanes) gave methyl 4-((7-(butylamino)-3-iodo-5-((methoxycarbonyl)amino)-1H-pyrazolo[4,3-d]pyrimidin-1-yl)methyl)-3-methoxybenzoate (2.26 g, 2.98 mmol, 46% yield, 75% purity) as a solid, which was used without further purification.

LC-MS (ES, m/z): [M+H]⁺=569.2.

Step 3. A 20 mL microwave vial was charged with methyl 4-((7-(butylamino)-3-iodo-5-((methoxycarbonyl)amino)-1H-pyrazolo[4,3-d]pyrimidin-1-yl)methyl)-3-methoxybenzoate (1.34 g, 1.771 mmol, 75% purity), [1,1′-bis(diphenylphosphino)ferrocene]dichloropalladium(II) (91 mg, 0.124 mmol), trimethylboroxine (1001 mg, 7.97 mmol), K₂CO₃ (734 mg, 5.31 mmol) and dioxane (7 mL). The reaction mixture was heated in a microwave oven at 120° C. for 1 h and diluted with DCM and 10% citric acid. The phases were separated. The organic phase was washed sequentially with 10% citric acid and brine, dried (Na₂SO₄) filtered and concentrated under reduced pressure. Flash chromatography give methyl 4-((5-amino-7-(butylamino)-3-methyl-1H-pyrazolo[4,3-d]pyrimidin-1-yl)methyl)-3-methoxybenzoate (422 mg, 1.06 mmol, 59.8% yield) as a solid.

LC-MS (ES, m/z): [M+H]⁺=399.2.

Step 4. NaOH (1.190 mL, 5.95 mmol) was added to a suspension of methyl 4-((5-amino-7-(butylamino)-3-methyl-1H-pyrazolo[4,3-d]pyrimidin-1-yl)methyl)-3-methoxybenzoate (237 mg, 0.595 mmol) in dioxane (5 mL). After stirring at 80° C. for 1 h, the reaction mixture was cooled, neutralized with 5N hydrochloric acid, and evaporated to dryness. The residue was suspended in DMSO (2 mL) and water (20 mL) and collected by filtration and washed with water, giving 4-((5-amino-7-(butylamino)-3-methyl-1H-pyrazolo[4,3-d]pyrimidin-1-yl)methyl)-3-methoxybenzoic acid (184 mg, 0.479 mmol, 80% yield) as a solid.

LC-MS (ES, m/z): [M−H]⁺=383.2.

Step 5. A 20 mL scintillation vial was charged with 4-((5-amino-7-(butylamino)-3-methyl-1H-pyrazolo[4,3-d]pyrimidin-1-yl)methyl)-3-methoxybenzoic acid (35 mg, 0.091 mmol), 2-(1H-benzotriazol-1-yl)-1,1,3,3-tetramethyluronium hexafluorophosphate (“HBTU,” 41.4 mg, 0.109 mmol), (1R,4R)-2-methyl-2,5-diazabicyclo[2.2.1]heptane dihydrobromide (17.58 mg, 0.091 mmol) and DMF (2 mL). DIPEA (0.048 mL, 0.273 mmol) was added. The reaction mixture was stirred at RT overnight, filtered, and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 4% B, 4-44% B over 20 min, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 114 (15.1 mg, 0.032 mmol, 35% yield).

Compound 115 was analogously prepared.

### Example 6—Compound 116, ditrifluoroacetate

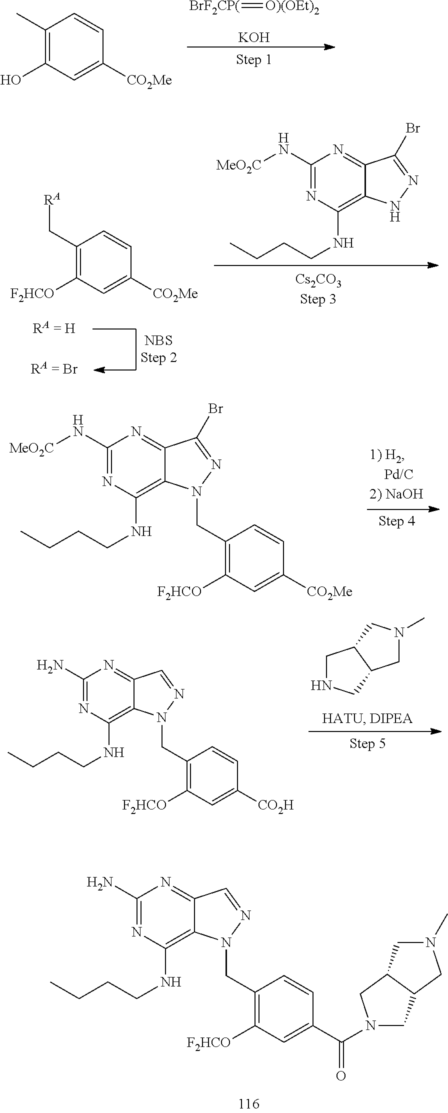

Step 1. A solution of potassium hydroxide (5N, 24.07 mL, 120 mmol) in water was added to a cooled (ice bath) solution of methyl 3-hydroxy-4-methylbenzoate (4 g, 24.07 mmol) in acetonitrile (150 mL). After stirring at 0° C. for 5 min, diethyl (bromodifluoromethyl)-phosphonate (12.85 g, 48.1 mmol) was added. The reaction mixture was allowed to warm slowly to RT and stirred for 16 h. More KOH solution (5N, 16 mL, 80 mmol) was added. The reaction mixture was stirred at RT for a further 30 min, diluted with water (200 mL), and extracted with EtOAc (3×50 mL). The combined organic phases were washed with brine (2×50 mL), dried (MgSO₄), filtered, and concentrated. Flash chromatography (SiO₂ column, 0 to 10% EtOAc in hexanes) gave methyl 3-(difluoromethoxy)-4-methylbenzoate (2.552 g, 11.80 mmol, 49.0% yield) as an oil.

LC-MS (ES, m/z): [M+H]⁺ 217.1.

¹H NMR (400 MHz, DMSO-d₆) δ 7.76 (dd, J=7.8, 1.7 Hz, 1H), 7.68 (br. s, 1H), 7.51-7.10 (m, 2H), 3.87 (s, 3H), 2.31 (s, 3H).

Step 2. NBS (1.811 g, 10.18 mmol) and benzoyl peroxide (0.448 g, 1.850 mmol) were added to a stirred solution of methyl 3-(difluoromethoxy)-4-methylbenzoate (2 g, 9.25 mmol) in CCl₄ (20 mL). The reaction mixture was stirred at 75° C. for 4 h, then at RT overnight. It was then was evaporated to dryness and purified using flash chromatography (SiO₂ column, 0 to 15% EtOAc in hexanes), giving methyl 4-(bromomethyl)-3-(difluoromethoxy)benzoate (1.561 g, 5.29 mmol, 57.2% yield) as an oil.

LC-MS (ES, m/z): [M+H]⁺ 295.0, 297.0.

¹H NMR (400 MHz, CDCl₃) δ 7.88 (dd, J=8.1, 1.5 Hz, 1H), 7.80 (s, 1H), 7.52 (d, J=8.1 Hz, 1H), 6.64 (t, J=73.0 Hz, 1H), 4.57-4.51 (m, 2H), 3.98-3.90 (m, 3H).

Step 3. Cs₂CO₃ (1329 mg, 4.08 mmol) was added to a stirred solution of methyl (3-bromo-7-(butylamino)-1H-pyrazolo[4,3-d]pyrimidin-5-yl)carbamate (700 mg, 2.040 mmol) in DMF (5 mL). After cooling in an ice bath, a solution of methyl 4-(bromomethyl)-3-(difluoro-methoxy)benzoate (572 mg, 1.938 mmol) in DMF (2 mL) was added. The reaction mixture was allowed to warm to RT, stirred for 3 h, diluted with water (20 mL) and was extracted with EtOAc (3×5 mL). The combined organic phases were washed with brine (4×10 mL), dried (MgSO₄), filtered, and concentrated. Flash chromatography (SiO₂ column, loaded in DCM, 0 to 60% EtOAc in hexanes) gave methyl 4-((3-bromo-7-(butylamino)-5-((methoxycarbonyl)amino)-1H-pyrazolo[4,3-d]pyrimidin-1-yl)methyl)-3-(difluoromethoxy)benzoate (275 mg, 0.493 mmol, 24.19% yield) as a solid.

LC-MS (ES, m/z): [M+H]⁺ 557.1, 559.1.

¹H NMR (400 MHz, DMSO-d₆) δ 9.89 (s, 1H), 7.82-7.69 (m, 2H), 7.61-7.14 (m, 2H), 6.87 (d, J=7.9 Hz, 1H), 5.88 (s, 2H), 3.87 (s, 3H), 3.64 (s, 3H), 3.54-3.45 (m, 2H), 1.58-1.46 (m, 2H), 1.19 (dq, J=15.0, 7.4 Hz, 2H), 0.83 (t, J=7.3 Hz, 3H).

Step 4. Methyl 4-((3-bromo-7-(butylamino)-5-((methoxycarbonyl)amino)-1H-pyrazolo[4,3-d]pyrimidin-1-yl)methyl)-3-(difluoromethoxy)benzoate (275 mg, 0.493 mmol) was dissolved in ethanol (15 mL). 10% Pd/C (27 mg) was added. The reaction mixture was evacuated and purged six times, stirred under a hydrogen atmosphere for 2 h, filtered and evaporated to dryness. The residue was dissolved in dioxane (2 mL). NaOH (0.564 mL, 2.82 mmol) was added, and the reaction mixture was stirred at 80° C. for 2 h, then allowed to cool. The reaction mixture was neutralized with 5N HCl and evaporated to dryness. The residue was dissolved in MeOH/water (1:1, 8 mL). The methanol was removed by evaporation. The residual aqueous suspension was filtered. The residue was washed with water, giving 4-((5-amino-7-(butylamino)-1H-pyrazolo[4,3-d]pyrimidin-1-yl)methyl)-3-(difluoromethoxy)benzoic acid (54 mg, 0.133 mmol, 27% yield) as a solid.

LC-MS (ES, m/z): [M+H]⁺=407.22.

¹H NMR (400 MHz, DMSO-d₆) δ 8.50 (br s, 1H), 7.84 (s, 2H), 7.79-7.68 (m, 2H), 7.63-7.05 (t, J=73.2 Hz 1H), 6.97 (d, J=7.9 Hz, 1H), 5.94 (s, 2H), 3.54 (q, J=6.4 Hz, 2H), 1.54 (quin, J=7.2 Hz, 2H), 1.19 (dq, J=14.9, 7.3 Hz, 2H), 0.84 (t, J=7.3 Hz, 3H).

Step 5. A 20 mL scintillation vial was charged with 4-((5-amino-7-(butylamino)-1H-pyrazolo[4,3-d]pyrimidin-1-yl)methyl)-3-(difluoromethoxy)benzoic acid (35 mg, 0.086 mmol), HATU (39.3 mg, 0.103 mmol), (3aR,6aS)-2-methyloctahydropyrrolo[3,4-c]pyrrole (10.87 mg, 0.086 mmol) and DMF (2 mL). DIPEA (0.045 mL, 0.258 mmol) was added. The reaction mixture was stirred at RT for 1 h, filtered, and purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 5% B, 5-45% B over 20 min, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 116, as ditrifluoroacetate salt (12.3 mg, 0.016 mmol, 19% yield).

Compounds 117 and 123 were analogously prepared:

### Example 7—Compound 118

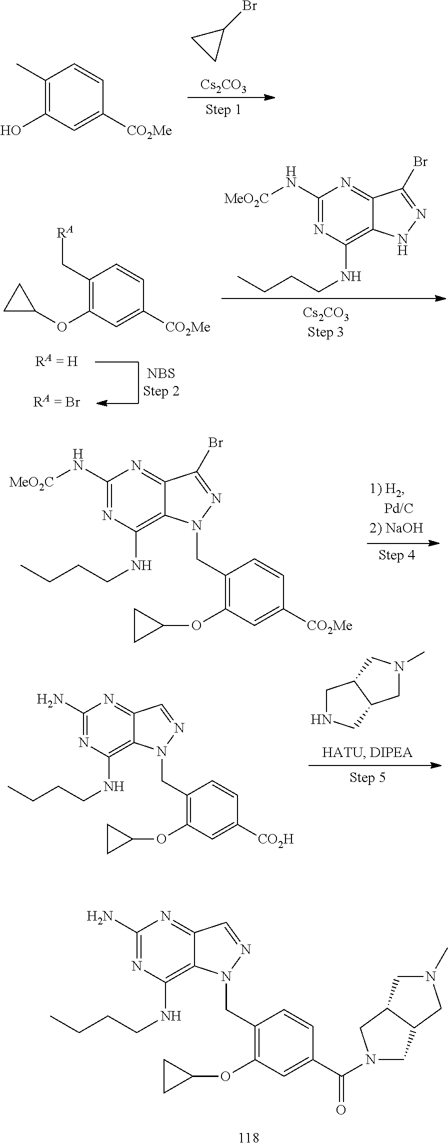

Step 1. A microwave vial was charged with methyl 3-hydroxy-4-methylbenzoate (2 g, 12.04 mmol), bromocyclopropane (1.747 g, 14.44 mmol), Cs₂CO₃ (4.71 g, 14.44 mmol) and DMF (15 mL). The reaction mixture was heated in a microwave oven at 160° C. for 3 h, cooled, poured into water (150 mL), and extracted with EtOAc (3×50 mL). The combined organic phases were washed with brine (4×50 mL), dried (MgSO₄), filtered, and concentrated. Flash chromatography (SiO₂ column, 0 to 5% EtOAc in hexanes) gave methyl 3-cyclopropoxy-4-methylbenzoate (980 mg, 1.901 mmol, 15.79% yield, purity 40%) as an oil, which was used in the next step without further purification.

LC-MS (ES, m/z): [M+H]⁺ 207.1.

Step 2. Methyl 3-cyclopropoxy-4-methylbenzoate (1 g, 1.939 mmol, 40% pure) was dissolved in CCl₄ (5 mL). NBS (0.759 g, 4.27 mmol) and benzoyl peroxide (0.103 g, 0.427 mmol) were added. The reaction mixture was stirred overnight at 70° C., cooled, and evaporated to dryness. Flash chromatography (SiO₂ column, 0 to 10% EtOAc in hexanes) gave methyl 4-(bromomethyl)-3-cyclopropoxybenzoate (550 mg, 1.54 mmol, purity 80%, 80% yield) as a solid. The product was taken on to the next step without further purification.

LC-MS (ES, m/z): [M+H]⁺ 285.0, 287.0.

Step 3. To a stirred solution of methyl (3-bromo-7-(butylamino)-1H-pyrazolo[4,3-d]pyrimidin-5-yl)carbamate (650 mg, 1.894 mmol; US 2020/0038403 A1) in DMF (5 mL) at 0° C. was added Cs₂CO₃ (1296 mg, 3.98 mmol), followed by a solution of methyl 4-(bromomethyl)-3-cyclopropoxybenzoate (540 mg, 1.515 mmol, 80% pure) in DMF (2 mL). The reaction mixture was allowed to warm to RT, stirred overnight, poured into saturated NaHCO₃ solution (100 mL), and extracted with EtOAc (3×50 mL). The combined organic phases were washed with brine (4×50 mL), dried (MgSO₄), filtered and concentrated. Flash chromatography (SiO₂ column, 0 to 70% EtOAc in hexanes) gave methyl 4-((3-bromo-7-(butylamino)-5-((methoxycarbonyl)amino)-1H-pyrazolo[4,3-d]pyrimidin-1-yl)methyl)-3-cyclopropoxybenzoate (153 mg, 0.279 mmol, 14.76% yield) as an oil which solidified on standing.

LC-MS (ES, m/z): [M+H]⁺ 547.2, 549.2.

¹H NMR (400 MHz, DMSO-d₆) δ 9.86 (s, 1H), 7.80 (d, J=1.5 Hz, 1H), 7.53 (dd, J=7.9, 1.5 Hz, 1H), 7.32 (t, J=5.5 Hz, 1H), 6.91 (d, J=7.9 Hz, 1H), 5.72 (s, 2H), 4.03-3.93 (m, 1H), 3.85 (s, 3H), 3.71-3.60 (m, 3H), 3.56-3.45 (m, 2H), 1.56 (quin, J=7.3 Hz, 2H), 1.22 (dq, J=14.8, 7.4 Hz, 2H), 0.85 (t, J=7.4 Hz, 3H), 0.81-0.73 (m, 2H), 0.52-0.41 (m, 2H).

Step 4. Methyl 4-((3-bromo-7-(butylamino)-5-((methoxycarbonyl)amino)-1H-pyrazolo[4,3-d]pyrimidin-1-yl)methyl)-3-cyclopropoxybenzoate (150 mg, 0.274 mmol) was dissolved in EtOH (5 mL) and 10% Pd/C (15 mg) was added. The reaction mixture was evacuated, purged with hydrogen six times, stirred under a hydrogen atmosphere for 1 h, and filtered. Then it was evaporated to dryness. The residue was dissolved in dioxane (3 mL) and NaOH (822 μl, 4.11 mmol) was added. The reaction mixture was stirred at 80° C. for 2 h, cooled, acidified with 5N HCl, and diluted with water (5 mL). The organic volatiles were evaporated off, and the aqueous residue was purified using reverse-phase flash chromatography (C₁₈ column, 0 to 70% acetonitrile in water containing 0.05% TFA) gave 4-((5-amino-7-(butylamino)-1H-pyrazolo[4,3-d]pyrimidin-1-yl)methyl)-3-cyclopropoxybenzoic acid (35 mg, 0.088 mmol, 32% yield) as a solid.

LC-MS (ES, m/z): [M+H]⁺ 397.2.

¹H NMR (400 MHz, DMSO-d₆) δ 8.21 (br t, J=5.6 Hz, 1H), 7.81 (br s, 2H), 7.73 (s, 1H), 7.69 (s, 1H), 7.42 (dd, J=7.9, 1.3 Hz, 1H), 6.81 (d, J=7.9 Hz, 1H), 5.66 (s, 2H), 3.87 (tt, J=5.9, 2.9 Hz, 1H), 3.48 (q, J=6.7 Hz, 2H), 1.48 (quin, J=7.3 Hz, 2H), 1.14 (sxt, J=7.4 Hz, 2H), 0.78 (t, J=7.4 Hz, 3H), 0.75-0.68 (m, 2H), 0.48-0.38 (m, 2H).

Step 5. A 20 mL scintillation vial was charged with 4-((5-amino-7-(butylamino)-1H-pyrazolo[4,3-d]pyrimidin-1-yl)methyl)-3-cyclopropoxybenzoic acid (35 mg, 0.088 mmol), HATU (40.3 mg, 0.106 mmol), (3aR,6aS)-2-methyloctahydropyrrolo[3,4-c]pyrrole (22.28 mg, 0.177 mmol) and DMF (2 mL). DIPEA (0.046 mL, 0.265 mmol) was added. The reaction mixture was stirred at RT for 1 h, filtered, and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 6% B, 6-46% B over 20 min, then a 0-minute hold at 100% B; Flow Rate: 20 mL/min; Column Temperature: 25° C. Fraction collection was triggered by MS signals. Fractions containing the desired product were combined and dried via centrifugal evaporation, giving Compound 118 (17.4 mg, 0.034 mmol, 39% yield).

### Example 8—Compound 124, ditrifluoroacetate

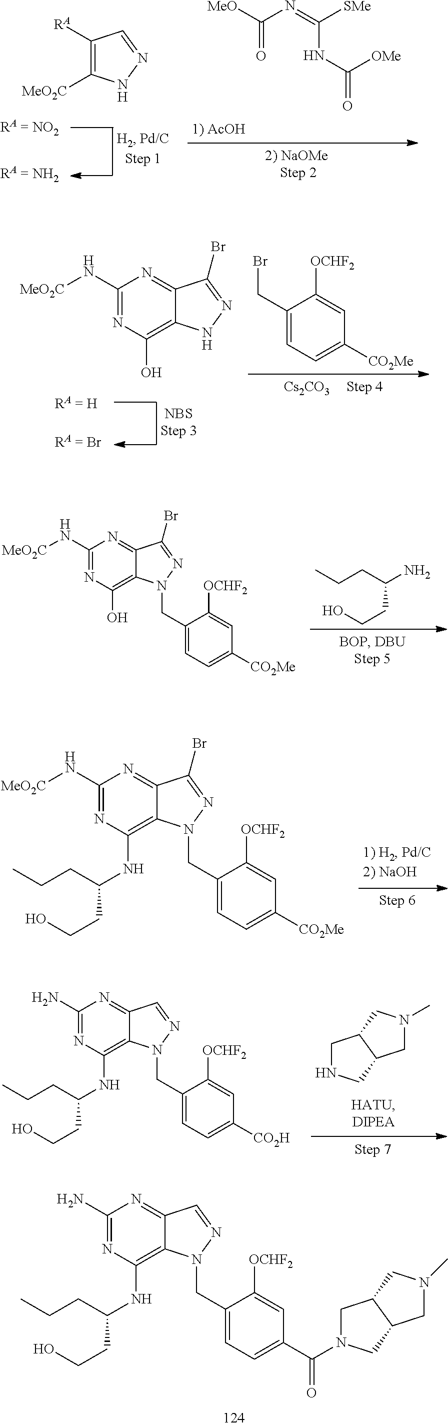

Step 1. 10% Palladium on carbon (0.622 g, 0.584 mmol) was added to a stirred solution of methyl 4-nitro-1H-pyrazole-5-carboxylate (10 g, 58.4 mmol) in EtOH (100 mL). The reaction vessel was evacuated and purged with hydrogen six times. The reaction mixture was stirred under a H₂ (balloon) for 2 days, filtered through CELITE™, and washed with EtOH (100 mL). The filtrate was evaporated to dryness and triturated with ether/hexanes to give methyl 4-amino-1H-pyrazole-5-carboxylate (8.012 g, 56.8 mmol, 97% yield) as a solid.

LC-MS (ES, m/z): [M+H]⁺ 142.1.

Step 2. Methyl 4-amino-1H-pyrazole-5-carboxylate (4 g, 28.3 mmol) was dissolved in MeOH (75 mL). 1,3-Bis(methoxycarbonyl)-2-methyl-2-thiopseudourea (6.43 g, 31.2 mmol) was added, followed by HOAc (6.49 mL, 113 mmol). The reaction mixture was stirred at RT for 5 h. NaOMe (36.7 g, 170 mmol, 25% by weight) was added, followed by more stirring at RT overnight. The reaction mixture was acidified with HOAc. The precipitate was collected by filtration, washed with water (100 mL), THF (100 mL) and ether (100 mL), to give methyl (7-hydroxy-1H-pyrazolo[4,3-d]pyrimidin-5-yl)carbamate (5.098 g, 24.37 mmol, 86% yield) as a solid.

LC-MS (ES, m/z): [M+H]⁺ 210.0.

Step 3. N-bromosuccinimide (4.34 g, 24.38 mmol) was added to a suspension of methyl (7-hydroxy-1H-pyrazolo[4,3-d]pyrimidin-5-yl)carbamate (5.1 g, 24.38 mmol) in DMF (100 mL). The reaction mixture stirred at RT for 1 h, quenched with water (100 mL), stirred for 10 min, filtered, and washed with water (100 mL), THF (2×50 mL) and ether (2×50 mL), giving methyl (3-bromo-7-hydroxy-1H-pyrazolo[4,3-d]pyrimidin-5-yl)carbamate (8.32 g, 23.11 mmol, 95% yield) as a solid.

LC-MS (ES, m/z): [M+H]⁺ 288.0, 290.0.

Step 4. A stirred suspension of methyl (3-bromo-7-hydroxy-1H-pyrazolo[4,3-d]pyrimidin-5-yl)carbamate (2.169 g, 6.78 mmol) and Cs₂CO₃ (2.429 g, 7.46 mmol) in DMF (50 mL) was cooled in an ice bath. A solution of methyl 4-(bromomethyl)-3-(difluoromethoxy)-benzoate (2 g, 6.78 mmol) in DMF (10 mL) was added. The reaction mixture was warmed slowly to RT, stirred overnight, poured into water (400 mL) and saturated NaHCO₃ solution (40 mL), and extracted with EtOAc (3×100 mL). The combined organic phases were washed with brine (4×50 mL), dried (MgSO₄), filtered and concentrated. Trituration using DCM/ether gave methyl 4-((3-bromo-7-hydroxy-5-((methoxycarbonyl)amino)-1H-pyrazolo[4,3-d]pyrimidin-1-yl)methyl)-3-(difluoromethoxy)benzoate (1.937 g, 3.86 mmol, 56.9% yield) as a solid.

LCMS M+H 502.1, 504.1.

¹H NMR (400 MHz, DMSO-d6) δ 11.86-11.58 (m, 1H), 11.58-11.29 (m, 1H), 7.88-7.65 (m, 2H), 7.57-7.07 (m, 2H), 5.94-5.73 (m, 2H), 3.99-3.81 (m, 3H), 3.81-3.67 (m, 3H).

Step 5. A 20 mL scintillation vial was charged with methyl 4-((3-bromo-7-hydroxy-5-((methoxycarbonyl)amino)-1H-pyrazolo[4,3-d]pyrimidin-1-yl)methyl)-3-(difluoromethoxy)-benzoate (1.15 g, 2.290 mmol), (S)-3-aminohexan-1-ol hydrochloride (0.387 g, 2.52 mmol), BOP (1.215 g, 2.75 mmol) and DMSO (10 mL). DBU (1.035 mL, 6.87 mmol) was added. The reaction mixture was stirred at 50° C. overnight, cooled, poured into saturated NaHCO₃ solution (100 mL), and extracted with EtOAc (3×50 mL). The combined organic phases were washed with brine (4×50 mL), dried (MgSO₄), filtered and concentrated. Reverse-phase flash chromatography (100 g C18 column, 0 to 100% acetonitrile in water containing 0.05% TFA) gave methyl (S)-4-((3-bromo-7-((1-hydroxyhexan-3-yl)amino)-5-((methoxycarbonyl)amino)-1H-pyrazolo[4,3-d]pyrimidin-1-yl)methyl)-3-(difluoromethoxy)benzoate (400 mg, 0.532 mmol, 23.24% yield) as a solid.

LC-MS (ES, m/z): [M+H]⁺=601.2, 603.1.

Step 6. 10% Palladium on carbon (40 mg) was added to a stirred suspension of methyl (S)-4-((3-bromo-7-((1-hydroxyhexan-3-yl)amino)-5-((methoxycarbonyl)amino)-1H-pyrazolo[4,3-d]pyrimidin-1-yl)methyl)-3-(difluoromethoxy)benzoate (400 mg, 0.532 mmol) in ethanol (15 mL). The reaction vessel was evacuated and purged with hydrogen six times. The reaction mixture was stirred overnight under a hydrogen atmosphere, filtered, and evaporated to dryness. The residue was dissolved in dioxane (8 mL). NaOH (1.596 mL, 7.98 mmol) was added. After stirring at 80° C. for 2 h and then cooling, the reaction mixture was neutralized using 5N HCl. The dioxane was removed by evaporation. The aqueous residue was purified using reverse-phase flash chromatography (50 g C18 column, 0 to 50% acetonitrile in water containing 10 mM TEAA), giving (S)-4-((5-amino-7-((1-hydroxyhexan-3-yl)amino)-1H-pyrazolo[4,3-d]pyrimidin-1-yl)methyl)-3-(difluoromethoxy)benzoic acid (80 mg, 0.18 mmol, 33% yield) as a solid.

LC-MS (ES, m/z): [M+H]⁺=451.2.

Step 7. A 20 mL scintillation vial was charged with (S)-4-((5-amino-7-((1-hydroxy-hexan-3-yl)amino)-1H-pyrazolo[4,3-d]pyrimidin-1-yl)methyl)-3-(difluoromethoxy)benzoic acid (25 mg, 0.056 mmol), HATU (25.3 mg, 0.067 mmol), (3aR,6aS)-2-methyloctahydropyrrolo[3,4-c]pyrrole (10.51 mg, 0.083 mmol) and DMF (2 mL). DIPEA (0.024 mL, 0.139 mmol) was added. The reaction mixture was stirred at RT for 1 h, filtered, and purified via preparative LC/MS with these conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-min hold at 5% B, 5-45% B over 20 min, then a 0-minute hold at 100% B; Flow Rate: 20 mL/min; Column Temperature: 25° C. Fraction collection was triggered by MS and UV signals; those containing the product were combined and dried via centrifugal evaporation, giving Compound 124, as ditrifluoroacetate salt (24.5 mg, 0.031 mmol, 55.2% yield).

Compound 125 was analogously prepared.

### Example 9—Compound 119

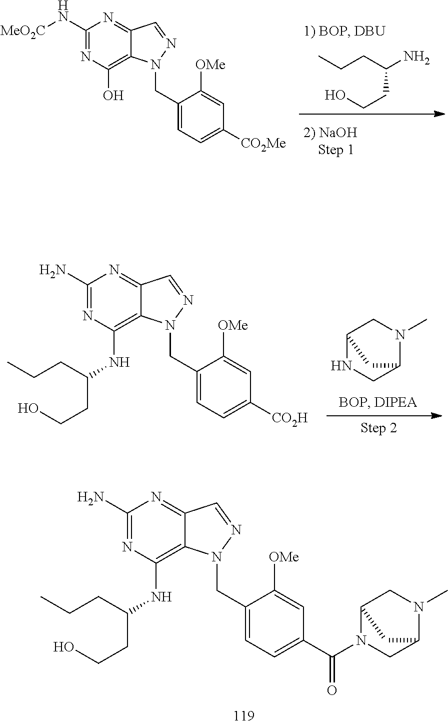

Step 1. DBU (0.856 mL, 5.68 mmol) was added to a suspension of methyl 4-((7-hydroxy-5-((methoxycarbonyl)amino)-1H-pyrazolo[4,3-d]pyrimidin-1-yl)methyl)-3-methoxybenzoate (550 mg, 1.420 mmol; see Step 6 of Example 2 before NaOH treatment) and (S)-3-aminohexan-1-ol hydrochloride 2 (327 mg, 2.130 mmol) in DMSO (5 mL). The reaction mixture was stirred at RT for 10 min, after which it became a clear solution. BOP (1256 mg, 2.84 mmol) was added and followed by stirring at 70° C. for 2 h. The reaction mixture was treated with 5M NaOH (5 mL, 25.00 mmol), stirred at 70° C. for 0.5 h, cooled, and filtered through a syringe filter disc. The filtrate was purified on a preparative reverse C18 column (150 g), eluted with acetonitrile:water (with 0.05% TFA), 0-50% gradient. The desired fraction was frozen and lyophilized to afford (S)-4-((5-amino-7-((1-hydroxyhexan-3-yl)amino)-1H-pyrazolo[4,3-d]pyrimidin-1-yl)methyl)-3-methoxybenzoic acid (860.8 mg, 1.246 mmol, 88% yield).

LCMS ESI: calculated for C₂₀H₂₇N₆O₄=415.2 (M+H+), found 415.2(M+H+).

Step 2. A mixture of (S)-4-((5-amino-7-((1-hydroxyhexan-3-yl)amino)-1H-pyrazolo[4,3-d]pyrimidin-1-yl)methyl)-3-methoxybenzoic acid (50 mg, 0.121 mmol), (1R,4R)-2-methyl-2,5-diazabicyclo[2.2.1]heptane, 2-hydrobromide (66.1 mg, 0.241 mmol) in DMF (1 mL) was treated with Hunig's base (0.105 mL, 0.603 mmol), following by BOP (80 mg, 0.181 mmol). The reaction mixture was stirred at RT for 3 h and filtered through a syringe frit. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 2% B, 2-42% B over 25 min, then a 0-minute hold at 100% B; Flow Rate: 20 mL/min; Column Temperature: 25° C. Fraction collection was triggered by MS signals. Fractions containing the desired product were combined and dried via centrifugal evaporation to yield Compound 119 (6.8 mg, 0.013 mmol, 11.08% yield).

Compound 120 was analogously prepared.

### Example 10—Compound 106

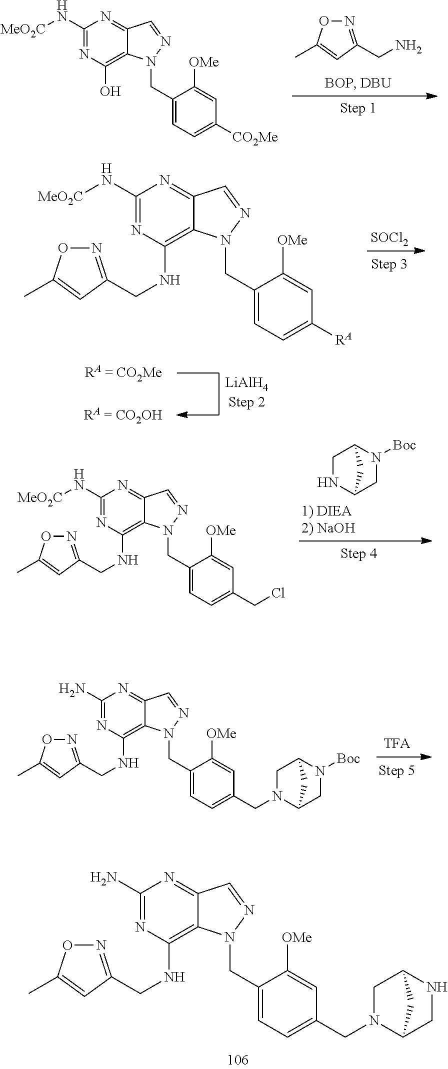

Step 1. A solution of methyl 4-((7-hydroxy-5-((methoxycarbonyl)amino)-1H-pyrazolo[4,3-d]pyrimidin-1-yl)methyl)-3-methoxybenzoate (300 mg, 0.774 mmol; US 2020/0038403 A1) in DMSO (3.9 mL) was treated with (5-methylisoxazol-3-yl)methanamine (174 mg, 1.55 mmol), BOP (411 mg, 0.929 mmol) and DBU (233 μL, 1.55 mmol). The reaction mixture was stirred at RT for 2 h, diluted with EtOAc, and washed with H₂O (3×). The organic layer was dried over Na₂SO₄, filtered and concentrated in vacuo to give methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (353 mg, 95% yield).

¹H NMR (400 MHz, DMSO-d₆) δ 9.80 (s, 1H), 7.99-7.93 (m, 1H), 7.77 (t, J=5.9 Hz, 1H), 7.49 (d, J=1.5 Hz, 1H), 7.45 (dd, J=7.8, 1.5 Hz, 1H), 6.62 (d, J=7.9 Hz, 1H), 6.10 (d, J=0.9 Hz, 1H), 5.80 (s, 2H), 4.73 (d, J=5.9 Hz, 2H), 3.84 (s, 3H), 3.82 (s, 3H), 3.64 (s, 3H), 2.31 (s, 3H).

LC RT: 0.67 min. LC/MS [M+H]⁺=482.3 (Method 1)

Step 2. A solution of methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (190 mg, 0.395 mmol) in THF (10 mL) was cooled to 0° C. and treated with LiAlH₄ (1M in THF, 691 μL, 0.691 mmol). The reaction mixture was stirred for 15 min at 0° C., quenched with MeOH and Rochelle's salt (saturated aqueous solution), and stirred at RT for 1 h. The mixture was extracted with EtOAc (3×). The combined organic layers were washed with H₂O, dried over Na₂SO₄, filtered and concentrated in vacuo to give methyl (1-(4-(hydroxymethyl)-2-methoxybenzyl)-7-(((5-methylisoxazol-3-yl)methyl)amino)-1H-pyrazolo[4,3-d]pyrimidin-5-yl)carbamate (160 mg, 89% yield).

¹H NMR (400 MHz, DMSO-d₆) δ 9.77-9.75 (m, 1H), 7.90-7.88 (m, 1H), 7.72 (br t, J=5.7 Hz, 1H), 6.94 (s, 1H), 6.76 (d, J=7.5 Hz, 1H), 6.61-6.57 (m, 1H), 6.15 (d, J=0.8 Hz, 1H), 5.68 (s, 2H), 5.16 (t, J=5.7 Hz, 1H), 4.73 (br d, J=5.8 Hz, 2H), 4.44 (d, J=5.6 Hz, 2H), 3.70 (s, 3H), 3.62 (s, 3H), 2.33 (s, 3H).

LC RT: 0.58 min. LCMS [M+H]⁺=454.3 (Method 1)

Step 3. A solution of methyl (1-(4-(hydroxymethyl)-2-methoxybenzyl)-7-(((5-methyl-isoxazol-3-yl)methyl)amino)-1H-pyrazolo[4,3-d]pyrimidin-5-yl)carbamate (159 mg, 0.350 mmol) in DCM (3.5 mL) was treated with SOCl₂ (128 μL, 1.76 mmol). The reaction mixture was stirred at RT for 15 min and concentrated in vacuo. The residue was re-dissolved in DCM and concentrated in vacuo to give methyl (1-(4-(chloromethyl)-2-methoxybenzyl)-7-(((5-methylisoxazol-3-yl)methyl)amino)-1H-pyrazolo[4,3-d]pyrimidin-5-yl)carbamate (182 mg, 100%).

LC RT: 0.80 min. LCMS [M+H]⁺=472.3 (Method 1)

Step 4. A solution of methyl (1-(4-(chloromethyl)-2-methoxybenzyl)-7-(((5-methylisoxazol-3-yl)methyl)amino)-1H-pyrazolo[4,3-d]pyrimidin-5-yl)carbamate (25 mg, 0.053 mmol in DMF (1.1 mL) was treated with tert-butyl (1S,4S)-2,5-diazabicyclo[2.2.1]heptane-2-carboxylate (31.5 mg, 0.159 mmol). The reaction mixture was stirred at 70° C. for 2 h and concentrated in vacuo. The residue was re-dissolved in dioxane (0.5 mL) at RT, treated with NaOH (10M aqueous solution, 26 μL, 0.26 mmol) and heated to 70° C.; at 3 h and 6 h additional NaOH (10M aqueous Solution, 100 μL, 1 mmol) was added to the reaction mixture. After 10 h, the reaction mixture was cooled to RT, neutralized with HOAc and concentrated in vacuo. The crude product was dissolved in DMF, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 20% B, 20-60% B over 23 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give tert-butyl (1S,4S)-5-({4-[(5-amino-7-{[(5-methyl-1,2-oxazol-3-yl)methyl]amino}-1H-pyrazolo[4,3-d]pyrimidin-1-yl)methyl]-3-methoxy-phenyl}methyl)-2,5-diazabicyclo[2.2.1]heptane-2-carboxylate (8.1 mg, 27%).

¹H NMR (500 MHz, DMSO-d₆) δ 7.59 (s, 1H), 7.27 (br d, J=2.5 Hz, 1H), 6.96 (s, 1H), 6.78 (br d, J=7.4 Hz, 1H), 6.50 (d, J=7.7 Hz, 1H), 5.99 (s, 1H), 5.75 (s, 2H), 5.60 (s, 2H), 4.64 (br s, 2H), 4.15 (br d, J=17.3 Hz, 1H), 3.74 (s, 3H), 3.40 (br s, 1H), 3.35 (br d, J=9.6 Hz, 1H), 3.07 (br dd, J=19.1, 9.5 Hz, 1H), 2.77-2.71 (m, 1H), 2.47-2.39 (m, 1H), 2.33 (s, 3H), 1.77 (br d, J=8.5 Hz, 1H), 1.65-1.56 (m, 1H), 1.38 (br s, 9H)

LC RT: 1.12 min. LCMS [M+H]⁺=576.2 (Method 3).

Step 5. The tert-butyl (1S,4S)-5-({4-[(5-amino-7-{[(5-methyl-1,2-oxazol-3-yl)methyl]amino}-1H-pyrazolo[4,3-d]pyrimidin-1-yl)methyl]-3-methoxyphenyl}methyl)-2,5-diazabicyclo[2.2.1]heptane-2-carboxylate was treated with DCM (1.0 mL) and TFA (0.5 mL, 6 mmol), stirred at 50° C. for 30 min and concentrated. The crude product was dissolved in DMF, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 5% B, 5-45% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 106 (10.0 mg, 39%).

The following compounds were analogously prepared: Compound 126, Compound 127, and Compound 128

### Example 11—Compound 129

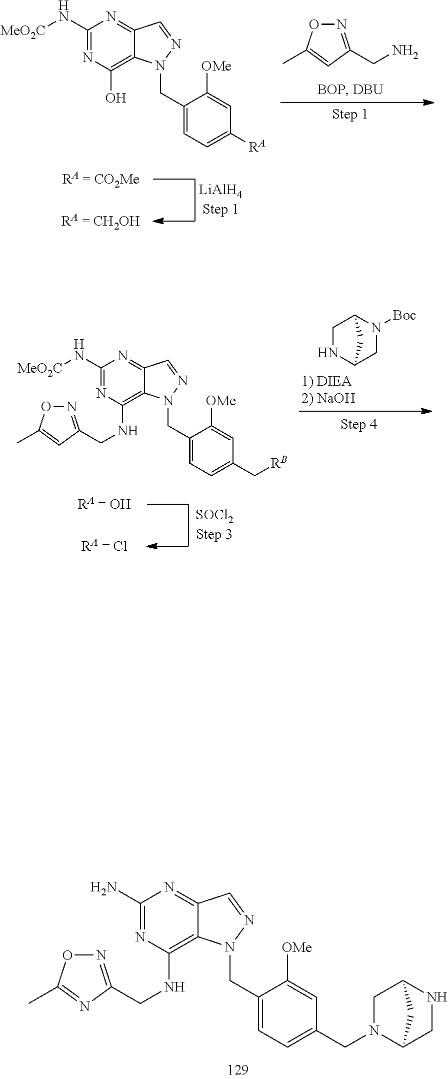

Step 1. A solution of methyl 4-((5-((tert-butoxycarbonyl)amino)-7-hydroxy-1H-pyrazolo[4,3-d]pyrimidin-1-yl)methyl)-3-methoxybenzoate (685 mg, 1.59 mmol; US 2020/0038403 A1) in THF (16 mL) was cooled to 0° C. and treated with LiAlH₄ (1 M in THF, 2.8 mL, 2.8 mmol). The reaction mixture was stirred for 15 min at 0° C., quenched with H₂O and Rochelle's salt (saturated aqueous solution), and stirred at RT for 3 h. The organic layer was absorbed onto CELITE™ and purified via column chromatography (24 g SiO₂; 0 to 20% MeOH-DCM gradient elution) to give tert-butyl (7-hydroxy-1-(4-(hydroxymethyl)-2-methoxybenzyl)-1H-pyrazolo[4,3-d]pyrimidin-5-yl)carbamate (460 mg, 72% yield).

¹H (400 MHz, DMSO-d₆) δ 11.69-11.43 (m, 1H), 10.95-10.62 (m, 1H), 7.87-7.79 (m, 1H), 6.97 (s, 1H), 6.77 (d, J=7.7 Hz, 1H), 6.59 (d, J=7.8 Hz, 1H), 5.66 (s, 2H), 5.16 (t, J=5.8 Hz, 1H), 4.45 (d, J=5.8 Hz, 2H), 3.79 (s, 3H), 1.49 (s, 9H). LC RT: 0.77 min.

LCMS [M+H]⁺=402.2 (Method 1)

Step 2. A solution of tert-butyl (7-hydroxy-1-(4-(hydroxymethyl)-2-methoxybenzyl)-1H-pyrazolo[4,3-d]pyrimidin-5-yl)carbamate (460 mg, 1.15 mmol) in DMSO (5.7 mL) was treated with (5-methyl-1,2,4-oxadiazol-3-yl)methanamine-HCl (223 mg, 1.49 mmol), BOP (760 mg, 1.72 mmol) and DBU (0.69 mL, 4.6 mmol). The reaction mixture was stirred at RT for 2 h, diluted with EtOAc, and washed with H₂O (2×). The organic layer was absorbed onto CELITE™ and purified via column chromatography (100 g C18 gold column; Mobile Phase A: 5:95 acetonitrile:water with 0.05% TFA; Mobile Phase B: 95:5 acetonitrile:water with 0.05% TFA; Flow Rate: 60 mL/min, 30-50% gradient). The purified product was dissolved in DCM and washed with saturated aqueous NaHCO₃ solution. The organic layer was dried over Na₂SO₄, filtered and concentrated in vacuo to give tert-butyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (190 mg, 33% yield).

¹H NMR (400 MHz, DMSO-d₆) δ 9.24-9.15 (m, 1H), 7.87 (s, 1H), 7.72 (t, J=5.8 Hz, 1H), 6.95 (s, 1H), 6.82-6.75 (m, 1H), 6.73-6.68 (m, 1H), 5.68 (s, 2H), 5.17 (t, J=5.7 Hz, 1H), 4.87 (d, J=5.7 Hz, 2H), 4.44 (d, J=5.7 Hz, 2H), 3.76 (s, 3H), 2.55 (s, 3H), 1.43 (s, 9H).

LC RT: 0.75 min. LC/MS [M+H]⁺=497.2 (Method 1)

Step 3. A solution of tert-butyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (161 mg, 0.320 mmol) in DCM (0.65 mL) was treated with SOCl₂ (71 μL, 0.97 mmol). The reaction mixture was stirred at RT for 15 min and concentrated in vacuo. The residue was re-dissolved in DCM and concentrated in vacuo to give tert-butyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (166 mg, 100%).

LC RT: 0.89 min. LCMS [M+H]⁺=515.2 (Method 1)

Step 4. A solution of tert-butyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (33 mg, 0.064 mmol in DMF (1.3 mL) was treated with tert-butyl (1S,4S)-2,5-diazabicyclo[2.2.1]heptane-2-carboxylate (38.3 mg, 0.193 mmol). The reaction mixture was stirred at 70° C. for 2 h and concentrated in vacuo. The residue was dissolved in dioxane (1.3 mL) and treated with HCl (4 M in dioxane, 0.40 mL, 1.6 mmol), stirred at 40° C. for 30 min, and concentrated. The crude product was dissolved in DMF, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 0% B, 0-48% B over 23 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 129 (16.2 mg, 53% yield).

Compound 130 was analogously prepared.

### Example 12—Compound 128

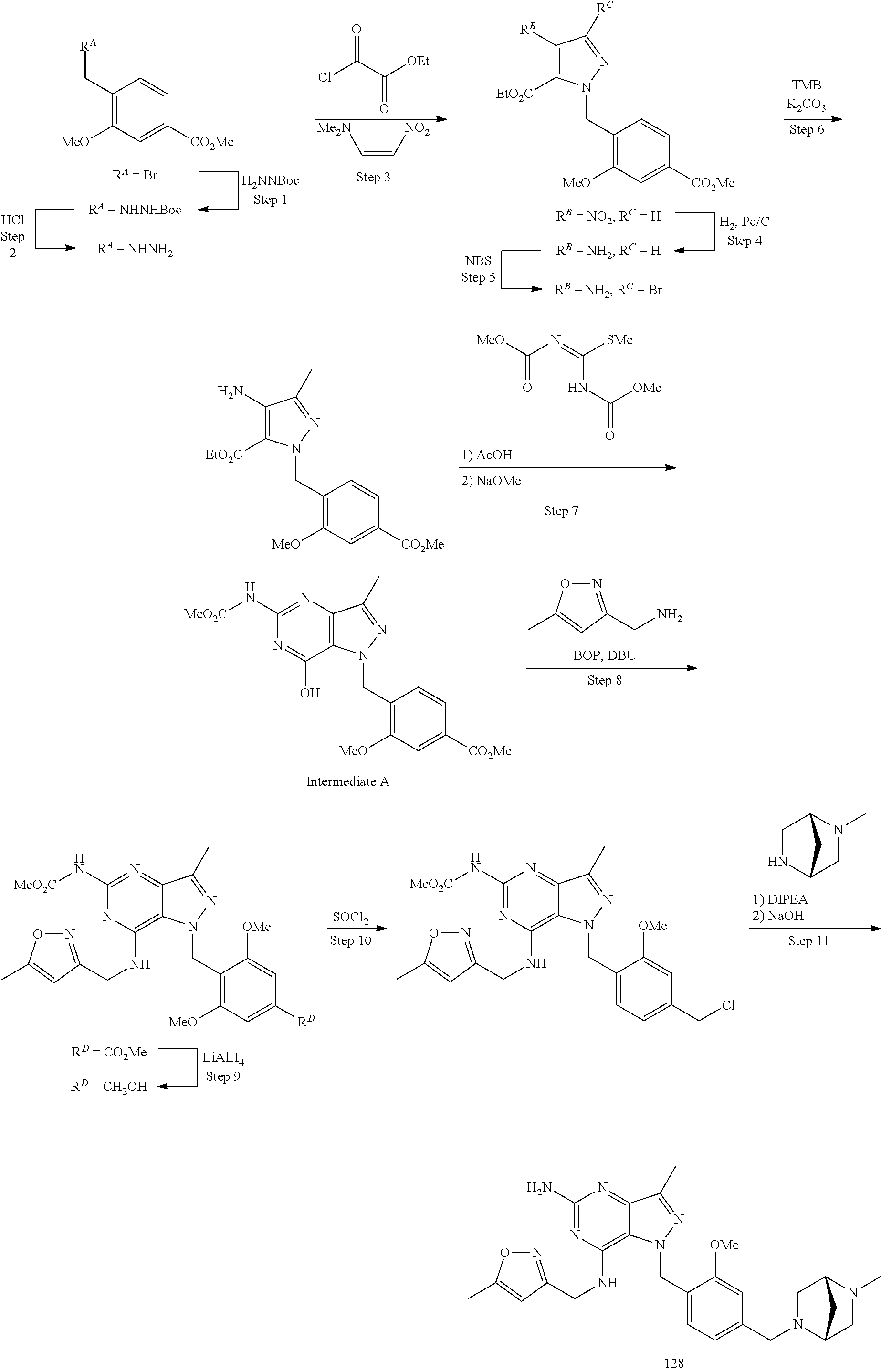

Step 1: A solution of tert-butyl hydrazinecarboxylate (12.75 g, 96 mmol) and DIPEA in DMF (24 mL) at RT was treated with the dropwise addition of methyl 4-(bromomethyl)-3-methoxybenzoate (5 g, 19.30 mmol) in 24 mL of DMF via additional funnel over 1 hour. The reaction mixture was stirred at RT overnight. EtOAc (135 mL) and H₂O (75 mL) were added and the biphasic mixture was stirred for 30 minutes. The reaction mixture was poured into a separatory funnel and the aqueous layer was removed. The organic layer was washed with 2 additional portions of H₂O (75 mL), 2 portions of 10% LiCl solution (75 mL), dried over Na₂SO₄ and concentrated. Column chromatography (Isco, 220 g SiO₂, 0% CH₂Cl₂ (5 min) then 15% EtOAc-CH₂Cl₂) provided tert-butyl 2-(2-methoxy-4-(methoxycarbonyl)benzyl)hydrazine-1-carboxylate as clear oil (3.85 g).

¹H NMR (400 MHz, CHLOROFORM-d) δ 7.64 (dd, J=7.7, 1.5 Hz, 1H), 7.56 (d, J=1.5 Hz, 1H), 7.37 (d, J=7.7 Hz, 1H), 6.08-5.87 (m, 1H), 4.07 (s, 2H), 3.94 (d, J=4.6 Hz, 6H), 1.50-1.40 (m, 9H).

LC/MS [M+H]⁺ 311.2; LC RT=0.80 min (Method 1).

Step 2: tert-Butyl 2-(2-methoxy-4-(methoxycarbonyl)benzyl)hydrazine-1-carboxylate (25.4 g, 82 mmol) was dissolved in MeOH (164 mL) at RT. 4 N HCl-dioxane (123 ml, 59.5 mmol) was added and the reaction was stirred at RT overnight. The white precipitate was collected by filtration and dried to afford methyl 4-(hydrazineylmethyl)-3-methoxybenzoate, 2-HCl (20 g). 1H NMR (400 MHz, DMSO-d6) δ 9.12 (br s), 7.62-7.55 (m, 1H), 7.53-7.47 (m, 2H), 4.10 (s, 2H), 3.88 (s, 3H), 3.87 (s, 3H)

LC/MS [M+H]⁺ 211.1; LC RT=0.51 min. (Method 1)

Step 3: A solution of (E)-N,N-dimethyl-2-nitroethen-1-amine (46.4 g, 400 mmol) and pyridine (420 ml, 5195 mmol) in CH₂Cl₂ (799 ml) was cooled to −10° C. and slowly treated with ethyl 2-chloro-2-oxoacetate (51.4 ml, 460 mmol). The reaction mixture was allow to warm to 25° C. over 2 h and stirred overnight. The CH₂Cl₂ was removed by rotary evaporation and methyl 4-(hydrazineylmethyl)-3-methoxybenzoate dihydrochloride (31.7 g, 112 mmol) was added to the reaction mixture in one portion. The solution was stirred for 2 h at RT and the solvent was removed under vacuum. The residue was washed with water, 1N aqueous HCl solution and extracted with EtOAc (3×). The organic layers were dried over Na₂SO₄, and concentrated. The residue was dissolved in CH₂Cl₂, passed through a short silica gel column and recrystallized from ethanol to afford ethyl 1-(2-methoxy-4-(methoxycarbonyl)benzyl)-4-nitro-1H-pyrazole-5-carboxylate (29.4 g).

¹H NMR (400 MHz, CHLOROFORM-d) δ 8.06 (s, 1H), 7.64 (dd, J=7.9, 1.5 Hz, 1H), 7.56 (d, J=1.5 Hz, 1H), 7.13 (d, J=7.8 Hz, 1H), 5.53 (s, 2H), 4.45 (q, J=7.2 Hz, 2H), 3.94 (s, 3H), 3.88 (s, 3H), 1.37 (t, J=7.2 Hz, 3H).

LC/MS [M+Na]⁺386.0; LC RT=0.98 min (Method 1).

Step 4: Ethyl 4-amino-1-(2-methoxy-4-(methoxycarbonyl)benzyl)-1H-pyrazole-5-carboxylate (3.04 g, 9.12 mmol, 86% yield) and Pd-C(1.131 g, 0.531 mmol) were suspended in EtOAc/MeOH (1:1) (152 mL). The reaction flask was evacuated under vacuum and purged with H₂ (3×) before stirring under balloon pressure of H₂ (g). After 5 h, the reaction mixture filtered through CELITE™, and fresh Pd-C(1.131 g, 0.531 mmol) was added. The reaction flask was evacuated under vacuum and purged with H₂ (3×) before stirring for 16 h under balloon pressure of H₂. The reaction mixture was filtered through CELITE™, concentrated and dried under vacuum to afford ethyl 4-amino-1-(2-methoxy-4-(methoxycarbonyl)benzyl)-1H-pyrazole-5-carboxylate (3.04 g) as a cream powder.

¹H NMR (400 MHz, DMSO-d6) δ 7.52-7.49 (m, 1H), 7.47 (dd, J=7.9, 1.5 Hz, 1H), 7.19 (s, 1H), 6.40 (d, J=7.8 Hz, 1H), 5.54 (s, 2H), 5.10 (s, 1H), 4.15 (q, J=7.1 Hz, 2H), 3.91 (s, 3H), 3.84 (s, 3H), 1.14 (t, J=7.1 Hz, 3H).

LC/MS [M+H]⁺ 334.1; LC/RT=0.85 min. (Method 2).

Step 5: Ethyl 4-amino-1-(2-methoxy-4-(methoxycarbonyl)benzyl)-1H-pyrazole-5-carboxylate (1.65 g, 4.95 mmol) was dissolved in CHCl₃ (49.5 ml) and cooled to 0° C. NBS (0.925 g, 5.20 mmol) was added to the mixture in one portion. After 15 minutes, the reaction was diluted with CHCl₃ and vigorously stirred with 10% aqueous sodium thiosulfate solution for 10 minutes. The organic phase was separated, washed with H₂O, dried over MgSO₄ and concentrated. The crude product was purified by column chromatography (80 g SiO₂, 0 to 50% EtOAc-hexane gradient elution) to afford ethyl 4-amino-3-bromo-1-(2-methoxy-4-(methoxycarbonyl)benzyl)-1H-pyrazole-5-carboxylate (1.32 g) as a white solid.

¹H NMR (400 MHz, DMSO-d6) δ 7.61-7.41 (m, 2H), 6.55 (d, J=8.3 Hz, 1H), 5.56 (s, 2H), 5.02 (s, 2H), 4.20 (q, J=7.1 Hz, 2H), 3.90 (s, 3H), 3.85 (s, 3H), 1.15 (t, J=7.1 Hz, 3H).

LC/MS [M+H]⁺ 412.2; LC RT=1.02 min (Method 1).

Step 6: Ethyl 4-amino-3-bromo-1-(2-methoxy-4-(methoxycarbonyl)benzyl)-1H-pyrazole-5-carboxylate (741.2 mg, 67.1% yield), K₂CO₃ (1.098 g, 7.94 mmol) and 2,4,6-trimethyl-1,3,5,2,4,6-trioxatriborinane (3.5 M in THF) (1.816 ml, 6.36 mmol) were suspended in dioxane (26.5 ml):Water (5.30 ml) (5:1). A stream of N₂ was bubbled through the reaction mixture for 5 min before the addition of PdCl₂(dppf)-CH₂Cl₂ adduct (0.052 g, 0.064 mmol) and continued for another 4 min before sealing the reaction vessel and heating to 90° C. After 3 h, additional portions of 2,4,6-trimethyl-1,3,5,2,4,6-trioxatriborinane (3.5 M in THF) (0.908 ml, 3.18 mmol) and PdCl₂(dppf)-CH₂Cl₂ adduct (0.052 g, 0.064 mmol) were added. The reaction mixture was stirred at 100° C. for 16 h, cooled, diluted with 100 mL of EtOAc, and filtered through CELITE™, washing with additional EtOAc. The crude product was concentrated onto 4 g CELITE™. Column chromatography (80 g SiO₂, 0 to 30% EtOAc-CH₂Cl₂ gradient elution) afforded the expected product, ethyl 4-amino-1-(2-methoxy-4-(methoxycarbonyl)benzyl)-3-methyl-1H-pyrazole-5-carboxylate (741 mg) as a cream solid.

¹H NMR (400 MHz, DMSO-d6) δ 7.49 (d, J=1.5 Hz, 1H), 7.46 (dd, J=7.9, 1.5 Hz, 1H), 6.40 (d, J=7.8 Hz, 1H), 5.48 (s, 2H), 4.94-4.86 (m, 2H), 4.14 (q, J=7.0 Hz, 2H), 3.90 (s, 3H), 3.84 (s, 3H), 2.10 (s, 3H), 1.15-1.08 (m, 3H).

LC/MS [M+H]⁺ 348.2; LC/RT=0.89 min. (Method 1).

Step 7: Ethyl 4-amino-1-(2-methoxy-4-(methoxycarbonyl)benzyl)-3-methyl-1H-pyrazole-5-carboxylate (742 mg, 2.136 mmol) was suspended in MeOH (10.800 mL) and heated gently with vigorous stirring to solubilize the material. 1,3-bis-(Methoxycarbonyl)-2-methyl-2-thiopseudourea (661 mg, 3.20 mmol), was added followed by AcOH (0.611 mL, 10.68 mmol). The reaction mixture was stirred at RT for 16 h. An additional portion of AcOH was added (0.049 mL, 0.854 mmol) and the reaction mixture was stirred at RT for another 72 h before the addition of NaOMe (25% wt in MeOH) (5.69 mL, 25.6 mmol). After stirring for 3 h, the reaction mixture was re-acidified with AcOH. The product was collected by filtration, air-dried for 10 minutes and thoroughly dried in a chem-dry oven to afford methyl 4-((7-hydroxy-5-((methoxy-carbonyl)amino)-3-methyl-1H-pyrazolo[4,3-d]pyrimidin-1-yl)methyl)-3-methoxybenzoate (Intermediate A) (722.0 mg) as a cream-colored solid.

¹H NMR (400 MHz, DMSO-d6) δ 11.58-11.17 (m, 2H), 7.51 (d, J=1.4 Hz, 1H), 7.49-7.42 (m, 1H), 6.67 (d, J=7.9 Hz, 1H), 5.67 (s, 2H), 3.90 (s, 3H), 3.84 (s, 3H), 3.71 (s, 3H), 2.31 (s, 3H).

LC/MS [M+H]⁺ 402.3; LC RT=0.86 min (Method 1).

Step 8. A suspension of methyl 4-((7-hydroxy-5-((methoxycarbonyl)amino)-3-methyl-1H-pyrazolo[4,3-d]pyrimidin-1-yl)methyl)-3-methoxybenzoate (Intermediate A, 200 mg, 0.498 mmol) and BOP (331 mg, 0.747 mmol) in DMF (2491 μL) at RR was treated with (5-methylisoxazol-3-yl)methanamine (72.6 mg, 0.648 mmol) and DBU (3 eq) (225 μl, 1.495 mmol). The reaction mixture was heated to 40° C. After 15 minutes, an additional portion of DBU (2 eq) (150 μL, 0.997 mmol) was added. The reaction was stirred at 40° C. for 16 h, cooled, and partitioned between EtOAc and half-saturated aqueous NaHCO₃ solution. The organic phase was separated and the aqueous phase was extracted with EtOAc (2×). The combined organic layers were washed sequentially with 10% aqueous LiCl solution and brine, dried over Na₂SO₄ and concentrated. Column chromatography (12 g SiO₂, 0 to 10% CH₃OH—CH₂Cl₂ gradient elution) afforded methyl 3-methoxy-4-((5-((methoxycarbonyl)amino)-3-methyl-7-(((5-methylisoxazol-3-yl)methyl)amino)-1H-pyrazolo[4,3-d]pyrimidin-1-yl)methyl)benzoate (201.1 mg).

LC/MS [M+H]⁺ 496.2; LC RT=0.79 min (Method 1).

Step 9. Methyl 3-methoxy-4-((5-((methoxycarbonyl)amino)-3-methyl-7-(((5-methylisoxazol-3-yl)methyl)amino)-1H-pyrazolo[4,3-d]pyrimidin-1-yl)methyl)benzoate (200 mg, 0.404 mmol) was suspended in THF at RT and sonicated to aid dissolution. LiAlH₄ (1M in THF) (807 μl, 0.807 mmol) was added dropwise over 10 min. After 20 min, the reaction was quenched with MeOH and partitioned between EtOAc and Rochelle's salts. The biphasic mixture was stirred at RT for 2 h. The aqueous layer was separated and re-extracted with EtOAc (1×). The combined organic layers were washed with brine and concentrated. Column chromatography (12 g SiO₂, 0 to 10% CH₃OH—CH₂Cl₂ gradient elution) afforded methyl (1-(4-(hydroxymethyl)-2-methoxybenzyl)-3-methyl-7-(((5-methylisoxazol-3-yl)methyl)amino)-1H-pyrazolo[4,3-d]pyrimidin-5-yl)carbamate (73 mg).

LC/MS [M+H]⁺ 468.4; LC RT=0.62 min. (Method 1).

Step 10. Methyl (1-(4-(hydroxymethyl)-2-methoxybenzyl)-3-methyl-7-(((5-methylisoxazol-3-yl)methyl)amino)-1H-pyrazolo[4,3-d]pyrimidin-5-yl)carbamate (73 mg, 0.156 mmol) was dissolved in CH₂Cl₂ (1562 μL) at RT. SOCl₂ (57.0 μl, 0.781 mmol) was added and the reaction mixture was stirred for 20 min. Concentration afforded methyl (1-(4-(chloromethyl)-2-methoxybenzyl)-3-methyl-7-(((5-methylisoxazol-3-yl)methyl)amino)-1H-pyrazolo[4,3-d]pyrimidin-5-yl)carbamate (80 mg) in sufficient purity to use without further purification.

LC/MS [M+H]⁺ 486.1; LC RT=0.83 min (Method 1).

Step 11. A stock solution of methyl (1-(4-(chloromethyl)-2-methoxybenzyl)-3-methyl-7-(((5-methylisoxazol-3-yl)methyl)amino)-1H-pyrazolo[4,3-d]pyrimidin-5-yl)carbamate (20 mg, 0.041 mmol) in acetonitrile (412 μL) was added to (1R,4R)-2-methyl-2,5-diazabicyclo[2.2.1]heptane, 2-hydrobromide (33.8 mg, 0.123 mmol) in a 2-dram vial. DIPEA (21.57 μl, 0.123 mmol) was added. The reaction mixture was heated to 70° C., cooled, and concentrated. The residue was re-dissolved in dioxane (400 μL) and treated with 10 M NaOH solution (82 μL, 0.823 mmol). The reaction mixture was heated to 80° C. for 5 h, cooled, neutralized with AcOH (42 μL), and concentrated. The crude product was dissolved in DMF-H₂O, filtered through a PTFE frit and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to afford Compound 128 (8.6 mg).

Intermediate A in the above scheme can also be used to make other compounds according to this disclosure, mutatis mutandis.

### Example 13—Compound 131

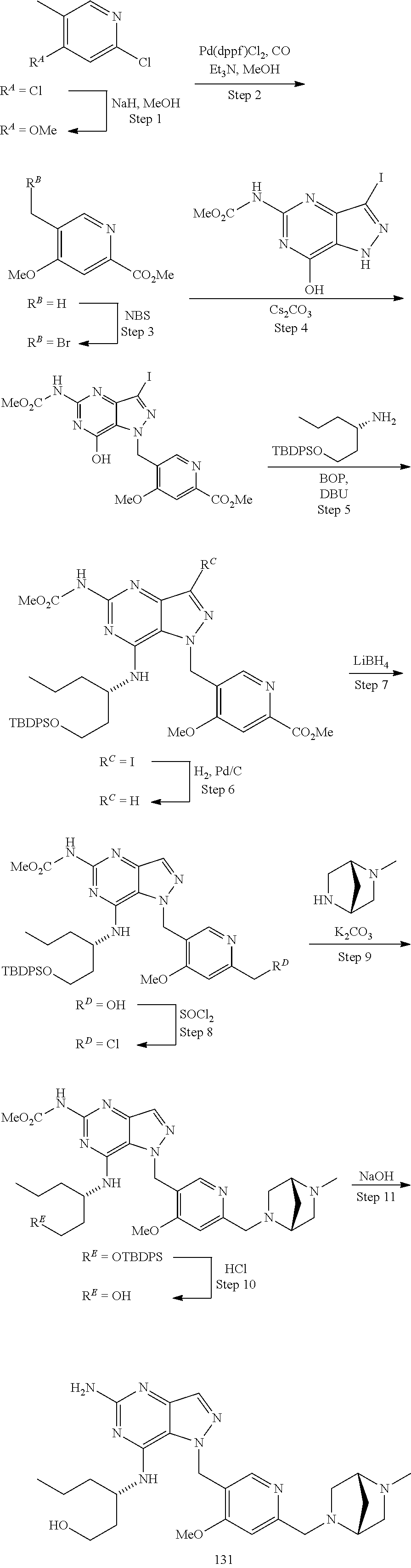

Step 1. To a suspension of sodium hydride (5.92 g, 148 mmol) in diethyl ether (25 mL) and DMF (25 mL) was added methanol (6.49 mL, 160 mmol) at 0° C. under an inert atmosphere in a 50-mL two-necked flask. After 20 min., a solution of 2,4-dichloro-5-methylpyridine (commercially available, 20 g, 123 mmol) in diethyl ether (25 mL) was added dropwise, and then the mixture was allowed to warm to RT. After 12 h, crushed ice was added to the reaction mixture, which was then extracted by DCM (2×250 mL). The combined organic layers were dried over Na₂SO₄ and evaporated to get the crude product which was purified by column chromatography using 80 g silica gel column and 5-30% ethyl acetate in petroleum ether as eluent to get 2-chloro-4-methoxy-5-methylpyridine (18 g, 93%).

LC-MS m/z 158 [M+H]⁺.

Step 2. A solution of 2-chloro-4-methoxy-5-methylpyridine (19 g, 121 mmol) in MeOH (350 mL) was added into a reactor followed by DMF (350 mL). The mixture was purged with nitrogen gas, after which Pd(dppf)Cl₂-DCM (19.69 g, 24.11 mmol) and triethylamine (50.3 mL, 362 mmol) were added. After purging with nitrogen gas, the reaction mixture was stirred for 18 h under 10 bar pressure of carbon monoxide at 100° C. The reaction mixture was collected from the reactor and evaporated to get the crude product which was purified by column chromatography using 80 g silica gel column and 5-30% ethyl acetate in petroleum ether as eluent to get methyl 4-methoxy-5-methylpicolinate (18.4 g, 84%).

LC-MS m/z 182 [M+H]⁺.

Step 3. To a mixture of methyl 4-methoxy-5-methylpicolinate (5.8 g, 32.0 mmol) in CCl₄ (50 mL) was added NBS (5.70 g, 32.0 mmol) and AIBN (1.051 g, 6.40 mmol), and the reaction mixture was heated at 60° C. for 12 h under inert atmosphere. Additional NBS (0.5 equiv.) and AIBN (0.1 equiv.) were added and the reaction was stirred for 18 h. Saturated aqueous sodium bicarbonate was added to the reaction mixture, which was then extracted by DCM (2×150 mL). The combined organic layers were dried over Na₂SO₄ and evaporated to get the crude product which was purified by column chromatography using 80 g silica gel column and 20-50% ethyl acetate in petroleum ether as eluent to get methyl 5-(bromomethyl)-4-methoxypicolinate (5.5 g, 66%).

LC-MS m/z 260/262 [M+H]⁺.

Step 4. A mixture of methyl (7-hydroxy-3-iodo-1H-pyrazolo[4,3-d]pyrimidin-5-yl)carbamate (6 g, 17.91 mmol) in DMF (10 mL) was cooled to 0° C. and methyl 5-(bromo-methyl)-4-methoxypicolinate (4.66 g, 17.91 mmol) was added, followed by Cs₂CO₃ (11.67 g, 35.8 mmol). The reaction mixture was stirred for 1 h at the same temperature. The reaction mixture was stirred at RT for 2 h. Crushed ice was added to the reaction mixture to get a pale yellow precipitate which was filtered off through a sintered funnel and washed with 30% ethyl acetate in petroleum ether. The solid was dried under vacuum to get the crude product which was purified by column chromatography using 80 g silica gel column and 2-10% methanol in DCM as eluent to get methyl 5-((7-hydroxy-3-iodo-5-((methoxycarbonyl)amino)-1H-pyrazolo[4,3-d]pyrimidin-1-yl)methyl)-4-methoxypicolinate (4 g, 43%).

LC-MS m/z 515 [M+H]⁺.

Step 5. To a stirred solution of methyl 5-((7-hydroxy-3-iodo-5-((methoxycarbonyl)-amino)-1H-pyrazolo[4,3-d]pyrimidin-1-yl)methyl)-4-methoxypicolinate (500 mg, 0.972 mmol) in DMSO (5 mL) was added successively (S)-1-((tert-butyldiphenylsilyl)oxy)hexan-3-amine (415 mg, 1.167 mmol) (US 2020/0038403 A1, FIG. 8, compound 71a), BOP (645 mg, 1.458 mmol), and DBU (0.440 mL, 2.92 mmol), and the reaction mixture was stirred at 45° C. for 4 h. Crushed ice was added to the reaction mixture, which was then extracted by DCM (2×150 mL). The combined organic layers were dried over Na₂SO₄ and evaporated to get the crude product which was purified by column chromatography using 80 g silica gel column and 15-40% ethyl acetate in petroleum ether as eluent to get methyl (S)-5-((7-((1-((tert-butyldiphenylsilyl)oxy)-hexan-3-yl)amino)-3-iodo-5-((methoxycarbonyl)amino)-1H-pyrazolo[4,3-d]pyrimidin-1-yl)methyl)-4-methoxypicolinate (400 mg, 48%).

LC-MS m/z 852 [M+H]⁺.

Step 6. To a mixture of methyl (S)-5-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-4-methoxypicolinate (1.5 g, 1.761 mmol) in MeOH (10 mL) and THF (10 mL), after degassing, was added dry palladium on carbon (0.937 g, 0.880 mmol). The reaction mixture was stirred under hydrogen atmosphere at 25° C. for 12 h and filtered through a bed of CELITE™. The filtrate was evaporated to get the crude product, methyl (S)-5-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-4-methoxypicolinate (1.1 g, 86%), which was used for the next step without further purification.

LC-MS m/z 726 [M+H]⁺.

Step 7. To a stirred solution of methyl (S)-5-((7-((1-((tert-butyldiphenylsilyl)-oxy)hexan-3-yl)amino)-5-((methoxycarbonyl)amino)-1H-pyrazolo[4,3-d]pyrimidin-1-yl)methyl)-4-methoxypicolinate (270 mg, 0.372 mmol) in THF (8 mL) and MeOH (2 mL) was added LiBH₄ (0.930 mL, 2 M solution, 1.860 mmol) at ice cold temperature. The reaction mixture was heated to 45° C. for 12 h under argon atmosphere. Additional LiBH₄ (2.5 equiv.) was added and the reaction was stirred for 5 h. The reaction was cooled to RT then crushed ice was added to get a white precipitate. The clear solution was filtered off using a cotton plug then evaporated to dryness to get methyl (S)-(7-((1-((tert-butyldiphenylsilyl)oxy)hexan-3-yl)amino)-1-((6-(hydroxymethyl)-4-methoxypyridin-3-yl)methyl)-1H-pyrazolo[4,3-d]pyrimidin-5-yl)carbamate (220 mg, 85%), which was used for the next step without further purification.

LC-MS m/z 698 [M+H]⁺.

Step 8. To a stirred solution of methyl (S)-(7-((1-((tert-butyldiphenylsilyl)oxy)hexan-3-yl)amino)-1-((6-(hydroxymethyl)-4-methoxypyridin-3-yl)methyl)-1H-pyrazolo[4,3-d]pyrimidin-5-yl)carbamate (230 mg, 0.330 mmol) in THF (5 mL) was added SOCl₂ (0.072 mL, 0.989 mmol), and the reaction mixture was stirred for 1.5 h at 0° C. The solvent was evaporated to get the crude product, methyl (S)-(7-((1-((tert-butyldiphenylsilyl)oxy)hexan-3-yl)amino)-1-((6-(chloromethyl)-4-methoxypyridin-3-yl)methyl)-1H-pyrazolo[4,3-d]pyrimidin-5-yl)carbamate (220 mg, 93%), which was used for the next step without further purification.

LC-MS m/z 716 [M+H]⁺.

Step 9. To a solution of (1S,4S)-2-methyl-2,5-diazabicyclo[2.2.1]heptane, HCl (24.90 mg, 0.168 mmol) and K₂CO₃ (57.9 mg, 0.419 mmol) in DMF (2 mL) at 0° C. was added a solution of methyl (S)-(7-((1-((tert-butyldiphenylsilyl)oxy)hexan-3-yl)amino)-1-((6-(chloromethyl)-4-methoxypyridin-3-yl)methyl)-1H-pyrazolo[4,3-d]pyrimidin-5-yl)carbamate (100 mg, 0.140 mmol) in DMF (2 mL) under inert atmosphere. The reaction mixture was stirred at 25° C. for 12 h. The reaction mixture was filtered through CELITE™ and the filtrate was evaporated to get the crude product, methyl (7-(((S)-1-((tert-butyldiphenylsilyl)oxy)hexan-3-yl)amino)-1-((4-methoxy-6-(((1S,4S)-5-methyl-2,5-diazabicyclo[2.2.1]heptan-2-yl)methyl)pyridin-3-yl)methyl)-1H-pyrazolo[4,3-d]pyrimidin-5-yl)carbamate (100 mg, 0.126 mmol, 90% yield), which was used for the next step without further purification.

LC-MS m/z 792 [M+H]⁺.

Step 10. To a solution of methyl (7-(((S)-1-((tert-butyldiphenylsilyl)oxy)hexan-3-yl)amino)-1-((4-methoxy-6-(((1S,4S)-5-methyl-2,5-diazabicyclo[2.2.1]heptan-2-yl)methyl)-pyridin-3-yl)methyl)-1H-pyrazolo[4,3-d]pyrimidin-5-yl)carbamate (100 mg, 0.126 mmol) in MeOH (5 mL) was added HCl (0.033 mL, 35 wt %, 0.379 mmol). The reaction mixture was stirred for 1 h at 25° C. The solvent was evaporated to get the crude product, methyl (7-(((S)-1-hydroxyhexan-3-yl)amino)-1-((4-methoxy-6-(((1S,4S)-5-methyl-2,5-diazabicyclo[2.2.1]heptan-2-yl)methyl)pyridin-3-yl)methyl)-1H-pyrazolo[4,3-d]pyrimidin-5-yl)carbamate (60 mg, 86%), which was used for the next step without further purification.

LC-MS m/z 554 [M+H]⁺.

Step 11. To a solution of methyl (7-(((S)-1-hydroxyhexan-3-yl)amino)-1-((4-methoxy-6-(((1S,4S)-5-methyl-2,5-diazabicyclo[2.2.1]heptan-2-yl)methyl)pyridin-3-yl)methyl)-1H-pyrazolo[4,3-d]pyrimidin-5-yl)carbamate (60 mg, 0.108 mmol) in a mixture of 1,4-dioxane (1 mL) and H₂O (1 mL) was added NaOH (4.33 mg, 0.108 mmol), and the mixture was heated at 75° C. for 12 h. The reaction mixture was cooled to RT and the layers were separated. The organic layer was evaporated and the crude material was dissolved in methanol and purified via preparative LC/MS with the following conditions: Column: Waters XBridge C18, 19×150 mm, 5-μm particles; Mobile Phase A: 10-mM NH₄OAc; Mobile Phase B: methanol; Gradient: 10-35% B over 20 minutes, then a 0-minute hold at 100% B; Flow: 20 mL/min. Fractions containing the desired product were combined and dried via centrifugal evaporation. The crude material was purified via preparative LC/MS with the following conditions: Column: Xbridge phenyl, 250 mm×19 mm, 5-μm particles; Mobile Phase A: 5:95 methanol: water with 10 m M Ammonium Bi carbonate PH-9.5 in water; Mobile Phase B: 95:5 methanol: water with 10 m M Ammonium Bi carbonate PH-9.5 in water; Gradient: a 2-minute hold at 50% B, 50-70% B over 15 minutes, then a 5-minute hold at 100% B; Flow Rate: 19 mL/min; Column Temperature: C maintained by a custom-made water bath. Fraction collection was triggered by MS and UV signals. Fractions containing the desired product were combined and dried via centrifugal evaporation to provide Compound 131 (1 mg).

The following compounds were analogously prepared: Compound 132, Compound 133, and Compound 134.

### Example 14—Compound 140

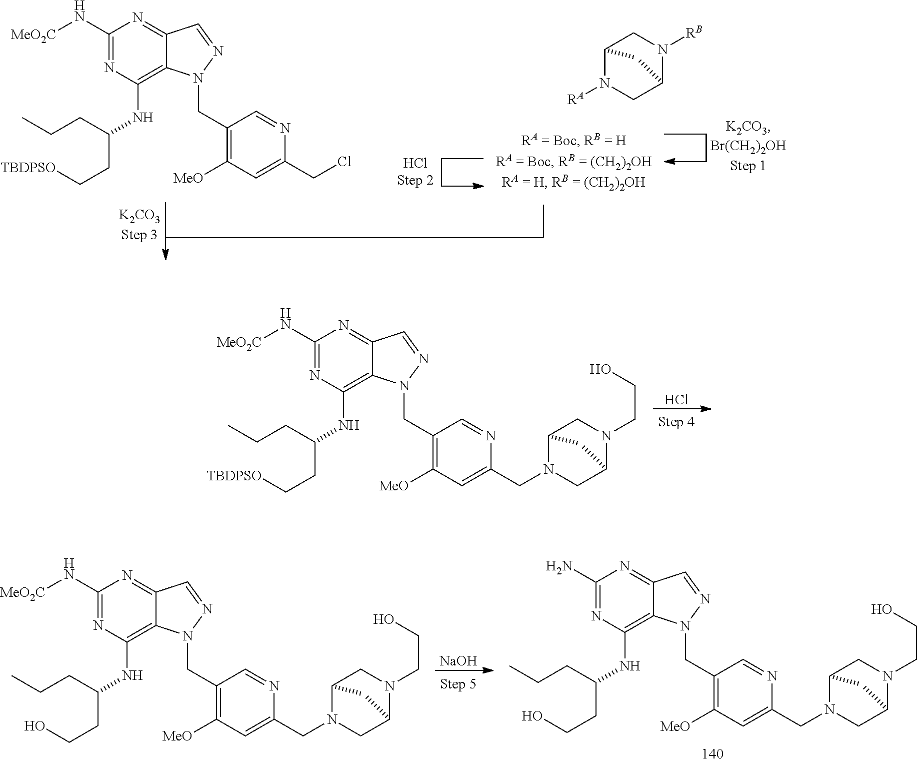

Step 1. To a solution of (1R,4R)-2,5-diaza-bicyclo[2.2.1]heptane-2-carboxylic acid tert-butyl ester (500 mg, 2.52 mmol; commercially available) in dry acetonitrile (10 mL) were added K₂CO₃ (3485 mg, 25.2 mmol) and 2-bromoethan-1-ol (630 mg, 5.04 mmol) under nitrogen atmosphere. The reaction mixture was heated at 80° C. for 12 h and partitioned between NH₄Cl solution and EtOAc. The organic layer was washed with water and brine, dried over anhydrous Na₂SO₄, and concentrated to give crude product which was purified by flash chromatography (60-120 silica gel; 1-10% MeOH in CHCl₃) to give tert-butyl (1R,4R)-5-(2-hydroxyethyl)-2,5-diazabicyclo[2.2.1]heptane-2-carboxylate (600 mg, 98%) as a light yellow oil.

LC-MS m/z 243 [M+H]⁺.

Step 2. To tert-butyl (1R,4R)-5-(2-hydroxyethyl)-2,5-diazabicyclo[2.2.1]heptane-2-carboxylate (700 mg, 2.89 mmol) in 1,4-dioxane (1 mL) was added 4 N HCl in dioxane (7.22 mL, 28.9 mmol) at 0° C. under nitrogen atmosphere. After being stirred at 0° C. for 3 h, the reaction mixture was concentrated in vacuo at 30° C. The residue was stirred with ether. The solvent was carefully decanted. The resultant solid was dried under vacuum. The solid was dissolved in a mixture of acetonitrile and water, and then it was frozen and lyophilized to afford 2-((1R,4R)-2,5-diazabicyclo[2.2.1]heptan-2-yl)ethan-1-ol, HCl (500 mg, 97%) as a white solid.

LC-MS m/z 143 [M+H]⁺.

Step 3. To a stirred mixture of 2-((1R,4R)-2,5-diazabicyclo[2.2.1]heptan-2-yl)ethan-1-ol (55.6 mg, 0.391 mmol) and K₂CO₃ (81 mg, 0.584 mmol) in DMF (2 mL) was added a solution of methyl (S)-(7-((1-((tert-butyldiphenylsilyl)oxy)hexan-3-yl)amino)-1-((6-(chloromethyl)-4-methoxypyridin-3-yl)methyl)-1H-pyrazolo[4,3-d]pyrimidin-5-yl)carbamate (140 mg, 0.195 mmol) in DMF (2 mL). The reaction mixture was stirred for 12 h at 60° C., cooled to RT, and filtered through a bed of CELITE™. The filtrate was evaporated to give crude methyl (7-(((S)-1-((tert-butyldiphenylsilyl)oxy)hexan-3-yl)amino)-1-((6-(((1R,4R)-5-(2-hydroxyethyl)-2,5-diazabicyclo[2.2.1]heptan-2-yl)methyl)-4-methoxypyridin-3-yl)methyl)-1H-pyrazolo[4,3-d]pyrimidin-5-yl)carbamate (100 mg, 62%), which was used without further purification.

Step 4. To a mixture of methyl (7-(((S)-1-((tert-butyldiphenylsilyl)oxy)hexan-3-yl)amino)-1-((6-(((1R,4R)-5-(2-hydroxyethyl)-2,5-diazabicyclo[2.2.1]heptan-2-yl)methyl)-4-methoxypyridin-3-yl)methyl)-1H-pyrazolo[4,3-d]pyrimidin-5-yl)carbamate (140 mg, 0.170 mmol) in MeOH (5 mL) was added HCl (0.026 mL, 0.851 mmol). The reaction mixture was stirred for 1.5 h at RT. The solvent was evaporated to give crude methyl (1-((6-(((1R,4R)-5-(2-hydroxyethyl)-2,5-diazabicyclo[2.2.1]heptan-2-yl)methyl)-4-methoxypyridin-3-yl)methyl)-7-(((S)-1-hydroxyhexan-3-yl)amino)-1H-pyrazolo[4,3-d]pyrimidin-5-yl)carbamate (60 mg, 60%), which was used without further purification.

Step 5. To a stirred solution of methyl (1-((6-(((1R,4R)-5-(2-hydroxyethyl)-2,5-diazabicyclo[2.2.1]heptan-2-yl)methyl)-4-methoxypyridin-3-yl)methyl)-7-(((S)-1-hydroxyhexan-3-yl)amino)-1H-pyrazolo[4,3-d]pyrimidin-5-yl)carbamate (65 mg, 0.111 mmol) in 1,4-dioxane (1 mL) was added a solution of NaOH (13.36 mg, 0.334 mmol) in H₂O (1 mL). The reaction mixture was stirred for 6 h at 80° C. The layers were separated and the organic layer was evaporated, dissolved in methanol, and purified via preparative LC/MS with the following conditions: Column: gemimi nx; Mobile Phase A: 10-mM NH₄OAc; Mobile Phase B: acetonitrile; Gradient: 10-70% B; Flow: 20 mL/min. Fractions containing the desired product were concentrated to provide Compound 140 (2.3 mg).

### Example 15—Compound 135

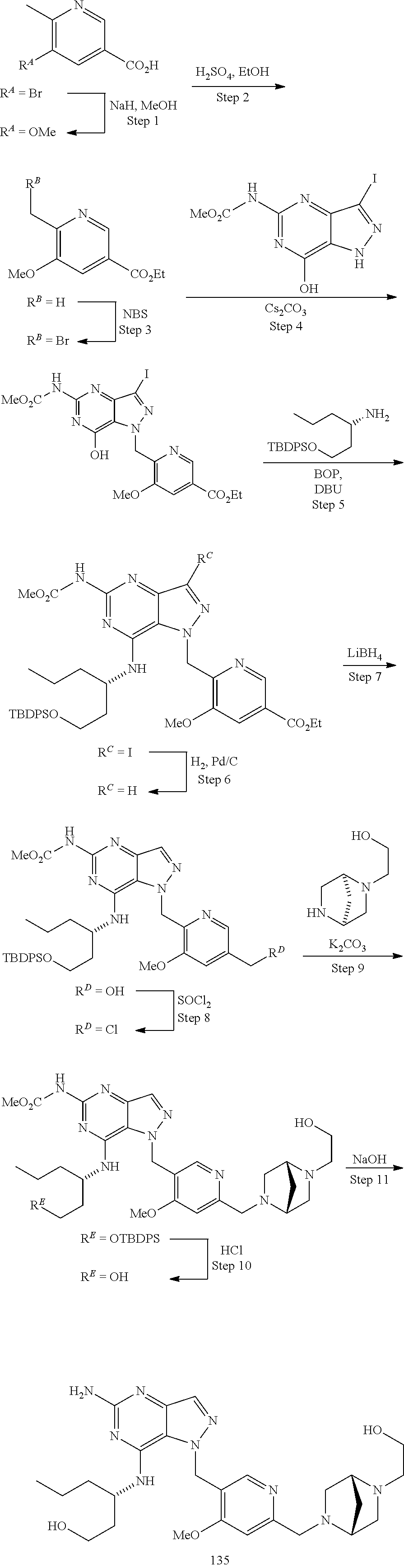

Step 1. To a stirred solution of 5-bromo-6-methylnicotinic acid (10.0 g, 46.3 mmol) in 1,4-dioxane (100.0 mL) and MeOH (18.73 mL, 463 mmol) were added Cs₂CO₃ (30.2 g, 93 mmol), Pd₂(dba)₃ (4.24 g, 4.63 mmol), and tBuXPhos (3.93 g, 9.26 mmol) under nitrogen purging. The reaction mixture was stirred at 70° C. for 16 h. The reaction mixture was filtered through a CELITE™ bed and washed with EtOAc, and the filtrate was concentrated under reduced pressure. The crude compound was treated with DCM and then filtered. The solid was washed with petroleum ether and then dried under vacuum to afford 5-methoxy-6-methylnicotinic acid (7.6 g, 98%) as a light brown solid.

LC-MS m/z 168 [M+H]⁺.

¹H NMR (300 MHz, DMSO-d₆) δ 8.42-8.32 (m, 1H), 7.64-7.55 (m, 1H), 3.79 (s, 3H), 2.32 (s, 3H).

Step 2. To a stirred solution of 5-methoxy-6-methylnicotinic acid (8.0 g, 47.9 mmol) in ethanol (80.0 mL) was added H₂SO₄ (7.65 mL, 144 mmol). The reaction mixture was stirred at 90° C. for 20 h and concentrated under reduced pressure to afford a residue, which was quenched with saturated sodium bicarbonate solution and then partitioned between DCM and water. The organic layer was washed with brine solution and dried over Na₂SO₄, filtered, and concentrated under reduced pressure to afford ethyl 5-methoxy-6-methylnicotinate (8.1 g, 80%) as a brown oil.

LC-MS m/z 196 [M+H]⁺

¹H NMR (300 MHz, DMSO-d₆) δ 8.56 (d, J=1.5 Hz, 1H), 7.66 (d, J=1.9 Hz, 1H), 4.35 (q, J=7.2 Hz, 2H), 3.90 (s, 3H), 2.43 (s, 3H), 1.34 (t, J=7.2 Hz, 3H).

Step 3. A stirred suspension of ethyl 5-methoxy-6-methylnicotinate (7.1 g, 36.4 mmol), AIBN (1.194 g, 7.27 mmol), and NBS (7.12 g, 40.0 mmol) in anhydrous CCl₄ (140 mL) was heated to 65° C. for 16 h. The reaction mixture was concentrated and the residue was purified by flash chromatography (silica gel 60-120 mesh; 15% ethyl acetate in petroleum ether as eluent) to afford ethyl 6-(bromomethyl)-5-methoxynicotinate (5.7 g, 57%) as an off-white solid.

LC-MS m/z 276 [M+H]⁺.

Step 4. To a stirred solution of methyl (7-hydroxy-3-iodo-1H-pyrazolo[4,3-d]pyrimidin-5-yl)carbamate (3.4 g, 10.15 mmol) in anhydrous DMF (50 mL) at 0° C. were added Cs₂CO₃ (6.61 g, 20.29 mmol) and ethyl 6-(bromomethyl)-5-methoxynicotinate (2.92 g, 10.65 mmol). After stirring for 1 h at 0° C., the reaction mixture was added drop-wise to ice cold water. The resulting suspension was stirred for 5 min, filtered. The collected solid was dried under high vacuum. This material was purified by flash chromatography (silica gel 60-120 mesh; 5% methanol in chloroform as eluent) to afford ethyl 6-((7-hydroxy-3-iodo-5-((methoxy-carbonyl)amino)-1H-pyrazolo[4,3-d]pyrimidin-1-yl)methyl)-5-methoxynicotinate (4.3 g, 64%) as a pale yellow solid.

LC-MS m/z 529 [M+H]⁺.

Step 5. To a stirred solution of ethyl 6-((7-hydroxy-3-iodo-5-((methoxycarbonyl)-amino)-1H-pyrazolo[4,3-d]pyrimidin-1-yl)methyl)-5-methoxynicotinate (700 mg, 1.325 mmol) in anhydrous DMSO (8 mL) were added DBU (0.599 mL, 3.98 mmol), BOP (1758 mg, 3.98 mmol) and finally (S)-1-((tert-butyldiphenylsilyl)oxy)hexan-3-amine (471 mg, 1.325 mmol) at RT. The reaction mixture was heated to 45° C., stirred for 1 h, and partitioned between water and ethyl acetate. The organic layer was washed with H₂O and saturated NaCl solution, dried over anhydrous Na₂SO₄, filtered and concentrated under reduced pressure. The residue was purified by flash chromatography (silica gel 60-120 mesh; 55% ethyl acetate in petroleum ether as eluent) to afford ethyl (S)-6-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-5-methoxynicotinate (850 mg, 52%) as a pale yellow semi-solid.

LC-MS m/z 866 [M+H]⁺.

Step 6. To a stirred solution of ethyl (S)-6-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-5-methoxynicotinate (830 mg, 0.959 mmol) in anhydrous methanol (25 mL) was added Pd/C (510 mg, 0.479 mmol) at RT. The reaction was stirred under hydrogen bladder for 16 h at RT. The suspension was filtered through CELITE™ bed and the bed was washed with ethyl acetate. The filtrate was concentrated under reduced pressure to afford ethyl (S)-6-((7-((1-((tert-butyl-diphenylsilyl)oxy)hexan-3-yl)amino)-5-((methoxycarbonyl)amino)-1H-pyrazolo[4,3-d]pyrimidin-1-yl)methyl)-5-methoxynicotinate (800 mg, 90%) as a pale yellow semi-solid.

LC-MS m/z 740 [M+H]⁺.

Step 7. To a stirred solution of ethyl (S)-6-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-5-methoxynicotinate (800 mg, 1.081 mmol) in THF (20 mL) and methanol (3.0 mL) at 0° C. was added drop-wise LiBH₄ (2.70 mL, 2 M solution, 5.41 mmol). The ice bath was removed and the reaction mixture was heated to 40° C. and stirred for 16 h. The reaction mixture was brought to RT, and additional LiBH₄ (2 mL) was added. The reaction mixture was heated to 45° C., stirred for 3 h, and cooled to 0° C. Ice cold water and ethyl acetate were added dropwise. The organic layer was washed with H₂O and saturated NaCl solution, dried over anhydrous Na₂SO₄, filtered and concentrated under reduced pressure to get methyl (S)-(7-((1-((tert-butyldiphenylsilyl)oxy)-hexan-3-yl)amino)-1-((5-(hydroxymethyl)-3-methoxypyridin-2-yl)methyl)-1H-pyrazolo[4,3-d]pyrimidin-5-yl)carbamate (750 mg, 99%) as a brown solid.

LC-MS m/z 698 [M+H]⁺.

Step 8. To a stirred solution of methyl (S)-(7-((1-((tert-butyldiphenylsilyl)oxy)hexan-3-yl)amino)-1-((5-(hydroxymethyl)-3-methoxypyridin-2-yl)methyl)-1H-pyrazolo[4,3-d]pyrimidin-5-yl)carbamate (90 mg, 0.129 mmol) in anhydrous THF (4 mL) at 0° C. was added SOCl₂ (0.047 mL, 0.645 mmol). The reaction mixture was stirred for 30 min at 0° C. and concentrated to dryness under high vacuum to get methyl (S)-(7-((1-((tert-butyldiphenylsilyl)oxy)hexan-3-yl)amino)-1-((5-(chloromethyl)-3-methoxypyridin-2-yl)methyl)-1H-pyrazolo[4,3-d]pyrimidin-5-yl)carbamate (95 mg, 93%) as a yellow solid. This material was used without further purification.

LC-MS m/z 716 [M+H]⁺.

Step 9. To a stirred solution of methyl (S)-(7-((1-((tert-butyldiphenylsilyl)oxy)hexan-3-yl)amino)-1-((5-(chloromethyl)-3-methoxypyridin-2-yl)methyl)-1H-pyrazolo[4,3-d]pyrimidin-5-yl)carbamate (90 mg, 0.126 mmol) in anhydrous DMF (2 mL) were added K₂CO₃ (52.1 mg, 0.377 mmol) and 2-((1S,4S)-2,5-diazabicyclo[2.2.1]heptan-2-yl)ethan-1-ol (35.7 mg, 0.251 mmol). The reaction mixture was heated to 75° C., stirred for 16 h, and concentrated to dryness under high vacuum to give crude methyl (7-(((S)-1-((tert-butyldiphenylsilyl)oxy)hexan-3-yl)amino)-1-((5-(((1S,4S)-5-(2-hydroxyethyl)-2,5-diazabicyclo[2.2.1]heptan-2-yl)methyl)-3-methoxypyridin-2-yl)methyl)-1H-pyrazolo[4,3-d]pyrimidin-5-yl)carbamate (120 mg), which was used without further purification.

LC-MS m/z 822 [M+H]⁺.

Step 10. To a stirred solution of methyl (7-(((S)-1-((tert-butyldiphenylsilyl)oxy)-hexan-3-yl)amino)-1-((5-(((1S,4S)-5-(2-hydroxyethyl)-2,5-diazabicyclo[2.2.1]heptan-2-yl)methyl)-3-methoxypyridin-2-yl)methyl)-1H-pyrazolo[4,3-d]pyrimidin-5-yl)carbamate (110 mg, 0.134 mmol) in anhydrous MeOH (2 mL) was added HCl (0.5 mL, 16.46 mmol) at RT. The reaction mixture was stirred for 2 h and concentrated to dryness under high vacuum to give crude methyl (1-((5-(((1S,4S)-5-(2-hydroxyethyl)-2,5-diazabicyclo[2.2.1]heptan-2-yl)methyl)-3-methoxypyridin-2-yl)methyl)-7-(((S)-1-hydroxyhexan-3-yl)amino)-1H-pyrazolo[4,3-d]pyrimidin-5-yl)carbamate (90 mg), which was used without further purification.

LC-MS m/z 584 [M+H]⁺.

Step 11. To a stirred solution of methyl (1-((5-(((1S,4S)-5-(2-hydroxyethyl)-2,5-diazabicyclo[2.2.1]heptan-2-yl)methyl)-3-methoxypyridin-2-yl)methyl)-7-(((S)-1-hydroxyhexan-3-yl)amino)-1H-pyrazolo[4,3-d]pyrimidin-5-yl)carbamate (90 mg, 0.154 mmol) in a mixture of dioxane (2 mL) and water (1 mL) was added NaOH (61.7 mg, 1.542 mmol). The reaction mixture was heated to 75° C. and stirred for 3 h. The dioxane layer from the reaction mixture was separated and concentrated to dryness to give a crude product, which was was purified via preparative LC/MS with the following conditions: Column: Waters XBridge C18, 19×150 mm, 5-μm particles; Mobile Phase A: 10-mM NH₄OAc; Mobile Phase B: acetonitrile; Gradient: 7-22% B over 20 minutes, then a 5-minute hold at 100% B; Flow: 20 mL/min. Fractions containing the desired product were combined and dried via centrifugal evaporation to provide Compound 135 (12.3 mg).

Compound 138 was analogously prepared.

### Example 16—Compound 141

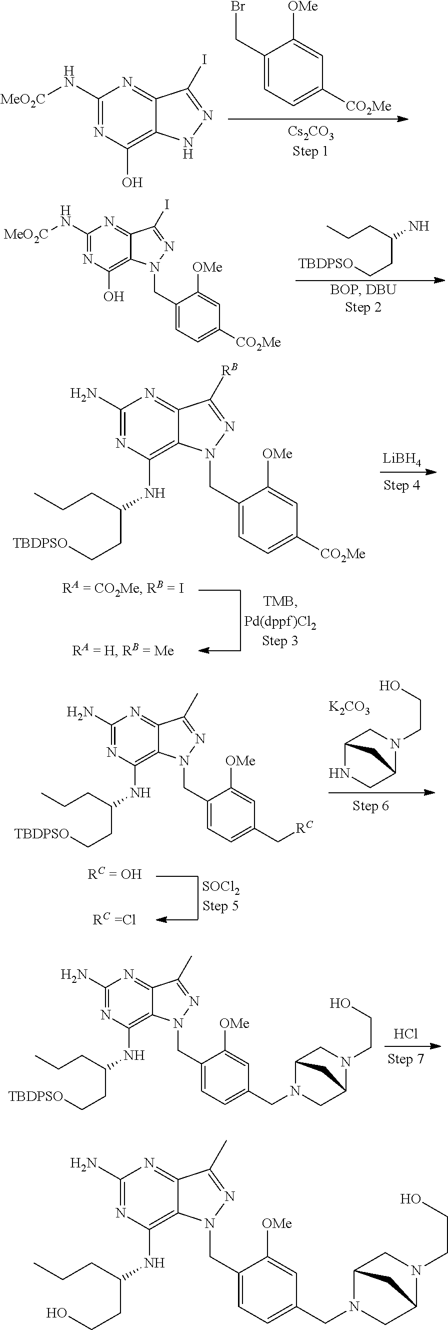

Step 1. To a stirred solution of methyl (7-hydroxy-3-iodo-1H-pyrazolo[4,3-d]pyrimidin-5-yl)carbamate (5.00 g, 14.92 mmol) in DMF (50 mL) were added Cs₂CO₃ (9.72 g, 29.8 mmol) and methyl 4-(bromomethyl)-3-methoxybenzoate (3.87 g, 14.92 mmol; commercially available). The reaction mixture was stirred at 0° C. for 1 h and partitioned between water and ethyl acetate. The organic layer was washed with brine solution, dried over anhydrous Na₂SO₄, filtered, and concentrated under vacuum to give crude product which was purified using flash chromatography (silica gel 60-120 mesh; 10% ethyl acetate in chloroform as eluent). The fraction was concentrated using high vacuum at 50° C. to give methyl 4-((7-hydroxy-3-iodo-5-((methoxycarbonyl)amino)-1H-pyrazolo[4,3-d]pyrimidin-1-yl)methyl)-3-methoxybenzoate (3.3 g, 41%) as an off-white solid.

LC-MS m/z 514 [M+H]⁺.

Step 2. To a stirred solution of methyl 4-((7-hydroxy-3-iodo-5-((methoxy-carbonyl)amino)-1H-pyrazolo[4,3-d]pyrimidin-1-yl)methyl)-3-methoxybenzoate (5 g, 9.74 mmol) in dry DMSO (10 mL) at RT were added DBU (4.41 mL, 29.2 mmol), BOP (6.46 g, 14.61 mmol), and (S)-1-((tert-butyldiphenylsilyl)oxy)hexan-3-amine (3.46 g, 9.74 mmol) in DMSO under a nitrogen atmosphere. The reaction mixture was stirred at 45° C. for 2 h and partitioned between ethyl acetate and ice cold water. The organic layer was washed with water and brine, dried over anhydrous Na₂SO₄ and concentrated in vacuo at 45° C. The crude product was purified by flash chromatography (60-120 silica gel; 20-60% ethyl acetate in petroleum ether as eluent) to afford methyl (S)-4-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-3-methoxybenzoate (4.5 g, 54%) as a light yellow oil.

LC-MS m/z 851 [M+H]⁺.

Step 3. A solution of methyl (S)-4-((7-((1-((tert-butyldiphenylsilyl)oxy)hexan-3-yl)amino)-3-iodo-5-((methoxycarbonyl)amino)-1H-pyrazolo[4,3-d]pyrimidin-1-yl)methyl)-3-methoxybenzoate (0.5 g, 0.588 mmol) in dry 1,4-dioxane (5 mL) was purged with argon for 3 min, then trimethylboroxine (TMB, 0.246 mL, 1.763 mmol), K₂CO₃ (0.162 g, 1.175 mmol), and PdCl₂(dppf)-CH₂Cl₂ adduct (0.038 g, 0.047 mmol) were added under nitrogen atmosphere. The reaction mixture was heated at 100° C. for 12 h and partitioned between sodium bicarbonate solution and DCM. The organic layer was washed with water and brine, dried over anhydrous Na₂SO₄, and concentrated get the crude product which was purified by flash chromatography (60-120 silica gel; 10-50% ethyl acetate in petroleum ether) to get methyl (S)-4-((5-amino-7-((1-((tert-butyldiphenylsilyl)oxy)hexan-3-yl)amino)-3-methyl-1H-pyrazolo[4,3-d]pyrimidin-1-yl)methyl)-3-methoxybenzoate (0.2 g, 50%) as a light yellow oil.

LC-MS m/z 681 [M+H]⁺.

Step 4. To a solution of methyl (S)-4-((5-amino-7-((1-((tert-butyldiphenylsilyl)oxy)-hexan-3-yl)amino)-3-methyl-1H-pyrazolo[4,3-d]pyrimidin-1-yl)methyl)-3-methoxybenzoate (0.2 g, 0.294 mmol) in dry tetrahydrofuran (3 mL) and MeOH (1 mL) was added LiBH₄ (0.734 mL, 2 M solution, 1.469 mmol) under nitrogen atmosphere. The reaction mixture was heated at 45° C. for 12 h and partitioned between ammonium chloride solution and EtOAc. The organic layer was washed with water and brine, dried over anhydrous Na₂SO₄ and concentrated to get the crude product which was purified by flash chromatography (60-120 silica gel; 5-55% ethyl acetate in petroleum ether as eluent) to give (S)-(4-((5-amino-7-((1-((tert-butyldiphenylsilyl)-oxy)hexan-3-yl)amino)-3-methyl-1H-pyrazolo[4,3-d]pyrimidin-1-yl)methyl)-3-methoxy-phenyl)methanol (0.3 g) as a light yellow solid.

LC-MS m/z 653 [M+H]⁺.

Step 5. To a stirred solution of (S)-(4-((5-amino-7-((1-((tert-butyldiphenylsilyl)oxy)-hexan-3-yl)amino)-3-methyl-1H-pyrazolo[4,3-d]pyrimidin-1-yl)methyl)-3-methoxyphenyl)-methanol (50 mg, 0.077 mmol) in THF (0.5 mL) at 0° C. under N₂ atmosphere was added SOCl₂ (0.011 mL, 0.153 mmol). The reaction mixture was stirred at 0° C. for 1 h under N₂ atmosphere and concentrated in vacuo to give crude (S)-N7-(1-((tert-butyldiphenylsilyl)oxy)hexan-3-yl)-1-(4-(chloromethyl)-2-methoxybenzyl)-3-methyl-1H-pyrazolo[4,3-d]pyrimidine-5,7-diamine, as a light yellowish oil, which was used without further purification.

LC-MS m/z 671 [M+H]⁺.

Step 6. To a stirred solution of (S)-N7-(1-((tert-butyldiphenylsilyl)oxy)hexan-3-yl)-1-(4-(chloromethyl)-2-methoxybenzyl)-3-methyl-1H-pyrazolo[4,3-d]pyrimidine-5,7-diamine (100 mg, 0.149 mmol) in DMF (1 mL) were added 2-((1R,4R)-2,5-diazabicyclo[2.2.1]heptan-2-yl)ethan-1-ol, HCl (53.2 mg, 0.298 mmol), and K₂CO₃ (61.8 mg, 0.447 mmol). The reaction mixture was stirred at 50° C. for 12 h and filtered to remove solids. The filtrate was concentrated in vacuo to give crude 2-((1R,4R)-5-(4-((5-amino-7-(((S)-1-((tert-butyldiphenylsilyl)oxy)-hexan-3-yl)amino)-3-methyl-1H-pyrazolo[4,3-d]pyrimidin-1-yl)methyl)-3-methoxybenzyl)-2,5-diazabicyclo[2.2.1]heptan-2-yl)ethan-1-ol, as an off white solid, which was used without further purification.

LC-MS m/z 777 [M+H]⁺.

Step 7. To a stirred solution of 2-((1R,4R)-5-(4-((5-amino-7-(((S)-1-((tert-butyldiphenylsilyl)oxy)hexan-3-yl)amino)-3-methyl-1H-pyrazolo[4,3-d]pyrimidin-1-yl)methyl)-3-methoxybenzyl)-2,5-diazabicyclo[2.2.1]heptan-2-yl)ethan-1-ol (100 mg, 0.129 mmol) in MeOH (3 mL) was added HCl (0.3 mL, 9.87 mmol). The reaction mixture was stirred at 0° C. to RT for 2 h under N₂ atmosphere and concentrated in vacuo. The residue was dissolved in MeOH in water. The solution was purified via preparative LC/MS with the following conditions: Column: Waters XBridge C18, 150 mm×19 mm, 5-μm particles; Mobile Phase A: 5:95 acetonitrile: water with 10-mM NH₄OAc; Mobile Phase B: 95:5 acetonitrile: water with 10-mM NH₄OAc; Gradient: a 0-minute hold at 5% B, 5-25% B over 20 minutes, then a 5-minute hold at 100% B; Flow Rate: 20 mL/min; Column Temperature: 25° C. Fraction collection was triggered by signals. Fractions containing the desired product were combined and dried via centrifugal evaporation to provide Compound 141 (10.6 mg).

### Example 17—Compound 137

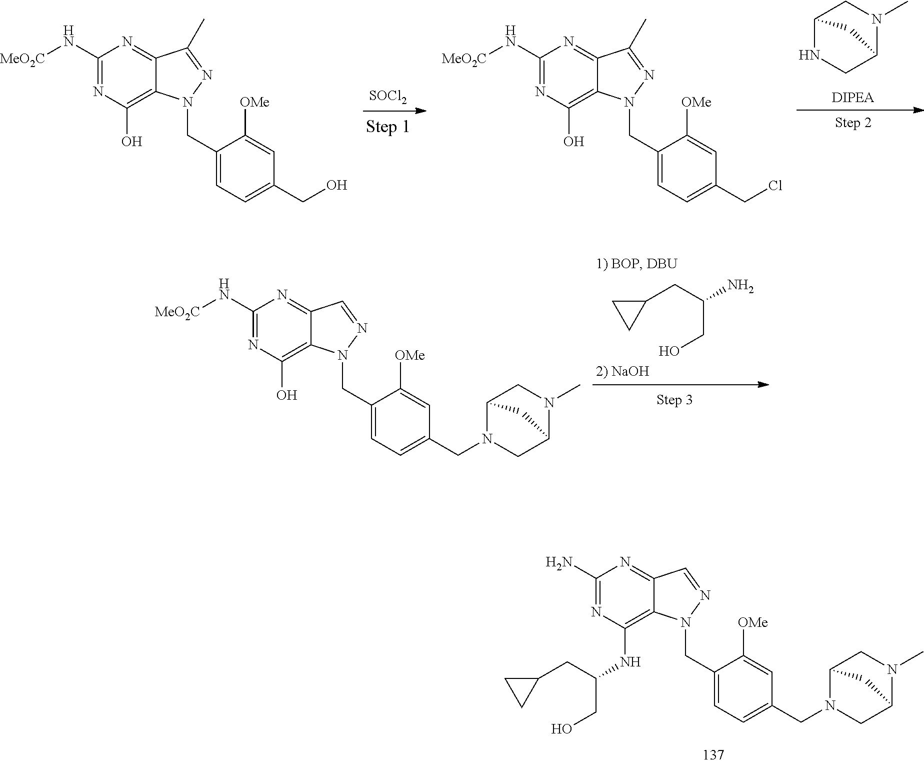

Step 1. Methyl (7-hydroxy-1-(4-(hydroxymethyl)-2-methoxybenzyl)-1H-pyrazolo[4,3-d]pyrimidin-5-yl)carbamate (304 mg, 0.846 mmol) was suspended in CH₂Cl₂ (4230 μL). SOCl₂ (309 μL, 4.23 mmol) was added and the reaction was stirred at RT for 3 h and concentrated to afford methyl (1-(4-(chloromethyl)-2-methoxybenzyl)-7-hydroxy-1H-pyrazolo[4,3-d]pyrimidin-5-yl)carbamate (311 mg).

¹H NMR (400 MHz, DMSO-d₆) δ 11.38-10.97 (m, 1H), 7.88 (s, 1H), 7.11 (d, J=1.3 Hz, 1H), 6.92 (dd, J=7.7, 1.3 Hz, 1H), 6.63 (d, J=7.7 Hz, 1H), 5.69 (s, 2H), 4.72 (s, 2H), 3.83 (s, 3H), 3.76 (s, 3H).

LC RT: 0.80 min. LC/MS [M+H]⁺=378.1 (Method 1)

Step 2. Methyl (1-(4-(chloromethyl)-2-methoxybenzyl)-7-hydroxy-1H-pyrazolo[4,3-d]pyrimidin-5-yl)carbamate (50 mg, 0.132 mmol) and (1R,4R)-2-methyl-2,5-diazabicyclo[2.2.1]-heptane, 2 hydrobromide (39.9 mg, 0.146 mmol) were suspended in acetonitrile (660 μL) and treated with DIPEA (46.2 μl, 0.265 mmol). The reaction mixture was heated to 70° C. for 16 h, cooled, and concentrated under a stream of N2. The crude material was dissolved in MeOH-DMSO and purified via preparative chromatography using the following conditions: Column: Phen Axia Luna C18, 21.2 mm×100 mm, 5-μm particles; Mobile Phase A: 90% H₂O/10% MeOH/0.1% TFA; Mobile Phase B: 10% H₂O/90% MeOH/0.1% TFA; Gradient:0-100% B over 10 minutes, then a 2-minute hold at 100% B; Flow Rate: 25 mL/min; Column Temperature: 25° C. UV Detection: 220 nm. Fractions containing the expected product were concentrated and then azeotroped from acetonitrile (2×) to afford methyl (7-hydroxy-1-(2-methoxy-4-(((1R,4R)-5-methyl-2,5-diazabicyclo[2.2.1]heptan-2-yl)methyl)benzyl)-1H-pyrazolo[4,3-d]pyrimidin-5-yl)carbamate, 2 TFA (22.3 mg).

¹H NMR (400 MHz, METHANOL-d4) δ 7.77 (s, 1H), 7.21 (br s, 1H), 7.01 (br d, J=7.8 Hz, 1H), 6.84 (d, J=7.7 Hz, 1H), 5.81 (s, 2H), 4.49-4.42 (m, 1H), 4.42-4.33 (m, 2H), 4.32-4.22 (m, 1H), 4.03-3.93 (m, 1H), 3.91 (s, 3H), 3.87 (s, 3H), 3.55-3.40 (m, 2H), 3.01 (s, 3H), 2.66-2.56 (m, 1H), 2.45 (br d, J=3.6 Hz, 1H).

LC RT: 0.58 min. LC/MS [M+H]+=454.2 (Method 1).

Step 3. Methyl (7-hydroxy-1-(2-methoxy-4-(((1R,4R)-5-methyl-2,5-diazabi-cyclo[2.2.1]heptan-2-yl)methyl)benzyl)-1H-pyrazolo[4,3-d]pyrimidin-5-yl)carbamate (19.5 mg, 0.043 mmol), (S)-2-amino-3-cyclopropylpropan-1-ol, HCl (13.04 mg, 0.086 mmol) and BOP, 98%, 25 g (33.6 mg, 0.064 mmol) were suspended in dioxane (430 μl) at RT. The reaction mixture was treated with DBU (25.9 μl, 0.172 mmol) and stirred at RT for 72 h. Another portion of 7 mg of (S)-2-amino-3-cyclopropylpropan-1-ol, HCl (13.04 mg, 0.086 mmol) and 13 μL of DBU were added and the reaction was heated to 40° C. for ˜4 h. 10 M aqueous NaOH solution (43.0 μl, 0.430 mmol) was added. The temperature was increased to 60° C. and the reaction mixture was stirred overnight. The mixture containing the crude product was concentrated, diluted with DMF/1N HCl solution (430 μL). The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 0% B, 0-40% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide Compound 137 (1.7 mg) as the free base.

### Example 18—Compound 139

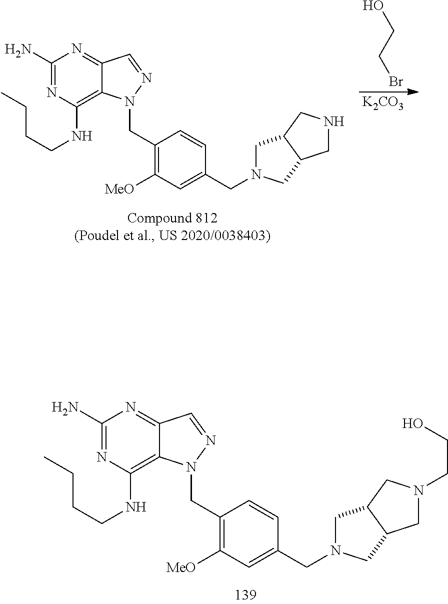

A solution of Compound 812 (US 2020/0038403; 18 mg, 0.040 mmol) in DMF (0.5 mL) was treated with K₂CO₃ (16.56 mg, 0.120 mmol) and 2-bromoethan-1-ol (5.66 μl, 0.080 mmol). The reaction mixture and heated at 50° C. for 2 h. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 7% B, 7-47% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The material was further purified via preparative LC/MS with the following conditions: Column: XBridge Phenyl, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to provide 4.3 mg of Compound 139.

### Example 19—Compound 142

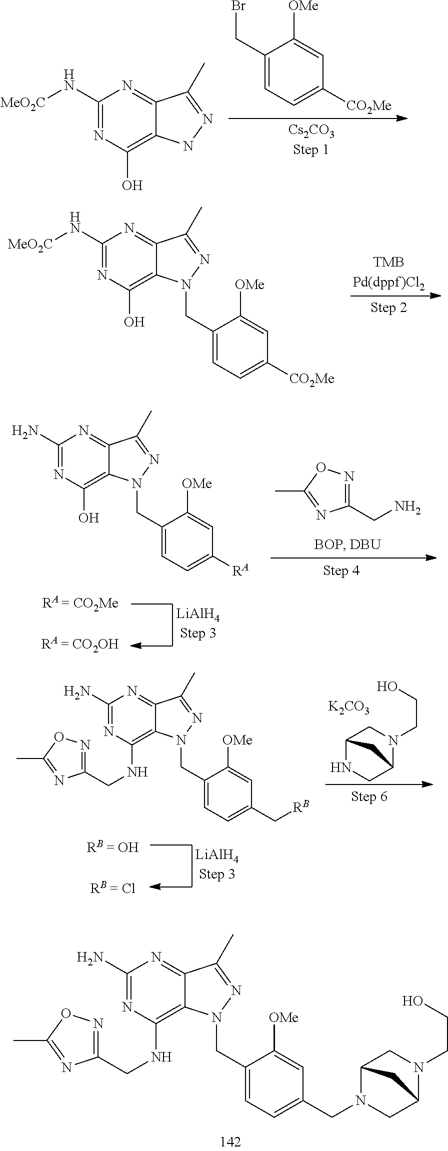

Step 1. To a stirred solution of methyl (7-hydroxy-3-iodo-1H-pyrazolo[4,3-d]pyrimidin-5-yl)carbamate (5.0 g, 14.92 mmol) in DMF (50.0 mL) at 0° C., were added Cs₂CO₃ (9.72 g, 29.8 mmol) and methyl 4-(bromomethyl)-3-methoxybenzoate (3.87 g, 14.92 mmol). The reaction mixture was stirred at 0° C. for 1 h and water was added. The precipitated solid was filtered, washed with excess of water followed by petroleum ether and dried under vacuum. The crude compound was purified by ISCO combiflash chromatography by eluting with 0-100% ethyl acetate in chloroform to afford methyl 4-((7-hydroxy-3-iodo-5-((methoxy-carbonyl)amino)-1H-pyrazolo[4,3-d]pyrimidin-1-yl)methyl)-3-methoxybenzoate (3.88 g, 6.20 mmol, 41.5% yield) as an off-white solid.

¹H NMR (400 MHz, DMSO-d₆) δ ppm: 11.69 (br s, 1H), 11.38 (s, 1H), 7.56-7.45 (m, 2H), 6.87-6.78 (m, 1H), 5.75 (s, 2H), 3.88 (s, 3H), 3.85 (s, 3H), 3.75 (s, 3H). LC-MS m/z 514.0 [M+H]⁺.

Step 2. To a stirred solution of methyl 4-((7-hydroxy-3-iodo-5-((methoxycarbonyl)-amino)-1H-pyrazolo[4,3-d]pyrimidin-1-yl)methyl)-3-methoxybenzoate (3.5 g, 6.82 mmol) in 1,4-dioxane (35.0 mL), were added K₂CO₃ (1.885 g, 13.64 mmol), trimethylboroxine (TMB, 1.907 mL, 13.64 mmol) and PdCl₂(dppf). CH₂Cl₂ adduct (0.557 g, 0.682 mmol) under nitrogen purging. The reaction mixture was stirred at 100° C. for 6 h. The reaction mixture was filtered through a CELITE™ bed, which was subsequently washed with excess of ethyl acetate. The filtrate was concentrated under reduced pressure to afford a residue. The crude compound was purified by ISCO combiflash chromatography (0-20% methanol in chloroform) to afford methyl 4-((5-amino-7-hydroxy-3-methyl-1H-pyrazolo[4,3-d]pyrimidin-1-yl)methyl)-3-methoxybenzoate (2.1 g, 4.10 mmol, 60.1% yield) as a brown solid.

¹H NMR (400 MHz, DMSO-d₆) δ=10.90 (s, 1H), 7.51 (s, 1H), 7.46 (d, J=8.0 Hz, 1H), 6.63-6.50 (m, 1H), 6.18-6.01 (m, 2H), 5.71-5.54 (m, 2H), 3.91 (s, 3H), 3.87-3.78 (s, 3H), 2.23 (s, 3H).

LC-MS m/z 344.0 [M+H]⁺.

Step 3. To a stirred solution of methyl 4-((5-amino-7-hydroxy-3-methyl-1H-pyrazolo[4,3-d]pyrimidin-1-yl)methyl)-3-methoxybenzoate (0.5 g, 1.456 mmol) in THF (5.0 mL) at 0° C., was added LiAlH₄ (1.214 mL, 2.91 mmol). The reaction mixture was warmed to RT, stirred for 1 h, quenched with ice cold water, and filtered through a CELITE™ bed, which was washed with excess of ethyl acetate. The organic layer was dried over Na₂SO₄, filtered and concentrated under reduced pressure to afford 5-amino-1-(4-(hydroxymethyl)-2-methoxy-benzyl)-3-methyl-1H-pyrazolo[4,3-d]pyrimidin-7-ol (0.31 g, 0.551 mmol, 37.8% yield) as a brown semi-solid.

¹H NMR (400 MHz, DMSO-d₆) δ=6.99-6.95 (m, 1H), 6.73 (br d, J=7.5 Hz, 1H), 6.44-6.38 (m, 1H), 5.75-5.49 (m, 2H), 5.26-4.99 (m, 1H), 4.44 (s, 2H), 3.87-3.80 (m, 3H), 2.23 (s, 3H).

LC-MS m/z 316.3 [M+H]⁺.

Step 4. To a stirred solution of 5-amino-1-(4-(hydroxymethyl)-2-methoxybenzyl)-3-methyl-1H-pyrazolo[4,3-d]pyrimidin-7-ol (1.1 g, 3.49 mmol) in DMSO (10.0 mL), were added DBU (1.577 mL, 10.47 mmol), BOP (2.314 g, 5.23 mmol) and (5-methyl-1,2,4-oxadiazol-3-yl)methanamine hydrochloride (0.522 g, 3.49 mmol). The reaction mixture was stirred at RT for 2 h. (5-Methyl-1,2,4-oxadiazol-3-yl)methanamine hydrochloride (0.3 g, 2.0 mmol) was added. The reaction mixture was stirred at RT for 16 h and partitioned between EtOAc and water. The organic layer was washed with brine, dried over Na₂SO₄, filtered and concentrated under reduced pressure to afford a residue. The crude compound was purified by ISCO combiflash chromatography by eluting with 0-20% methanol in chloroform to afford (4-((5-Amino-3-methyl-7-(((5-methyl-1,2,4-oxadiazol-3-yl)methyl)amino)-1H-pyrazolo[4,3-d]pyrimidin-1-yl)methyl)-3-methoxyphenyl)methanol (0.81 g, 1.243 mmol, 35.6% yield) as a brown solid.

¹H NMR (400 MHz, DMSO-d₆) δ=7.60-7.55 (m, 1H), 7.26 (br t, J=5.8 Hz, 1H), 6.98-6.93 (m, 1H), 6.77 (br d, J=7.5 Hz, 1H), 6.68-6.60 (m, 1H), 5.68 (s, 2H), 5.55-5.48 (m, 1H), 5.20-5.13 (m, 1H), 4.78 (br d, J=5.5 Hz, 2H), 4.49-4.42 (m, 2H), 3.82-3.77 (m, 3H), 2.56 (d, J=2.0 Hz, 4H), 2.55-2.50 (m, 6H).

LC-MS m/z 411.2 [M+H]⁺.

Step 5. To a stirred solution of (4-((5-amino-3-methyl-7-(((5-methyl-1,2,4-oxadiazol-3-yl)methyl)amino)-1H-pyrazolo[4,3-d]pyrimidin-1-yl)methyl)-3-methoxyphenyl)methanol (0.45 g, 1.096 mmol) in THF (10.0 mL) at 0° C., was added SOCl₂ (1.0 ml, 13.70 mmol). The reaction mixture was stirred at 0° C. for 1 h, warmed to RT, and concentrated under reduced pressure to afford 1-(4-(chloromethyl)-2-methoxybenzyl)-3-methyl-N7-((5-methyl-1,2,4-oxadiazol-3-yl)methyl)-1H-pyrazolo[4,3-d]pyrimidine-5,7-diamine (0.51 g, assumed 100% yield) as a brown solid. The crude product was used as such in the next step.

LC-MS m/z 429.4 [M+H]⁺.

Step 6. To a stirred solution of 1-(4-(chloromethyl)-2-methoxybenzyl)-3-methyl-N7-((5-methyl-1,2,4-oxadiazol-3-yl)methyl)-1H-pyrazolo[4,3-d]pyrimidine-5,7-diamine (0.15 g, 0.350 mmol) in DMF (3.0 mL), were added 2-((1S,4S)-2,5-diazabicyclo[2.2.1]heptan-2-yl)ethan-1-ol hydrochloride (0.094 g, 0.525 mmol) and K₂CO₃ (0.048 g, 0.350 mmol). The reaction mixture was stirred at 50° C. for 90 min. The reaction mixture was filtered through a CELITE™ bed, which was subsequently washed with excess of ethyl acetate. The filtrate was concentrated under reduced pressure to afford a residue. The crude compound was purified by reversed phase preparative LC/MS (Column: TRIART-YMC EXRS-(250×19 mm), mobile phase A:10 mM NH₄HCO₃; mobile phase B: acetonitrile:MeOH (1:1); gradient: 0/20, 2/20 10/40, 15/40 18/100 20/20, flow rate: 19 mL/min). The fraction collection was triggered by MS and UV signals. Fractions containing the desired product were combined and dried via centrifugal evaporation using a Genevac apparatus to afford Compound 142 (33.2 mg, 0.062 mmol, 17.76% yield).

### Example 20—Starting Materials and Intermediates

The Charts below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

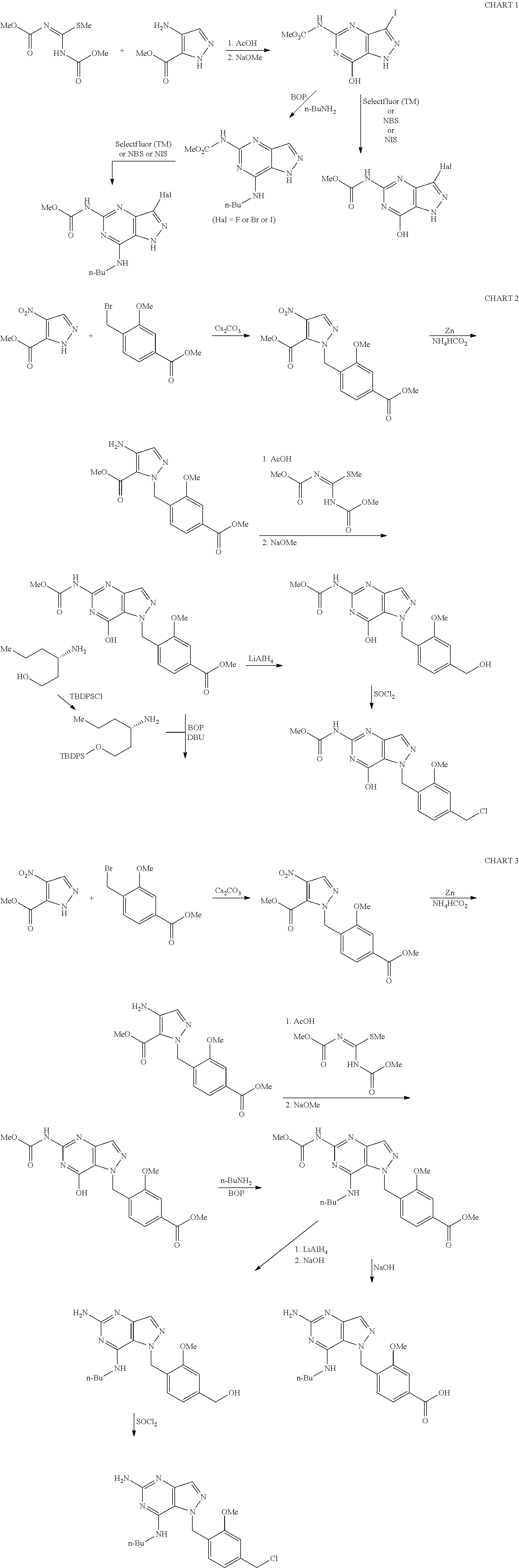

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue™ reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×→1× in H₂O, warm at 37° C.; Cat #BD 558049) and kept the perm buffer (on ice) for later use.

For surface markers staining (CD69): prepared surface Abs: 0.045 ul hCD14-FITC (ThermoFisher Cat #MHCD1401)+0.6 ul hCD19-ef450 (ThermoFisher Cat #48-0198-42)+1.5 ul hCD69-PE (cat #BD555531)+0.855 ul FACS buffer. Added 3 ul/well, spin1000 rpm for 1 min and mixed on shaker for 30 sec, put on ice for 30 mins. Stop stimulation after 30 min with 70 uL of prewarmed 1× fix/lysis buffer and use Feliex mate to resuspend (15 times, change tips for each plate) and incubate at 37C for 10 min.

Centrifuge at 2000 rpm for 5 min aspirate with HCS plate washer, mix on shaker for 30 sec and then wash with 70 uL in dPBS and pelleted 2× s (2000 rpm for 5 min) and 50 ul wash in FACS buffer pelleted 1×s(2000 rpm for 5 min). Mix on shaker for 30 sec. For Intracellular markers staining (MX-1): Add 50 ul of BD Perm buffer III and mix on shaker for 30 sec. Incubate on ice for 30 min (in the dark). Wash with 50 uL of FACS buffer 2× (spin @2300 rpm×5 min after perm) followed by mixing on shaker for 30 sec. Resuspended in 20 ul of FACS buffer containing MX1 antibody ( )(4812)-Alexa 647: Novus Biologicals #NBP2-43704AF647) 20 ul FACS bf+0.8 ul hlgG+0.04 ul MX-1. Spin 1000 rpm for 1 min, mix on shaker for 30se and the samples were incubated at RT in the dark for 45 min followed by washing 2× FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-Alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% CO₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by Thermo-Fisher Scientific). A 70 uL volume of mRNA catcher lysis buffer (1×) with DTT from the Invitrogen mRNA Catcher Plus kit (Cat #K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat #K1570-02) according to the manufacturer's instructions. Half yield of mRNA from RNA extraction were used to synthesize cDNA in 20 μL reverse transcriptase reactions using Invitrogen SuperScript IV VILO Master Mix (Cat #11756500). TaqMan® real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

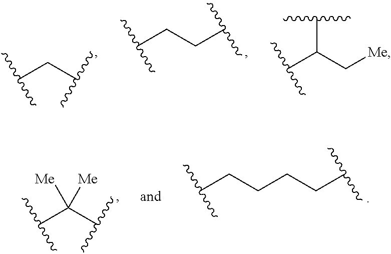

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z—) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cyclo-alkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (osr “bicycloalkylene”) and “spiroalkanediyl” (or “spiroalkylene”) refer to divalent counterparts of a bicycloalkyl and spiroalkyl (or “spirocycloalkyl”) group.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzo-furanyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond or an asterisk (*) at the end of the bond denotes a covalent attachment site. For instance, a statement that R is

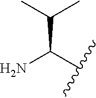 [SMILES: *[C@@H](N)C(C)C]

or that R is

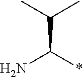 [SMILES: *[C@@H](N)C(C)C]

in the formula

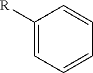

means

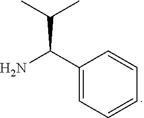

In the formulae of this specification, a bond traversing an aromatic ring between two carbons thereof means that the group attached to the bond may be located at any of the positions of the aromatic ring made available by removal of the hydrogen that is implicitly there (or explicitly there, if written out). By way of illustration:

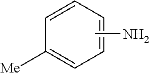

represents

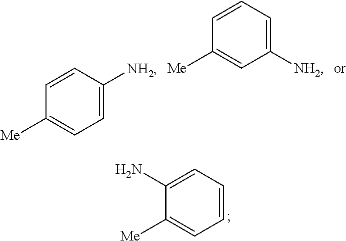 [SMILES: Cc1cccc(N)c1 ; Cc1ccc(N)cc1 ; Cc1ccccc1N]

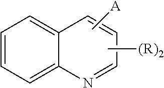

represents

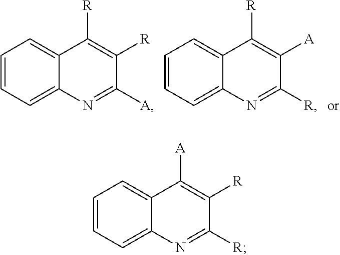

and

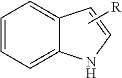

represents

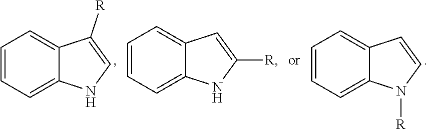

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### Acronyms and Abbreviations

Table C provides a list of acronyms and abbreviations used in this specification, along with their meanings.

TABLE C

| ACRONYM OR | |
|---|---|
| ABBREVIATION | MEANING OR DEFINITION |
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hunig's base |
| DMA | N,N-Dimethylacetamide |
| DMAP | 4-(Dimethylamino)pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenyl methyloxycarbonyl |
| HATU | Hexafluorophosphate Azabenzotriazole |
| | Tetramethyl Uronium; 1-[Bis(dimethylamino)- |
| | methylene]-1H-1,2,3-triazolo[4,5- |
| | b]pyridinium 3-oxide hexafluorophosphate |
| HEPES | 4-(2-Hydroxyethyl)piperazine-1-ethanesulfonic |
| | acid, N-(2-Hydroxyethyl)piperazine-N′-(2- |
| | ethanesulfonic acid) |
| HPLC | High pressure liquid chromatography |
| Hunig's base | See DIPEA, DIEA |
| LCMS, LC-MS, LC/MS | Liquid chromatography-mass spectrometry |
| mCPBA | m-chloroperbenzoic acid |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluoroethylene) |
| RT (in context of liquid | Retention time, in min |
| chromatography) | |
| RT (in the context of | Room (ambient) temperature, circa 25° C. |
| reaction conditions) | |
| Sat. | Saturated |
| Soln | Solution |
| TBDPS | tert-Butyldiphenylsilyl |
| TBS | t-Butyldimethylsilyl group |
| TEA | Triethylamine |
| TEAA | Triethylammonium acetate |
| TFA | Trifluoroacetic acid |
| THF | Tetrahydrofuran |

### REFERENCES

Full citations for the following references cited in abbreviated fashion by first author (or inventor) and date earlier in this specification are provided below. Each of these references is incorporated herein by reference for all purposes.
Akinbobuyi et al., Tetrahedron Lett. 2015, 56, 458, “Facile syntheses of functionalized toll-like receptor 7 agonists”.
Akinbobuyi et al., Bioorg. Med. Chem. Lett. 2016, 26, 4246, “Synthesis and immunostimulatory activity of substituted TLR7 agonists.”
Barberis et al., US 2012/0003298 A1 (2012).
Beesu et al., J. Med. Chem. 2017, 60, 2084, “Identification of High-Potency Human TLR8 and Dual TLR7/TLR8 Agonists in Pyrimidine-2,4-diamines.”
Berghöfer et al., J. Immunol. 2007, 178, 4072, “Natural and Synthetic TLR7 Ligands Inhibit CpG-A- and CpG-C-Oligodeoxynucleotide-Induced IFN-α Production.”
Bonfanti et al., US 2014/0323441 A1 (2015) [2015a].
Bonfanti et al., US 2015/0299221 A1 (2015) [2015b].
Bonfanti et al., US 2016/0304531 A1 (2016).
Carson et al., US 2013/0202629 A1 (2013).
Carson et al., U.S. Pat. No. 8,729,088 B2 (2014).
Carson et al., U.S. Pat. No. 9,050,376 B2 (2015).
Carson et al., US 2016/0199499 A1 (2016).
Chan et al., Bioconjugate Chem. 2009, 20, 1194, “Synthesis and Immunological Characterization of Toll-Like Receptor 7 Agonistic Conjugates.”
Chan et al., Bioconjugate Chem. 2011, 22, 445, “Synthesis and Characterization of PEGylated Toll Like Receptor 7 Ligands.”
Chen et al., U.S. Pat. No. 7,919,498 B2 (2011).
Coe et al., U.S. Pat. No. 9,662,336 B2 (2017).
Cortez and Va, Medicinal Chem. Rev. 2018, 53, 481, “Recent Advances in Small-Molecule TLR7 Agonists for Drug Discovery”.
Cortez et al., US 2017/0121421 A1 (2017).
Cortez et al., U.S. Pat. No. 9,944,649 B2 (2018).
Dellaria et al., WO 2007/028129 A1 (2007).
Desai et al., U.S. Pat. No. 9,127,006 B2 (2015).
Ding et al., WO 2016/107536 A1 (2016).
Ding et al., US 2017/0273983 A1 (2017) [2017a].
Ding et al., WO 2017/076346 A1 (2017) [2017b].
Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”
Graupe et al., U.S. Pat. No. 8,993,755 B2 (2015).
Embrechts et al., J. Med. Chem. 2018, 61, 6236, “2,4-Diaminoquinazolines as Dual Toll Like Receptor (TLR) 7/8 Modulators for the Treatment of Hepatitis B Virus.”
Halcomb et al., U.S. Pat. No. 9,161,934 B2 (2015).
Hashimoto et al., US 2009/0118263 A1 (2009).
He et al., U.S. Pat. No. 10,487,084 B2 (2019) [2019a].
He et al., U.S. Pat. No. 10,508,115 B2 (2019) [2019b].
Hirota et al., U.S. Pat. No. 6,028,076 (2000).
Holldack et al., US 2012/0083473 A1 (2012).
Isobe et al., U.S. Pat. No. 6,376,501 B1 (2002).
Isobe et al., JP 2004137157 (2004).
Isobe et al., J. Med. Chem. 2006, 49 (6), 2088, “Synthesis and Biological Evaluation of Novel 9-Substituted-8-Hydroxyadenine Derivatives as Potent Interferon Inducers.”
Isobe et al., U.S. Pat. No. 7,521,454 B2 (2009) [2009a].
Isobe et al., US 2009/0105212 A1 (2009) [2009b].
Isobe et al., US 2011/0028715 A1 (2011).
Isobe et al., U.S. Pat. No. 8,148,371 B2 (2012).
Jensen et al., WO 2015/036044 A1 (2015).
Jones et al., U.S. Pat. No. 7,691,877 B2 (2010).
Jones et al., US 2012/0302598 A1 (2012).
Kasibhatla et al., U.S. Pat. No. 7,241,890 B2 (2007).
Koga-Yamakawa et al., Int. J. Cancer 2013, 132 (3), 580, “Intratracheal and oral administration of SM-276001: A selective TLR7 agonist, leads to antitumor efficacy in primary and metastatic models of cancer.”
Li et al., U.S. Pat. No. 9,902,730 B2 (2018).
Lioux et al., U.S. Pat. No. 9,295,732 B2 (2016).
Lund et al., Proc. Nat'l Acad. Sci (USA) 2004, 101 (15), 5598, “Recognition of single-stranded RNA viruses by Toll-like receptor 7.”
Maj et al., U.S. Pat. No. 9,173,935 B2 (2015).
McGowan et al., US 2016/0168150 A1 (2016) [2016a].
McGowan et al., U.S. Pat. No. 9,499,549 B2 (2016) [2016b].
McGowan et al., J. Med. Chem. 2017, 60, 6137, “Identification and Optimization of Pyrrolo[3,2-d]pyrimidine Toll-like Receptor 7 (TLR7) Selective Agonists for the Treatment of Hepatitis B.”
Musmuca et al., J. Chem. Information & Modeling 2009, 49 (7), 1777, “Small-Molecule Interferon Inducers. Toward the Comprehension of the Molecular Determinants through Ligand-Based Approaches.”
Nakamura et al., Bioorg. Med. Chem. Lett. 2013, 13, 669, “Synthesis and evaluation of 8-oxoadenine derivatives as potent Toll-like receptor agonists with high water solubility.”
Ogita et al., US 2007/0225303 A1 (2007).
Ota et al., WO 2019/124500 A1 (2019).
Pilatte et al., WO 2017/216293 A1 (2017).
Poudel et al., U.S. Pat. No. 10,472,361 B2 (2019) [2019a].
Poudel et al., U.S. Pat. No. 10,494,370 B2 (2019) [2019b].
Poudel et al., US 2020/0083403 A1 (2020) [2020a].
Poudel et al., US 2020/0039986 A1 (2020) [2020b].
Purandare et al., WO 2019/209811 A1 (2019).
Pryde, U.S. Pat. No. 7,642,350 B2 (2010).
Sato-Kaneko et al., JCI Insight 2017, 2, e93397, “Combination Immunotherapy with TLR Agonists and Checkpoint Inhibitors Suppresses Head and Neck Cancer”.
Smits et al., The Oncologist 2008, 13, 859, “The Use of TLR7 and TLR8 Ligands for the Enhancement of Cancer Immunotherapy”.
Vasilakos and Tomai, Expert Rev. Vaccines 2013, 12, 809, “The Use of Toll-like Receptor 7/8 Agonists as Vaccine Adjuvants”.
Vernejoul et al., US 2014/0141033 A1 (2014).
Young et al., U.S. Pat. No. 10,457,681 B2 (2019).
Yu et al., PLoS One 2013, 8 (3), e56514, “Toll-Like Receptor 7 Agonists: Chemical Feature Based Pharmacophore Identification and Molecular Docking Studies.”
Zhang et al., Immunity 2016, 45, 737, “Structural Analysis Reveals that Toll-like Receptor 7 Is a Dual Receptor for Guanosine and Single-Stranded RNA.”
Zhang et al., WO 2018/095426 A1 (2018)>
Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula I 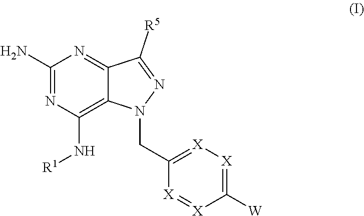
wherein
W is 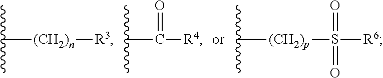
each X is independently N or CR²;
R¹ is (C₁-C₅ alkyl),
(C₂-C₅ alkenyl),
(C₁-C₈ alkanediyl)₀₋₁(C₃-C₆ cycloalkyl),
(C₂-C₈ alkanediyl)OH,
(C₂-C₈ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
(C2-C₈ alkanediyl)₀₋₁(C₃-C₆ cycloalkanediyl)(C₃-C₆ cycloalkyl),
or
(C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
R³ is NH[C(═O)]₀₋₁(C₁-C₄ alkanediyl)₀₋₁(C₄-C₁₀ bicycloalkyl), O(C₁-C₄ alkanediyl)₀₋₁(C₄-C₈ bicycloalkyl),
or
a moiety having the structure 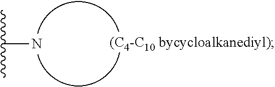 [SMILES: *N]
R⁴ is NH(C₁-C₄ alkanediyl)₀₋₁(C₄-C₁₀ bicycloalkyl)
or
a moiety having the structure 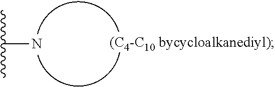 [SMILES: *N]
R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl, 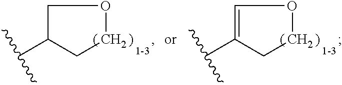 [SMILES: *C1CCOC1 ; *C1=COCC1]
R⁶ is (NH)₀₋₁(C₁-C₄ alkanediyl)₀₋₁(C4-C₁₀ bicycloalkyl),
or
a moiety having the structure 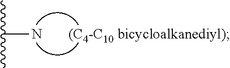 [SMILES: *N]
R^{x} and R^{y} are independently H or C₁-C₃ alkyl or R^{x} and R^{y} combine with the nitrogen to which they are bonded to form a 3- to 7-membered heterocycle;
n is 1, 2, or 3;
and
p is 0, 1, 2, or 3;
wherein in R¹, R², R³, R⁴, R⁵, and R⁶
an alkyl, alkenyl, cycloalkyl, alkanediyl, bicycloalkyl, or a moiety of the formula 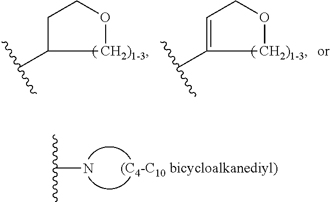
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkenyl, alkanediyl, cycloalkyl, bicycloalkyl, or a moiety of the formula 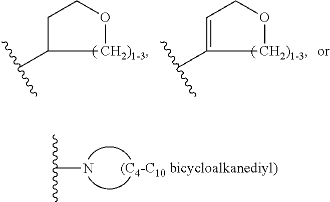
optionally may have a CH₂ group replaced by O, SO₂, CF₂, C(═O), NH,
N[C(═O)]₀₋₁(C₁-C₅ alkyl),
N[C(═O)]₀₋₁(C₁-C₄ alkanediyl)CF₃,
N[C(═O)]₀₋₁(C₂-C₄ alkanediyl)OH
N(SO₂)(C₁-C₃ alkyl),
N(C₁-C₃ alkanediyl)₀₋₁[C(═O)]N(C₁-C₃ alkyl)₂,
or
N[C(═O)]₀₋₁(C₁-C₄ alkanediyl)₀₋₁(C₃-C₅ cycloalkyl);
with the proviso that the compound of formula (I) is not

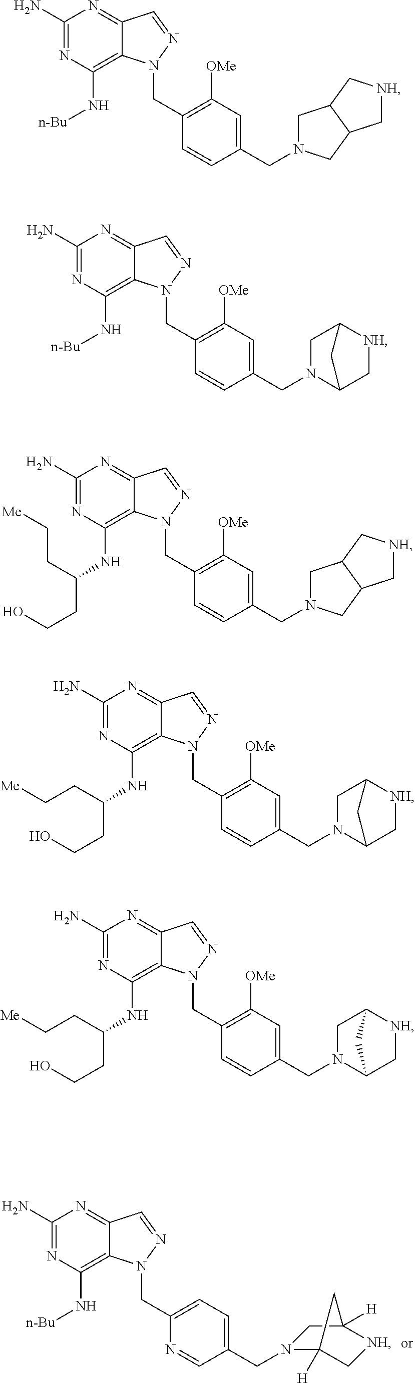

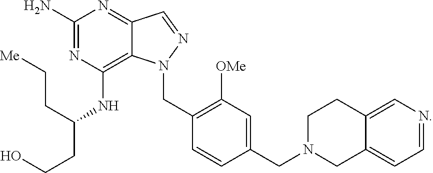 [SMILES: CCC[C@@H](CCO)Nc1nc(N)nc2cnn(Cc3ccc(CN4CCc5cnccc5C4)cc3OC)c12]

2. A compound according to claim 1, wherein W is

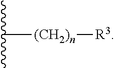

3. A compound according to claim 2, wherein W is selected from the group consisting of

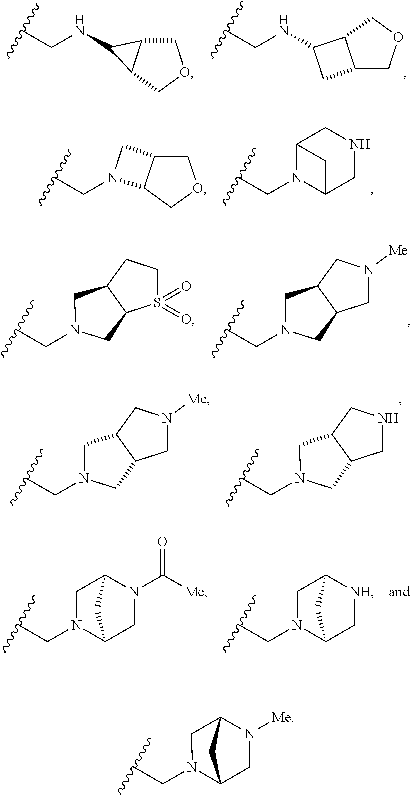

4. A compound according to claim 4, wherein W is

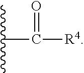

5. A compound according to claim 4, wherein W is selected from the group consisting of

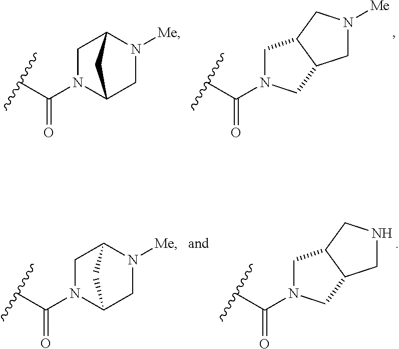

6. A compound according to claim 1, wherein R¹ is selected from the group consisting of

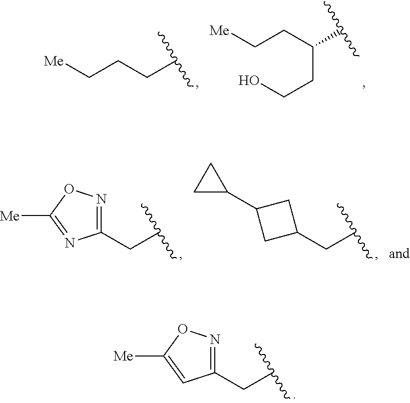 [SMILES: *CCCC ; *[C@@H](CCC)CCO ; *Cc1noc(C)n1 ; *CC1CC(C2CC2)C1 ; *Cc1cc(C)on1]

7. A compound according to claim 1, wherein R² is OMe, O(cyclopropyl), or OCHF₂.

8. A compound according to claim 1, wherein R⁵ is H, CH₂OH, or Me.

9. A compound according to claim 1, having a structure according to formula (Ia)

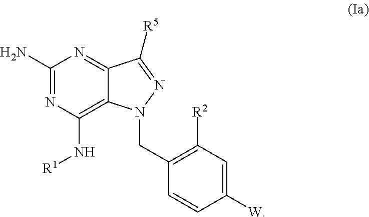

10. A compound according to claim 1, having a structure according to formula (Ib)

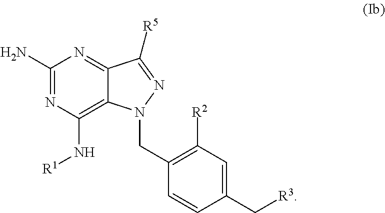

11. A compound according to claim 1, having a structure according to formula (Ic)

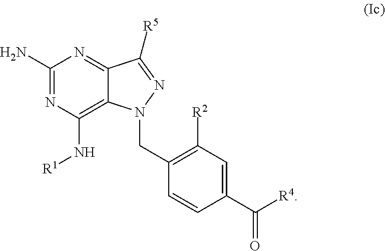

12. A compound having a structure according to formula (Ia) 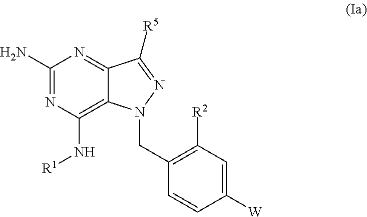
wherein
R¹ is 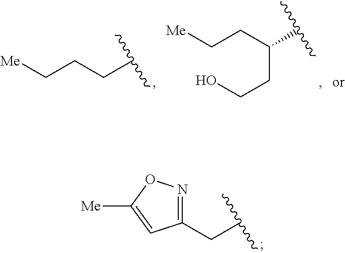 [SMILES: *CCCC ; *[C@@H](CCC)CCO ; *Cc1cc(C)on1]
R² is OMe or OCHF₂;
R⁵ is H or Me; and
W is

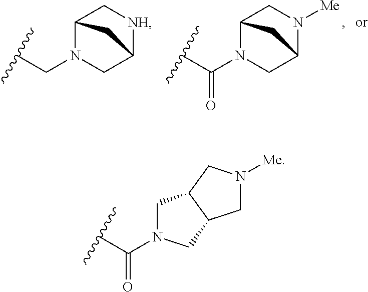 [SMILES: *CN1C[C@@H]2C[C@H]1CN2 ; *C(=O)N1C[C@@H]2C[C@H]1CN2C ; *C(=O)N1C[C@H]2CN(C)C[C@H]2C1]

13. A method of treating a cancer, comprising administering to a patient suffering from such cancer a therapeutically effective combination of an anti-cancer immunotherapy agent and a compound according to claim 1.

14. A method according to claim 13, wherein the anti-cancer immunotherapy agent is an antagonistic anti-CTLA-4, anti-PD-1, or anti-PD-L1 antibody.

15. A method according to claim 13, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

16. A method according to claim 15, wherein the anti-cancer immunotherapy agent is ipilimumab, nivolumab, or pembrolizumab.

17. A compound according to claim 1, having a structure represented by formula (Id):

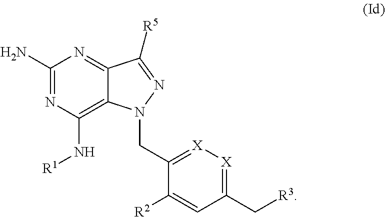

18. A method of treating a cancer, comprising administering to a patient suffering from such cancer a therapeutically effective combination of an anti-cancer immunotherapy agent and a compound according to claim 12.

19. A method according to claim 18, wherein the anti-cancer immunotherapy agent is an antagonistic anti-CTLA-4, anti-PD-1, or anti-PD-L1 antibody.

20. A method according to claim 18, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.
